SEQUENCE LISTING (146 sequences)

SEQ ID NO: 1 - DNA, 20 bp, Artificial Sequence
  Probe or primer sequence
gaaggyccaaarggtggwcc

SEQ ID NO: 2 - DNA, 23 bp, Artificial Sequence
  Probe or primer sequence
gawccaccmgaraatctrccrtc

SEQ ID NO: 3 - DNA, 26 bp, Artificial Sequence
  Probe or primer sequence
gawggyttcaacatttcyggcatacc

SEQ ID NO: 4 - DNA, 18 bp, Artificial Sequence
  Probe or primer sequence
tcccttgttggccgattt

SEQ ID NO: 5 - DNA, 22 bp, Artificial Sequence
  Probe or primer sequence
taccgtcgataatgccttcttt

SEQ ID NO: 6 - DNA, 22 bp, Artificial Sequence
  Probe or primer sequence
aatcttgcagagggtgtcttag

SEQ ID NO: 7 - DNA, 19 bp, Artificial Sequence
  Probe or primer sequence
gggtcgcctgtgacaatag

SEQ ID NO: 8 - DNA, 21 bp, Artificial Sequence
  Probe or primer sequence
cagtaaatagggctggcttga

SEQ ID NO: 9 - DNA, 22 bp, Artificial Sequence
  Probe or primer sequence
ccttctgtaccgttggtgatac

SEQ ID NO: 10 - DNA, 20 bp, Artificial Sequence
  Probe or primer sequence
tgcctcgtcgtttgacatta

SEQ ID NO: 11 - DNA, 21 bp, Artificial Sequence
  Probe or primer sequence
gtacttaccggatggcttgaa

SEQ ID NO: 12 - DNA, 20 bp, Artificial Sequence
  Probe or primer sequence
cattgccactggtggttcta

SEQ ID NO: 13 - DNA, 22 bp, Artificial Sequence
  Probe or primer sequence
gatggcttgaagtcggctaata

SEQ ID NO: 14 - DNA, 20 bp, Artificial Sequence
  Probe or primer sequence
ccaggttctgctgttggtaa

SEQ ID NO: 15 - DNA, 22 bp, Artificial Sequence
  Probe or primer sequence
ccaaagcagtgatgaaggaatg

SEQ ID NO: 16 - DNA, 23 bp, Artificial Sequence
  Probe or primer sequence
gttctgctgttggtaaaatcact

SEQ ID NO: 17 - DNA, 22 bp, Artificial Sequence
  Probe or primer sequence
caaagcagtgatgaaggaatgt

SEQ ID NO: 18 - DNA, 21 bp, Artificial Sequence
  Probe or primer sequence
gccatgggaagacacaataga

SEQ ID NO: 19 - DNA, 23 bp, Artificial Sequence
  Probe or primer sequence
tcttgtcggcaatagctggatta

SEQ ID NO: 20 - DNA, 19 bp, Artificial Sequence
  Probe or primer sequence
gtggtggaagcaagagtaa

SEQ ID NO: 21 - DNA, 22 bp, Artificial Sequence
  Probe or primer sequence
gcttctcttccaaagtgatttg

SEQ ID NO: 22 - DNA, 23 bp, Artificial Sequence
  Probe or primer sequence
ttcaagccatctggtaagtatgt

SEQ ID NO: 23 - DNA, 22 bp, Artificial Sequence
  Probe or primer sequence
cagttaaggcgtcaccgtataa

SEQ ID NO: 24 - DNA, 20 bp, Artificial Sequence
  Probe or primer sequence
taaggccgaatggaaacctc

SEQ ID NO: 25 - DNA, 20 bp, Artificial Sequence
  Probe or primer sequence
acaaccttgggaggcattag

SEQ ID NO: 26 - DNA, 22 bp, Artificial Sequence
  Probe or primer sequence
gcctctcaggctatgttgtatg

SEQ ID NO: 27 - DNA, 20 bp, Artificial Sequence
  Probe or primer sequence
accagaccaccaacaagaac

SEQ ID NO: 28 - DNA, 22 bp, Artificial Sequence
  Probe or primer sequence
gacggtatctcgatgggtacta

SEQ ID NO: 29 - DNA, 21 bp, Artificial Sequence
  Probe or primer sequence
atcgtagtgttgtgccatcat

SEQ ID NO: 30 - DNA, 22 bp, Artificial Sequence
  Probe or primer sequence
atgggttacggcttaggtaaag

SEQ ID NO: 31 - DNA, 22 bp, Artificial Sequence
  Probe or primer sequence
tctggaacaatatggccgatta

SEQ ID NO: 32 - DNA, 22 bp, Artificial Sequence
  Probe or primer sequence
ggtggtatgtacactgccaata

SEQ ID NO: 33 - DNA, 20 bp, Artificial Sequence
  Probe or primer sequence
ccgaaactgctggagatgat

SEQ ID NO: 34 - DNA, 21 bp, Artificial Sequence
  Probe or primer sequence
tggtttcaaggacgaggattt

SEQ ID NO: 35 - DNA, 23 bp, Artificial Sequence
  Probe or primer sequence
cagaatctgcacatgccttattt

SEQ ID NO: 36 - DNA, 20 bp, Artificial Sequence
  Probe or primer sequence
gcggttctcacggtttctta

SEQ ID NO: 37 - DNA, 22 bp, Artificial Sequence
  Probe or primer sequence
ggcaagttcttcttcggataca

SEQ ID NO: 38 - DNA, 22 bp, Artificial Sequence
  Probe or primer sequence
ggaagaggccagagttgaaata

SEQ ID NO: 39 - DNA, 20 bp, Artificial Sequence
  Probe or primer sequence
cctttggaggcatcagagac

SEQ ID NO: 40 - DNA, 22 bp, Artificial Sequence
  Probe or primer sequence
gttcacgacggagatgagattg

SEQ ID NO: 41 - DNA, 22 bp, Artificial Sequence
  Probe or primer sequence
ggagaccacagcttctttcttt

SEQ ID NO: 42 - DNA, 20 bp, Artificial Sequence
  Probe or primer sequence
tcgatcgtgacccaggataa

SEQ ID NO: 43 - DNA, 22 bp, Artificial Sequence
  Probe or primer sequence
gaccaccatacacttccaactc

SEQ ID NO: 44 - DNA, 22 bp, Artificial Sequence
  Probe or primer sequence
gtgacccaggataagtctcaag

SEQ ID NO: 45 - DNA, 20 bp, Artificial Sequence
  Probe or primer sequence
ggtttcctgaccaccataca

SEQ ID NO: 46 - DNA, 22 bp, Artificial Sequence
  Probe or primer sequence
caaggctggattgaaaggaatg

SEQ ID NO: 47 - DNA, 21 bp, Artificial Sequence
  Probe or primer sequence
acatctgggccatcactaaag

SEQ ID NO: 48 - DNA, 23 bp, Artificial Sequence
  Probe or primer sequence
ctcctctgtaggcgttgaaatta

SEQ ID NO: 49 - DNA, 22 bp, Artificial Sequence
  Probe or primer sequence
caagtcagccatcacgtactta

SEQ ID NO: 50 - DNA, 22 bp, Artificial Sequence
  Probe or primer sequence
caccggtaaggaaggaacatac

SEQ ID NO: 51 - DNA, 22 bp, Artificial Sequence
  Probe or primer sequence
ccagcctgtaaagcagtgataa

SEQ ID NO: 52 - DNA, 22 bp, Artificial Sequence
  Probe or primer sequence
gccaagatgttgttggaagaag

SEQ ID NO: 53 - DNA, 22 bp, Artificial Sequence
  Probe or primer sequence
ggcatttgctcaagttctcttt

SEQ ID NO: 54 - DNA, 20 bp, Artificial Sequence
  Probe or primer sequence
aacatgcctggtgtgcttat

SEQ ID NO: 55 - DNA, 22 bp, Artificial Sequence
  Probe or primer sequence
ggcataatggtaccaccgtaaa

SEQ ID NO: 56 - DNA, 24 bp, Artificial Sequence
  Probe or primer sequence
actggtaagacactcaaagagaac

SEQ ID NO: 57 - DNA, 22 bp, Artificial Sequence
  Probe or primer sequence
ggtttcaatgggttggacaaag

SEQ ID NO: 58 - DNA, 24 bp, Artificial Sequence
  Probe or primer sequence
gaggaggactttatcactgcttta

SEQ ID NO: 59 - DNA, 22 bp, Artificial Sequence
  Probe or primer sequence
tctgataacacacacggtcttt

SEQ ID NO: 60 - DNA, 21 bp, Artificial Sequence
  Probe or primer sequence
attgaacaagcactcctcgat

SEQ ID NO: 61 - DNA, 22 bp, Artificial Sequence
  Probe or primer sequence
ccttaaacccagtagcgtacaa

SEQ ID NO: 62 - DNA, 22 bp, Artificial Sequence
  Probe or primer sequence
gcacttctaacagacggaagat

SEQ ID NO: 63 - DNA, 21 bp, Artificial Sequence
  Probe or primer sequence
tgggacaatgtgaccaatcaa

SEQ ID NO: 64 - DNA, 22 bp, Artificial Sequence
  Probe or primer sequence
ggtaaagccatcagacacttga

SEQ ID NO: 65 - DNA, 23 bp, Artificial Sequence
  Probe or primer sequence
acctccagtggcaatgatataag

SEQ ID NO: 66 - DNA, 23 bp, Artificial Sequence
  Probe or primer sequence
catggtttacggtggtaccatta

SEQ ID NO: 67 - DNA, 22 bp, Artificial Sequence
  Probe or primer sequence
ccgtatgattggaaagcagaga

SEQ ID NO: 68 - DNA, 22 bp, Artificial Sequence
  Probe or primer sequence
caacagaattgacttgctcgtg

SEQ ID NO: 69 - DNA, 23 bp, Artificial Sequence
  Probe or primer sequence
cgcaaagtacctctcttgtatct

SEQ ID NO: 70 - DNA, 15 bp, Artificial Sequence
  Probe or primer sequence
scagggtgcttcsca

SEQ ID NO: 71 - DNA, 16 bp, Artificial Sequence
  Probe or primer sequence
tsgcrtcgtaccactg

SEQ ID NO: 72 - DNA, 25 bp, Artificial Sequence
  Probe or primer sequence
cagtatgggtacaaagggwatgmga

SEQ ID NO: 73 - DNA, 20 bp, Artificial Sequence
  Probe or primer sequence
ggtacsaagggwatgcgata

SEQ ID NO: 74 - DNA, 20 bp, Artificial Sequence
  Probe or primer sequence
ctgatgttcgcrtcrtacca

SEQ ID NO: 75 - DNA, 23 bp, Artificial Sequence
  Probe or primer sequence
tcyatcgaraccgtyatgggtgg

SEQ ID NO: 76 - DNA, 22 bp, Artificial Sequence
  Probe or primer sequence
gcgaagaaacggctttgaataa

SEQ ID NO: 77 - DNA, 23 bp, Artificial Sequence
  Probe or primer sequence
ttgtaccagacagacgaaatacc

SEQ ID NO: 78 - DNA, 24 bp, Artificial Sequence
  Probe or primer sequence
acctgcgccttgttcatatcctcc

SEQ ID NO: 79 - DNA, 21 bp, Artificial Sequence
  Probe or primer sequence
gcctcacagaggaggatatga

SEQ ID NO: 80 - DNA, 20 bp, Artificial Sequence
  Probe or primer sequence
atccagcaggtgcatgttac

SEQ ID NO: 81 - DNA, 24 bp, Artificial Sequence
  Probe or primer sequence
ttcgtctgtctggtacaacggcaa

SEQ ID NO: 82 - DNA, 23 bp, Artificial Sequence
  Probe or primer sequence
aggcctcacagaggaggatatga

SEQ ID NO: 83 - DNA, 21 bp, Artificial Sequence
  Probe or primer sequence
gtaccaagggaatgcgatact

SEQ ID NO: 84 - DNA, 21 bp, Artificial Sequence
  Probe or primer sequence
gatactgatgttcgcgtcgta

SEQ ID NO: 85 - DNA, 23 bp, Artificial Sequence
  Probe or primer sequence
tccatcgaaaccgtcatgggtgg

SEQ ID NO: 86 - DNA, 20 bp, Artificial Sequence
  Probe or primer sequence
ctggtgtgccaccgatatta

SEQ ID NO: 87 - DNA, 21 bp, Artificial Sequence
  Probe or primer sequence
attgctgactctgtcggtatc

SEQ ID NO: 88 - DNA, 24 bp, Artificial Sequence
  Probe or primer sequence
agacaccgttcctggctgatttga

SEQ ID NO: 89 - DNA, 23 bp, Artificial Sequence
  Probe or primer sequence
gctataccaaacggaggagatac

SEQ ID NO: 90 - DNA, 19 bp, Artificial Sequence
  Probe or primer sequence
cttcccaggccatgcttta

SEQ ID NO: 91 - DNA, 24 bp, Artificial Sequence
  Probe or primer sequence
accgaggaggacatgaacaaagct

SEQ ID NO: 92 - DNA, 22 bp, Artificial Sequence
  Probe or primer sequence
ctataccaaacggaggagatac

SEQ ID NO: 93 - DNA, 22 bp, Artificial Sequence
  Probe or primer sequence
gttcaagacggcgatgttattc

SEQ ID NO: 94 - DNA, 20 bp, Artificial Sequence
  Probe or primer sequence
gcaacccagttctcctttct

SEQ ID NO: 95 - DNA, 24 bp, Artificial Sequence
  Probe or primer sequence
atgccgttgcgaacactttgtctg

SEQ ID NO: 96 - DNA, 22 bp, Artificial Sequence
  Probe or primer sequence
ggtatgcctgaaatgctcaaac

SEQ ID NO: 97 - DNA, 21 bp, Artificial Sequence
  Probe or primer sequence
ccatgagaacctccgctaaat

SEQ ID NO: 98 - DNA, 24 bp, Artificial Sequence
  Probe or primer sequence
agtttgatcatgggcgctggtcta

SEQ ID NO: 99 - DNA, 22 bp, Artificial Sequence
  Probe or primer sequence
acactttgtctgtggacgtatc

SEQ ID NO: 100 - DNA, 20 bp, Artificial Sequence
  Probe or primer sequence
gtaacagctcgggcgtattt

SEQ ID NO: 101 - DNA, 25 bp, Artificial Sequence
  Probe or primer sequence
tcaaggtcactcacggaacattgct

SEQ ID NO: 102 - DNA, 20 bp, Artificial Sequence
  Probe or primer sequence
cgtcgaaagcggttctatca

SEQ ID NO: 103 - DNA, 22 bp, Artificial Sequence
  Probe or primer sequence
ggtttgagcatttcaggcatac

SEQ ID NO: 104 - DNA, 24 bp, Artificial Sequence
  Probe or primer sequence
tgttgtcttgaggtaccttggccc

SEQ ID NO: 105 - DNA, 23 bp, Artificial Sequence
  Probe or primer sequence
tcgacccagtatgatggtttatg

SEQ ID NO: 106 - DNA, 21 bp, Artificial Sequence
  Probe or primer sequence
tttggccttcttggaggtatc

SEQ ID NO: 107 - DNA, 24 bp, Artificial Sequence
  Probe or primer sequence
cggagaggtgctcgacattgtgtc

SEQ ID NO: 108 - DNA, 16 bp, Artificial Sequence
  Probe or primer sequence
tacgggaggcagcagt

SEQ ID NO: 109 - DNA, 16 bp, Artificial Sequence
  Probe or primer sequence
tattaccgcggctgct

SEQ ID NO: 110 - DNA, 28 bp, Artificial Sequence
  Probe or primer sequence
ccgcagaataagcaccggctaactccgt

SEQ ID NO: 111 - DNA, 30 bp, Artificial Sequence
  Probe or primer sequence
cctaaccagaaagccacggctaactacgtg

SEQ ID NO: 112 - DNA, 19 bp, Artificial Sequence
  Probe or primer sequence
caacgcgaagaaccttacc

SEQ ID NO: 113 - DNA, 18 bp, Artificial Sequence
  Probe or primer sequence
acgtcatccccaccttcc

SEQ ID NO: 114 - DNA, 21 bp, Artificial Sequence
  Probe or primer sequence
acgacaaccatgcaccacctg

SEQ ID NO: 115 - DNA, 20 bp, Artificial Sequence
  Probe or primer sequence
acgacagccatgcagcacct

SEQ ID NO: 116 - DNA, 24 bp, Artificial Sequence
  Probe or primer sequence
cggcggtacacctgctgttatgaa

SEQ ID NO: 117 - DNA, 28 bp, Artificial Sequence
  Probe or primer seqeuence
tgcttgattgtggatgtagcaaatgtcc

SEQ ID NO: 118 - DNA, 24 bp, Artificial Sequence
  Probe or primer sequence
aattgcatggctttcaagccagcc

SEQ ID NO: 119 - DNA, 24 bp, Artificial Sequence
  Probe or primer sequence
attagccgacttcaagccatccgg

SEQ ID NO: 120 - DNA, 26 bp, Artificial Sequence
  Probe or primer sequence
tgcctcgtcgtttgacattaccatca

SEQ ID NO: 121 - DNA, 30 bp, Artificial Sequence
  Probe or primer sequence
aggtacttacttcaagggtaaagctagagt

SEQ ID NO: 122 - DNA, 25 bp, Artificial Sequence
  Probe or primer sequence
tactatcttgccagggtctcccaca

SEQ ID NO: 123 - DNA, 24 bp, Artificial Sequence
  Probe or primer sequence
tcctgctattgctgacaagattga

SEQ ID NO: 124 - DNA, 24 bp, Artificial Sequence
  Probe or primer sequence
aggtcggtggtactcaaagtgtca

SEQ ID NO: 125 - DNA, 26 bp, Artificial Sequence
  Probe or primer sequence
tgctaagttagtctctaatgcctccc

SEQ ID NO: 126 - DNA, 24 bp, Artificial Sequence
  Probe or primer sequence
tcagcaaagcgcaagttggtgttg

SEQ ID NO: 127 - DNA, 30 bp, Artificial Sequence
  Probe or primer sequence
agagaaattatcgcagactctttcgagact

SEQ ID NO: 128 - DNA, 24 bp, Artificial Sequence
  Probe or primer sequence
attctctggcggttctcacggttt

SEQ ID NO: 129 - DNA, 24 bp, Artificial Sequence
  Probe or primer sequence
acccaagacttctgaggctgaagc

SEQ ID NO: 130 - DNA, 24 bp, Artificial Sequence
  Probe or primer sequence
tggttcttgctggtggtctggaaa

SEQ ID NO: 131 - DNA, 24 bp, Artificial Sequence
  Probe or primer sequence
agaagctttcgaaggcggtccaat

SEQ ID NO: 132 - DNA, 26 bp, Artificial Sequence
  Probe or primer sequence
cctctcttatatttcaactctggcct

SEQ ID NO: 133 - DNA, 26 bp, Artificial Sequence
  Probe or primer sequence
tgcttgtctcggaagaaattctcgct

SEQ ID NO: 134 - DNA, 24 bp, Artificial Sequence
  Probe or primer sequence
tgcttcgcaagccatgttgtatgc

SEQ ID NO: 135 - DNA, 27 bp, Artificial Sequence
  Probe or primer sequence
accgttggtgataccatcagaaactcc

SEQ ID NO: 136 - DNA, 24 bp, Artificial Sequence
  Probe or primer sequence
tctgacagcactccattgttggct

SEQ ID NO: 137 - DNA, 24 bp, Artificial Sequence
  Probe or primer sequence
aaaggctagagtgtttgacgccga

SEQ ID NO: 138 - DNA, 25 bp, Artificial Sequence
  Probe or primer sequence
tgcttgatggtgaccagatgactgt

SEQ ID NO: 139 - DNA, 25 bp, Artificial Sequence
  Probe or primer sequence
aaggtctgttgtgtctacccatggc

SEQ ID NO: 140 - DNA, 22 bp, Artificial Sequence
  Probe or primer sequence
atatcctggccctcaggcaagc

SEQ ID NO: 141 - DNA, 24 bp, Artificial Sequence
  Probe or primer sequence
tcgcccttcttgatttctccagcc

SEQ ID NO: 142 - DNA, 22 bp, Artificial Sequence
  Probe or primer sequence
tgtcaccgaggatgtgtcgcag

SEQ ID NO: 143 - DNA, 23 bp, Artificial Sequence
  Probe or primer sequence
tttctggtggatcccacggtttc

SEQ ID NO: 144 - DNA, 27 bp, Artificial Sequence
  Probe or primer sequence
cccgtgacatcatgaccaagaaatcgt

SEQ ID NO: 145 - DNA, 24 bp, Artificial Sequence
  Probe or primer sequence
tggccatgggtagacacaacagac

SEQ ID NO: 146 - DNA, 24 bp, Artificial Sequence
  Probe or primer sequence
aagaaagaagtggctgcctcctga

(19) United States
(12) Patent Application Publication
(10) Pub. No.: US 2022/0162712 A1
(43) Pub. Date: May 26, 2022

(54) DETECTION AND DELINEATION OF MICROORGANISMS

(71) Applicant: MOMENTUM BIOSCIENCE LIMITED, St Mellons, Cardiff (GB)

(72) Inventor: Andrew Rogers, St. Mellons, Cardiff (GB)

(73) Assignee: MOMENTUM BIOSCIENCE LIMITED, St Mellons, Cardiff (GB)

(21) Appl. No.: 16/604,956

(22) PCT Filed: Apr. 11, 2018
(86) PCT No.: PCT/GB2018/000065

§ 371 Date: Oct. 11, 2019

(30) Foreign Application Priority Data
Apr. 12, 2017 (GB) 1705932.0
Jul. 25, 2017 (GB) 1711949.6

(51) Int. Cl. C12Q 1/6895

(52) U.S. Cl. CPC C12Q 1/6895; C12Q 2600/16; C12Q 2600/158

(57) ABSTRACT

Methods for detecting a fungal/yeast infection in a sample, comprising: performing a nucleic acid amplification reaction to amplify the ILV3 gene of fungi/yeast; and detecting the amplification product to determine whether the sample contains a fungal/yeast infection. Corresponding primers, probes and kits are also provided.

### CROSS-REFERENCE TO RELATED APPLICATIONS

The present application is the U.S. National Stage under 35 U.S.C. § 371 of International Application No. PCT/GB2018/000065, filed on Apr. 11, 2018, which claims the benefit of priority under 35 U.S.C. § 119(e) to Great Britain Application No. 1705932.0, filed on Apr. 12, 2017, and Great Britain Application No. 1711949.6, filed on Jul. 25, 2017, the contents of which are incorporated herein by reference in their entireties.

### REFERENCE TO SEQUENCE LISTING AS TEXT FILE

The present application was filed with a Sequence Listing on Oct. 11, 2019, which was submitted electronically in ASCII format and is hereby incorporated by reference in its entirety. The ASCII copy of the Sequence Listing, created on Oct. 11, 2019, is named “Sequence Listing” and is 28.4 KB (29,129 bytes) in size.

### FIELD OF THE INVENTION

The invention provides a novel target in the context of detecting whether a fungus or yeast is present in a sample. This target, ILV3, encodes a dihydroxyacid dehydratase and is particularly useful in clinical diagnostic applications due to the lack of any sequence identity with the human genome. Primers and probes are provided which avow the presence or absence of Candida, Aspergillus and Cryptococcus neoformans to be determined in a sample. Once the presence of a fungus or yeast is determined, the identity of that species is usefully obtained, for example to direct therapy. As well as pan-Candida and pan-Aspergillus detection using a single primer/probe set there are also provided species-specific primer pairs that enable the species of Candida or Aspergillus to be determined in the sample. Melt curve analysis may be employed as a method of determining which species is present in the sample. The methods may be combined with methods that determine whether bacteria are present in the sample and which may categorise those bacteria as either Gram positive or Gram negative.

### BACKGROUND OF THE INVENTION

The US centre for disease control estimates that at least 30% of patients are prescribed antibiotics unnecessarily (Journal of the American Medical Association, May 2016). Antibiotic resistance is a pressing public health threat. Every year in the UK there are over 150,000 cases of sepsis resulting in 44,000 deaths. Many of these deaths are due to antibiotic resistant microbial infections. Culture-based identification of microbial infections takes around 5 days on average, during which time antibiotics are administered. It would be of great benefit if a more rapid test could be developed in order to detect a microbial infection in a sample.

Some molecular tests for identifying microbial DNA or RNA are known. For example, U.S. Pat. Nos. 7,291,724 and 7,169,555 describe oligonucleotides binding to ribosomal DNA that can be used in PCR reactions. Further amplification based methods for the detection of fungus/yeast are known. For example, see WO2002/27021, CN105018575, U.S. Pat. No. 6,872,523 and Van Burk et al., 1998.

### DESCRIPTION OF THE INVENTION

Fungal infections (fungaemia) of the blood are highly dangerous. The inventors have investigated molecular targets that may be probed to rapidly (same day results) identify a fungal infection. The invention thus provides ILV3 as a novel target in the context of detecting whether a fungus or yeast is present in a sample. ILV3 encodes a dihydroxyacid dehydratase that catalyses the third step hi the common pathway leading to biosynthesis of branched-chain amino acids. The inventors have discovered that this gene is present in the most clinically relevant fungal species and can be specifically targeted to permit detection and identification of a fungal infection. Moreover, this target is particularly useful in clinical diagnostic applications due to the lack of any sequence identity with the human genome. In more detail, Liu et al., 2006 lists a number of candidate genes as potential targets for antifungal drug discovery, Of all the candidates listed, only ILV3 has a 0% identity with humans at the protein level. After conducting further analysis, the inventors have found that ILV3 also has no homology with humans or bacteria (of either Gram positive or Gram negative status) at the genetic level. This contrasts with the other candidate genes disclosed in Liu et al., in which the inventors found genetic homology to humans. For example, ILV5 (2% identity with humans at the protein level) resulted in two positive nucleotide alignments (82% identity) with the human genome. Thus, candidate genes other than ILV3 (including ribosomal candidates, e.g. 18S, 28S or 5.8S) may result in the false positive detection and identification of a fungal infection in samples comprising human genetic material. Thus, in its broadest aspect, the invention provides for the use of ILV3 to identify a fungal infection in a sample.

It should be noted that throughout the specification the term “comprising” is intended to represent open-ended (i.e. including) language. However, for the avoidance of doubt, wherever the term “comprising” is used it is envisaged that the corresponding feature may be limited to that specified (i.e. consisting) as necessary.

Accession numbers and related information for the ILV3 gene sequence in the species of interest are provided in Table A:

| | Reference | Accession | | | Orientation of |
|---|---|---|---|---|---|
| Organism | strain | number | Gene ID | Gene symbol | gene |
| Candida albicans | SC5314 | NC_032093.1 | 3636428 | ILV3 | Anti-sense |
| Candida glabrata | CBS138 | NC_005968.1 | 2886651 | CAGL0B03993g | Anti-sense |
| Candida | CDC317 | HE605203.1 | CPAR2_100130 | CPAR2_100130 | Anti-sense |
| parapsilosis | | | (locus_tag) | | |
| Candida tropicalis | MYA3404 | NW_03020040.1 | 8300008 | CTRG_06147 | Anti-sense |
| Candida krusei | SD108 | JQFK01000016.1 | JL09_g2096 | JL09_g2096 | Sense |
| | | | (locus tag) | | |
| Candida | CD36 | NC_012864.1 | 8048016 | CD36_55010 | Anti-sense |
| dubliniensis | | | | | |
| Candida | ATCC 6260 | NW_001809800.1 | 5129681 | PGUG_00520 | Anti-sense |
| guilliermondii | | | | | |
| Candida auris | 6684 | NW_017263971.1 | 28879407 | QG37_05711 | Anti-sense |
| Aspergillus | Af293 | NC_007195.1 | 3512998 | AFUA_2G14210 | Sense |
| fumigatus | | | | | |
| Aspergillus niger | CBS 513.88 | NT_166533.1 | 4989816 | ANI_1_1182164 | Anti-sense |
| Aspergillus flavus | NRRL3357 | NW_002477240.1 | 7922100 | AFLA_105610 | Anti-sense |
| Cryptococcus | JEC21 | NC_006693.1 | 3259119 | CNH01530 | Sense |
| neoformans | | | | | |

Through extensive characterisation and testing, the inventors have identified specific regions of the ILV3 gene that can be targeted. Firstly, the inventors have identified regions of the ILV3 gene in 8 clinically prevalent Candida species that can be commonly targeted using a single primer and/or probe set. A single primer and/or probe set specifically hybridises to ILV3 from these 8 species but does not cross-react with the ILV3 gene from non-Candida species. Such primer/probes may be referred to as “pan-Candida”.

Secondly, the inventors have identified regions of the ILV3 gene in 3 clinically prevalent Aspergillus species that can be commonly targeted using a single primer and/or probe set. A single primer and/or probe set specifically hybridises to these 3 species but does not cross-react with the ILV3 gene from non-Aspergillus species. Such primer/probes may be referred to as “pan-Aspergillus”.

Thirdly, the inventors have identified regions of the ILV3 gene in Cryptococcus neoformans that can be targeted using a primer and/or probe set. A primer and/or probe set specifically hybridises to this species but does not cross-react with the ILV3 gene from non-Cryptococcus species.

Collectively, the first, second and third category of target regions can be probed to determine whether there is a fungal infection in the sample. With appropriate discrimination of amplification products it can be determined which category of fungal infection is present.

Fourthly, the inventors have identified regions of the ILV3 gene that differ between 8 clinically prevalent Candida species that can thus each be separately targeted using suitably designed primer and/or probe sets. Each primer and/or probe set specifically hybridises to one Candida species but does not cross-react with the ILV3 gene from other Candida species (or non-Candida species).

Fifthly, the inventors have identified differing regions of the ILV3 gene in 3 clinically prevalent Aspergillus species that can thus each be separately targeted using suitably designed primer and/or probe sets. Each primer and/or probe set specifically hybridises to one Aspergillus species but does not cross-react with the ILV3 gene from other Aspergillus species (or non-Aspergillus species).

The fourth and fifth category of target regions can be probed to more specifically identify the nature of a fungal infection in the sample. With appropriate detection of amplification products the species responsible for the infection can be identified. This may facilitate treatment.

Specific primer and probe sequences are provided that target the respective regions and which also form an aspect of the present invention.

Table B below identifies the various ILV3 target regions, identified with reference to the sequences provided In Table A. Table B also provides specific primer and probe sequences of the invention that target the respective regions, together with the SEQ ID NO used in the sequence listing for each sequence:

| | | Forward primer | Forward primer | | Reverse primer | Reverese primer | | Probe | Probe |
|---|---|---|---|---|---|---|---|---|---|
| Name | SEQ ID. | sequence⁽ⁱ⁾ | location⁽ⁱⁱ⁾ | SEQ ID. | sequence⁽ⁱ⁾ | location⁽ⁱⁱ⁾ | SEQ ID. | sequence⁽ⁱ⁾ | location⁽ⁱⁱ⁾ |
| Pan- | 1 | GAAGGYCC | CA: | 2 | GAWCCACC | CA: | 3 | GAWGGY | CA: |
| Candida | | AAARGGTG | c1169825-1169806 | | MGARAATC | 1169707-1169729 | | TTCAACAT | 1169779-1169804 |
| (ILV3) | | GWCC | CGI: | | TRCCRTC | CGI: | | TTCYGGC | CGI: |
| | | | c393798-393779 | | | 393680-393702 | | ATACC | 393752-393777 |
| | | | CT: | | | CT: | | | CT: |
| | | | c405996-405978 | | | 405878-405900 | | | 405950-405975 |
| | | | CDu: | | | CDu: | | | CDu: |
| | | | c1218036-1218017 | | | 1217918-1217940 | | | 1217990-1218015 |
| | | | CGu: | | | CGu: | | | CGu: |
| | | | c909809-909790 | | | 909691-909713 | | | 909763-909788 |
| | | | CP: | | | CP: | | | CP: |
| | | | c24006-23987 | | | 23888-23910 | | | 23960-23985 |
| | | | CK: | | | CK: | | | CK: |
| | | | 61666-61685 | | | c61784-61762 | | | c61712-61687 |
| | | | CAu: | | | CAu: | | | CAu: |
| | | | c32324-32305 | | | 32206-32228 | | | 32278-32303 |
| Candida | 4 | TCCCTTGTT | c1170234-1170217 | 5 | TACCGTCGA | 1170119-1170140 | 116 | CGGCGGT | c1170174-1170151 |
| albicans - | | GGCCGATTT | | | TAATGCCTT | | | ACACCTG | |
| melt | | | | | CTTT | | | CTGTTAT | |
| (ILV3) | | | | | | | | GAA | |
| | 6 | AATCTTGCA | c1171213-1171192 | 7 | GGGTCGCCT | 1171120-1171138 | 117 | TGCTTGA | 1171165-1171192 |
| | | GAGGGTGT | | | GTGACAATA | | | TTGTGGA | |
| | | CTTAG | | | G | | | TGTAGCA | |
| | | | | | | | | AATGTCC | |
| Candida | 8 | CAGTAAATA | c1219181-1219161 | 9 | CCTTCTGTA | 1219103-1219124 | 118 | AATTGCA | 1219148-1219171 |
| dubliniensis - | | GGGCTGGC | | | CCGTTGGTG | | | TGGCTTTC | |
| melt | | TTGA | | | ATAC | | | AAGCCAG | |
| (ILV3) | | | | | | | | CC | |
| | 10 | TGCCTCGTC | c1218505-1218486 | 11 | GTACTTACC | 1218409-1218429 | 119 | ATTAGCC | c1218439-1218416 |
| | | GTTTGACAT | | | GGATGGCTT | | | GACTTCA | |
| | | TA | | | GAA | | | AGCCATC | |
| | | | | | | | | CGG | |
| | 12 | CATTGCCAC | c1218553-1218534 | 13 | GATGGCTTG | 1218419-1218440 | 120 | TGCCTCG | c1218505-1218480 |
| | | TGGTGGTTC | | | AAGTCGGCT | | | TCGTTTG | |
| | | TA | | | AATA | | | ACATTACC | |
| | | | | | | | | ATCA | |
| Candida | 14 | CCAGGTTCT | c406146-406127 | 15 | CCAAAGCA | 406047-406068 | 121 | AGGTACT | c406111-406082 |
| tropicalis - | | GCTGTTGGT | | | GTGATGAA | | | TACTTCAA | |
| melt | | AA | | | GGAATG | | | GGGTAAA | |
| (ILV3) | | | | | | | | GCTAGAG | |
| | | | | | | | | T | |
| | 16 | GTTCTGCTG | c406142-406120 | 17 | CAAAGCAG | 406048-406069 | 121 | AGGTACT | c406111-406082 |
| | | TTGGTAAAA | | | TGATGAAG | | | TACTTCAA | |
| | | TCACT | | | GAATGT | | | GGGTAAA | |
| | | | | | | | | GCTAGAG | |
| | | | | | | | | T | |
| Candida | 18 | GCCATGGG | c24951-24931 | 19 | TCTTGTCGG | 24843-24865 | 122 | TACTATCT | c24907-24883 |
| parapsilosis - | | AAGACACA | | | CAATAGCTG | | | TGCCAGG | |
| melt | | ATAGA | | | GATTA | | | GTCTCCC | |
| (ILV3) | | | | | | | | ACA | |
| | 20 | GTGGTGGA | c24881-24863 | 21 | GCTTCTCTT | 24774-24795 | 123 | TCCTGCTA | c24862-24839 |
| | | AGCAAGAG | | | CCAAAGTG | | | TTGCTGA | |
| | | TAA | | | ATTTG | | | CAAGATT | |
| | | | | | | | | GA | |
| Candida | 22 | TTCAAGCCA | c394191-394169 | 23 | CAGTTAAG | 394080-394101 | 124 | AGGTCGG | c394151-394128 |
| glabrata - | | TCTGGTAAG | | | GCGTCACCG | | | TGGTACT | |
| melt | | TATGT | | | TATAA | | | CAAAGTG | |
| (ILV3) | | | | | | | | TCA | |
| | 24 | TAAGGCCG | c393535-393516 | 25 | ACAACCTTG | 393445-393464 | 125 | TGCTAAG | c393478-393453 |
| | | AATGGAAA | | | GGAGGCAT | | | TTAGTCTC | |
| | | CCTC | | | TAG | | | TAATGCC | |
| | | | | | | | | TCCC | |
| | 26 | GCCTCTCAG | c395076-395055 | 27 | ACCAGACCA | 394987-395006 | 126 | TCAGCAA | c395030-395007 |
| | | GCTATGTTG | | | CCAACAAG | | | AGCGCAA | |
| | | TATG | | | AAC | | | GTTGGTG | |
| | | | | | | | | TTG | |
| | 28 | GACGGTATC | c394884-394863 | 29 | ATCGTAGTG | 394783-394803 | 127 | AGAGAAA | c394836-394807 |
| | | TCGATGGGT | | | TTGTGCCAT | | | TTATCGC | |
| | | ACTA | | | CAT | | | AGACTCT | |
| | | | | | | | | TTCGAGA | |
| | | | | | | | | CT | |
| Candida | 30 | ATGGGTTAC | 61723-61744 | 31 | TCTGGAACA | c61817-61796 | 128 | ATTCTCTG | 61770-61793 |
| krusei - | | GGCTTAGGT | | | ATATGGCCG | | | GCGGTTC | |
| melt | | AAAG | | | ATTA | | | TCACGGT | |
| (ILV3) | | | | | | | | TT | |
| | 32 | GGTGGTAT | 60940-60961 | 33 | CCGAAACTG | c61030-61011 | 129 | ACCCAAG | c60990-60967 |
| | | GTACACTGC | | | CTGGAGAT | | | ACTTCTG | |
| | | CAATA | | | GAT | | | AGGCTGA | |
| | | | | | | | | AGC | |
| | 34 | TGGTTTCAA | 60420-60440 | 35 | CAGAATCTG | c60535-60513 | 130 | TGGTTCTT | 60462-60485 |
| | | GGACGAGG | | | CACATGCCT | | | GCTGGTG | |
| | | ATTT | | | TATTT | | | GTCTGGA | |
| | | | | | | | | AA | |
| | 36 | GCGGTTCTC | 61778-61797 | 37 | GGCAAGTTC | c61923-61902 | 131 | AGAAGCT | 61815-61838 |
| | | ACGGTTTCT | | | TTCTTCGGA | | | TTCGAAG | |
| | | TA | | | TACA | | | GCGGTCC | |
| | | | | | | | | AAT | |
| | 38 | GGAAGAGG | 61940-61961 | 39 | CCTTTGGAG | c62015-61996 | 132 | CCTCTCTT | c61970-61945 |
| | | CCAGAGTTG | | | GCATCAGA | | | ATATTTCA | |
| | | AAATA | | | GAC | | | ACTCTGG | |
| | | | | | | | | CCT | |
| Candida | 40 | GTTCACGAC | c909629-909608 | 41 | GGAGACCA | 909529-909550 | 133 | TGCTTGTC | c909577-909552 |
| guilliermondii - | | GGAGATGA | | | CAGCTTCTT | | | TCGGAAG | |
| melt | | GATTG | | | TCTTT | | | AAATTCTC | |
| (ILV3) | | | | | | | | GCT | |
| | 42 | TCGATCGTG | c911117-911098 | 43 | GACCACCAT | 911002-911023 | 134 | TGCTTCG | c911088-911065 |
| | | ACCCAGGAT | | | ACACTTCCA | | | CAAGCCA | |
| | | AA | | | ACTC | | | TGTTGTAT | |
| | | | | | | | | GC | |
| | 44 | GTGACCCA | c911111-911090 | 45 | GGTTTCCTG | 910994-911013 | 134 | TGCTTCG | c911088-911065 |
| | | GGATAAGT | | | ACCACCATA | | | CAAGCCA | |
| | | CTCAAG | | | CA | | | TGTTGTAT | |
| | | | | | | | | GC | |
| | 46 | CAAGGCTG | c910941-910920 | 47 | ACATCTGGG | 910799-910819 | 135 | ACCGTTG | 910878-910904 |
| | | GATTGAAA | | | CCATCACTA | | | GTGATAC | |
| | | GGAATG | | | AAG | | | CATCAGA | |
| | | | | | | | | AACTCC | |
| Candida | 48 | CTCCTCTGT | c32790-32768 | 49 | CAAGTCAGC | 32682-32703 | 136 | TCTGACA | c32744-32721 |
| auris - | | AGGCGTTG | | | CATCACGTA | | | GCACTCC | |
| melt | | AAATTA | | | CTTA | | | ATTGTTG | |
| (ILV3) | | | | | | | | GCT | |
| | 50 | CACCGGTAA | c32451-32430 | 51 | CCAGCCTGT | 32368-32389 | 137 | AAAGGCT | c32421-32398 |
| | | GGAAGGAA | | | AAAGCAGT | | | AGAGTGT | |
| | | CATAC | | | GATAA | | | TTGACGC | |
| | | | | | | | | CGA | |
| | 52 | GCCAAGAT | c32654-32633 | 53 | GGCATTTGC | 32567-32588 | 138 | TGCTTGA | c32629-32605 |
| | | GTTGTTGGA | | | TCAAGTTCT | | | TGGTGAC | |
| | | AGAAG | | | CTTT | | | CAGATGA | |
| | | | | | | | | CTGT | |
| | 54 | AACATGCCT | c33278-33259 | 55 | GGCATAAT | 33202-33223 | 139 | AAGGTCT | 33233-33257 |
| | | GGTGTGCTT | | | GGTACCACC | | | GTTGTGT | |
| | | AT | | | GTAAA | | | CTACCCAT | |
| | | | | | | | | GGC | |
| | 56 | ACTGGTAA | c32603-32580 | 57 | GGTTTCAAT | 32512-32533 | 140 | ATATCCT | 32542-32563 |
| | | GACACTCAA | | | GGGTTGGA | | | GGCCCTC | |
| | | AGAGAAC | | | CAAAG | | | AGGCAAG | |
| | | | | | | | | C | |
| | 58 | GAGGAGGA | c32399-32376 | 59 | TCTGATAAC | 32328-32349 | 141 | TCGCCCTT | 32350-32373 |
| | | CTTTATCAC | | | ACACACGGT | | | CTTGATTT | |
| | | TGCTTTA | | | CTTT | | | CTCCAGC | |
| | | | | | | | | C | |
| | 60 | ATTGAACAA | c33648-33628 | 61 | CCTTAAACC | 33566-33587 | 142 | TGTCACC | c33627-33606 |
| | | GCACTCCTC | | | CAGTAGCGT | | | GAGGATG | |
| | | GAT | | | ACAA | | | TGTCGCA | |
| | | | | | | | | G | |
| | 62 | GCACTTCTA | c32240-32219 | 63 | TGGGACAA | 32175-32195 | 143 | TTTCTGGT | c32218-32196 |
| | | ACAGACGG | | | TGTGACCAA | | | GGATCCC | |
| | | AAGAT | | | TCAA | | | ACGGTTT | |
| | | | | | | | | C | |
| | 64 | GGTAAAGC | c32930-32909 | 65 | ACCTCCAGT | 32826-32848 | 144 | CCCGTGA | c32890-32864 |
| | | CATCAGACA | | | GGCAATGA | | | CATCATG | |
| | | CTTGA | | | TATAAG | | | ACCAAGA | |
| | | | | | | | | AATCGT | |
| | 66 | CATGGTTTA | c33228-33206 | 67 | CCGTATGAT | 33130-33151 | 145 | TGGCCAT | c33259-33236 |
| | | CGGTGGTA | | | TGGAAAGC | | | GGGTAGA | |
| | | CCATTA | | | AGAGA | | | CACAACA | |
| | | | | | | | | GAC | |
| | 68 | CAACAGAAT | c32106-32085 | 69 | CGCAAAGT | 32011-32033 | 146 | AAGAAAG | c32061-32038 |
| | | TGACTTGCT | | | ACCTCTCTT | | | AAGTGGC | |
| | | CGTG | | | GTATCT | | | TGCCTCCT | |
| | | | | | | | | GA | |
| Pan- | 70 | SCAGGGTG | AFu: | 71 | TSGCRTCGT | AFu: | 72 | CAGTATG | AFu: |
| Aspergillus V1 | | CTTCSCA | 3721583-3721597 | | ACCACTG | c3721887-3721872 | | GGTACAA | 3721790-3721814 |
| (ILV3) | | | AN: | | | AN: | | AGGGWA | AN: |
| | | | c541018-541004 | | | 540714-540729 | | TGMGA | c540811-540788 |
| | | | AFI: | | | AFI: | | | AFI: |
| | | | c382612-382598 | | | 382308-382323 | | | c382405-382381 |
| Pan- | 73 | GGTACSAA | AFu: | 74 | CTGATGTTC | AFu: | 75 | TCYATCG | AFu: |
| Aspergillus V2 | | GGGWATGC | 3721797-3721816 | | GCRTCRTAC | c3721875-3721894 | | ARACCGT | 3721848-3721870 |
| (ILV3) | | GATA | AN: | | CA | AN: | | YATGGGT | AN: |
| | | | c540804-540785 | | | 540707-540726 | | GG | c540753-540731 |
| | | | AFI: | | | AFI: | | | AFu: |
| | | | c382398-382379 | | | 382301-382320 | | | c382347-382325 |
| Aspergillus | 76 | GCGAAGAA | 3721531-3721552 | 77 | TTGTACCAG | c3721653-3721675 | 78 | ACCTGCG | c3721651-3721628 |
| fumigatus | | ACGGCTTTG | | | ACAGACGA | | | CCTTGTTC | |
| (ILV3) | | AATAA | | | AATACC | | | ATATCCTC | |
| | | | | | | | | C | |
| | 79 | GCCTCACAG | 3721618-3721638 | 80 | ATCCAGCAG | c3721706-3721681 | 81 | TTCGTCTG | 3721658-3721681 |
| | | AGGAGGAT | | | GTGCATGTT | | | TCTGGTA | |
| | | ATGA | | | AC | | | CAACGGC | |
| | | | | | | | | AA | |
| | 82 | AGGCCTCAC | 3721616-3721638 | 80 | ATCCAGCAG | c3721706-3721681 | 81 | TTCGTCTG | 3721658-3721681 |
| | | AGAGGAGG | | | GTGCATGTT | | | TCTGGTA | |
| | | ATATGA | | | AC | | | CAACGGC | |
| | | | | | | | | AA | |
| | 83 | GTACCAAG | 3721798-3721818 | 84 | GATACTGAT | c3721898-3721878 | 85 | TCCATCG | 3721848-3721870 |
| | | GGAATGCG | | | GTTCGCGTC | | | AAACCGT | |
| | | ATACT | | | GTA | | | CATGGGT | |
| | | | | | | | | GG | |
| Aspergillus | 87 | ATTGCTGAC | c540219-540199 | 86 | CTGGTGTGC | 540084-540103 | 88 | AGACACC | c540161-540138 |
| niger | | TCTGTCGGT | | | CACCGATAT | | | GTTCCTG | |
| (ILV3) | | ATC | | | TA | | | GCTGATT | |
| | | | | | | | | TGA | |
| Aspergillus | 90 | CTTCCCAGG | c382604-382586 | 89 | GCTATACCA | 382519-382541 | 91 | ACCGAGG | c382572-382549 |
| flavus | | CCATGCTTT | | | AACGGAGG | | | AGGACAT | |
| (ILV3) | | A | | | AGATAC | | | GAACAAA | |
| | | | | | | | | GCT | |
| | 90 | CTTCCCAGG | c382604-382586 | 92 | CTATACCAA | 382520-382541 | 91 | ACCGAGG | c382572-382549 |
| | | CCATGCTTT | | | ACGGAGGA | | | AGGACAT | |
| | | A | | | GATAC | | | GAACAAA | |
| | | | | | | | | GCT | |
| Cryptococcus | 93 | GTTCAAGAC | 702696-702717 | 94 | GCAACCCA | c702796-702777 | 95 | ATGCCGT | 702724-702747 |
| neoformans | | GGCGATGTT | | | GTTCTCCTT | | | TGCGAAC | |
| (ILV3) | | ATTC | | | TCT | | | ACTTTGTC | |
| | | | | | | | | TG | |
| | 96 | GGTATGCCT | 702499-702520 | 97 | CCATGAGA | c702602-702582 | 98 | AGTTTGA | 702526-702549 |
| | | GAAATGCTC | | | ACCTCCGCT | | | TCATGGG | |
| | | AAAC | | | AAAT | | | CGCTGGT | |
| | | | | | | | | CTA | |
| | 99 | ACACTTTGT | 702736-702757 | 100 | GTAACAGCT | c702850-702831 | 101 | TCAAGGT | 702805-702829 |
| | | CTGTGGAC | | | CGGGCGTA | | | CACTCAC | |
| | | GTATC | | | TTT | | | GGAACAT | |
| | | | | | | | | TGCT | |
| | 102 | CGTCGAAA | 702384-702403 | 103 | GGTTTGAGC | c702521-702500 | 104 | TGTTGTCT | 702423-702446 |
| | | GCGGTTCTA | | | ATTTCAGGC | | | TGAGGTA | |
| | | TCA | | | ATAC | | | CCTTGGC | |
| | | | | | | | | CC | |
| | 105 | TCGACCCAG | 701384-701406 | 106 | TTTGGCCTT | c701499-701479 | 107 | CGGAGAG | 701435-701458 |
| | | TATGATGGT | | | CTTGGAGGT | | | GTGCTCG | |
| | | TTATG | | | ATC | | | ACATTGT | |
| | | | | | | | | GTC | |

(i) All sequences shown are in the 5′ to 3′ orientation
(ii) Primer/probe location is based on the orientation of the gene (c = complement)

In Table B of GB1705932.0, the forward and reverse primer sequences and locations for Aspergillus niger (ILV3) and Aspergillus flavus (ILV3) were incorrectly interchanged and thus recited as follows:

| | | | | | | | | | |
|---|---|---|---|---|---|---|---|---|---|
| Aspergillus | 86 | CTGGTGTGC | 540084-540103 | 87 | ATTGCTGAC | c540219-540199 | 88 | AGACACC | c540161-540138 |
| niger | | CACCGATAT | | | TCTGTCGGT | | | GTTCCTG | |
| (ILV3) | | TA | | | ATC | | | GCTGATT | |
| | | | | | | | | TGA | |
| Aspergillus | 89 | GCTATACCA | 382519-382541 | 90 | CTTCCCAGG | c382604-382586 | 91 | ACCGAGG | c382572-382549 |
| flavus | | AACGGAGG | | | CCATGCTTT | | | AGGACAT | |
| (ILV3) | | AGATAC | | | A | | | GAACAAA | |
| | | | | | | | | GCT | |
| | 92 | CTATACCAA | 382520-382541 | 90 | CTTCCCAGG | C382604-382586 | 91 | ACCGAGG | c382572-382549 |
| | | ACGGAGGA | | | CCATGCTTT | | | AGGACAT | |
| | | GATAC | | | A | | | GAACAAA | |
| | | | | | | | | GCT | |

The designation of the terms “forward” and “reverse” primers is relative to the orientation of the ILV3 gene which is fixed for each of nucleotide sequence accession numbers NT_166533.1 (Aspergillus niger) and NW 002477240.1 (Aspergillus flavus). Thus, the skilled person consulting nucleotide sequence accession numbers NT_166533.1 and NW_002477240.1 when seeking to understand the invention, would immediately and unambiguously realise that the “forward” primer sequence and location as recited in Table B of GB1705932.0 is, in fact, the reverse primer sequence and location and vice versa for Aspergillus niger (ILV3) and Aspergillus flavus (ILV3). This correction has been applied throughout the present application.

Thus, the invention provides primers and probes useful in fungal detection. As would be readily understood by the skilled person, primers and probes hybridise to particular sub-regions within the gene of interest (ILV3). While the primers are specified individually herein, it would be immediately appreciated, based in particular on the information provided in Table B which primers are preferably paired according to the invention, including when defined by reference to their target region. Using the information provided herein, in particular the new target and specific target sequences, primers and probes may be designed by one skilled in the art. Typically, primers are between 15 and 40, such as between 18 and 35, nucleotides in length. Probes are typically between 15 and 100, such as between 20 and 40, nucleotides in length. Some mismatches to the target sequences may be tolerated provided that specific hybridisation is achieved. Specific hybridisation is a term of art well understood by the skilled person to exclude hybridisation to non-target sequences. The skilled person is also aware of suitable reaction conditions used for performing nucleic acid amplification under which specific hybridisation must occur. Moreover, for primers and/or probes which hybridise to multiple target sequences there may be some degeneracy in specific positions. For example, a primer may include any pyrimidine nucleotide (t/u or c) at a given position or a mixture of primers containing at least two of these nucleotides may be adopted. Variants of the specific primers and probes described herein (e.g. by SEQ ID NO) are also envisaged, They may contain nucleotide additions, deletions and/or substitutions provided that specific hybridisation is still achieved. 1, 2, 3, 4, 5, 6 or 7 additions, deletions and/or substitutions may be tolerated in some circumstances. As explained further herein, primers and/or probes may be labelled according to the detection methodology employed. Typical labels are fluorescent molecules, which may be arranged as fluorophores and quenchers in some aspects.

The invention therefore provides at least one primer pair for detecting a yeast/fungus infection in a sample comprising a forward and reverse primer hybridizing specifically to the ILV3 gene of the following Candida species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris

By “hybridising specifically”, or equivalent language, is meant that the primers hybridise to ILV3 from these 8 species but do not hybridise (or cross-react) with the ILV3 gene from non-Candida species. Thus, an amplification product will only be generated if a Candida species (from those 8 species) is present in the sample.

According to some embodiments, the forward primer of a primer pair hybridises to at least 3, 4, 5, 6, 7 and preferably all of the following target sequences:
i. positions c1169825-1169806 of nucleotide sequence accession number NC_032093.1 (Candida albicans)
ii. positions c393798-393779 of nucleotide sequence accession number NC_005968.1 (Candida glabrata)
iii. positions c405996-405978 of nucleotide sequence accession number NW 003020040.1 (Candida tropicalis)
iv. positions c1218036-1218017 of nucleotide sequence accession number NC_012864.1 (Candida dubliniensis)
v. positions c909809-909790 of nucleotide sequence accession number NW 001809800.1 (Candida guilliermondii)
vi. positions c24006-23987 of nucleotide sequence accession number HE605203.1 (Candida parapsilosis)
vii. positions 61666-61685 of nucleotide sequence accession number JQFK01000016.1 (Candida krusei)
viii. positions c32324-32305 of nucleotide sequence accession number NW_017263971.1 (Candida auris).

According to some embodiments, the reverse primer of a primer pair hybridises to at least 3, 4, 5, 6, 7 and preferably all of the following target sequences:
i. positions 1169707-1169729 of nucleotide sequence accession number NC_032093.1 (Candida albicans)
ii. positions 393680-393702 of nucleotide sequence accession number NC_005968.1 (Candida glabrata)
iii. positions 405878-405900 of nucleotide sequence accession number NW_003020040.1 (Candida tropicalis)
iv. positions 1217918-1217940 of nucleotide sequence accession number NC_012864.1 (Candida dubliniensis)
v. positions 909691-909713 of nucleotide sequence accession number NW_001809800.1 (Candida guilliermondii)
vi. positions 23888-23910 of nucleotide sequence accession number HE605203.1 (Candida parapsilosis)
vii. positions c61784-61762 of nucleotide sequence accession number JQFK01000016.1 (Candida krusei)
viii. positions 32206-32228 of nucleotide sequence accession number NW_017263971.1 (Candida auris).

Thus, primer pairs may be generated from these particular target regions to permit pan-Candida detection. This can be achieved with a single primer pair in some embodiments.

In specific embodiments, the forward and reverse primer hybridizing specifically to the ILV3 gene of at least 3, 4, 5, 6, 7 and preferably all of the Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
   comprises, consists essentially of or consists of the nucleotide sequence of SEQ ID NO: 1 and SEQ ID NO: 2 respectively. This is a pan-Candida primer pair.

The invention also provides a forward and reverse primer hybridizing specifically to the ILV3 gene of the following Aspergillus species
i. Aspergillus fumigatus
ii. Aspergillus niger
iii. Aspergillus flavus

By “hybridising specifically”, or equivalent language, is meant that the primers hybridise to ILV3 from these 3 species but do not hybridise (or cross-react) with the ILV3 gene from non-Aspergillus species. Thus, an amplification product will only be generated if an Aspergillus species (from those 3 species) is present in the sample.

According to some embodiments, the forward primer of a primer pair hybridises to at least 2, and preferably all 3, of the following target sequences:
i. positions 3721583-3721597 of nucleotide sequence accession number NC_007195.1 (Aspergillus fumigatus)
ii. positions c541018-541004 of nucleotide sequence accession number NT_166533.1 (Aspergillus niger)
iii. positions c382612-382598 of nucleotide sequence accession number NW_002477240.1 (Aspergillus flavus)
or wherein the forward primer hybridises to at least 2, and preferably all 3, of the following target sequences:
i. positions 3721797-3721816 of nucleotide sequence accession number NC_007195.1 (Aspergillus fumigatus)
ii. positions c540804-540785 of nucleotide sequence accession number NT_166533.1 (Aspergillus niger)
iii. positions c382398-382379 of nucleotide sequence accession number NW_002477240.1 (Aspergillus flavus)

According to some embodiments, the reverse primer of a primer pair hybridises to at least 2, and preferably all 3, of the following target sequences:
i. positions c3721887-3721872 of nucleotide sequence accession number NC_007195.1 (Aspergillus fumigatus)
ii. positions 540714-540729 of nucleotide sequence accession number NT_166533.1 (Aspergillus niger)
iii. positions 382308-382323 of nucleotide sequence accession number NW_002477240.1 (Aspergillus flavus)
or wherein the reverse primer hybridises to at least 2, and preferably all 3, of the following target sequences:
i. positions c3721875-3721894 of nucleotide sequence accession number NC_007195.1 (Aspergillus fumigatus)
ii. positions 540707-540726 of nucleotide sequence accession number NT_166533.1 (Aspergillus niger)
iii. positions 382301-382320 of nucleotide sequence accession number NW_002477240.1 (Aspergillus flavus)

Thus, primer pairs may be generated from these particular target regions to permit pan-Aspergillus detection. This can be achieved with a single primer pair in some embodiments.

In specific embodiments the forward and reverse primer hybridizing specifically to the ILV3 gene of the following Aspergillus species
i. Aspergillus fumigatus
ii. Aspergillus niger
iii. Aspergillus flavus
   comprises, consists essentially of or consists of the nucleotide sequence of SEQ ID NO: 70 and 71 or SEQ ID NO: 73 and 74 respectively. Thus, SEQ ID NO: 70 and 71 form a preferred primer pair. SEQ ID NO: 73 and 74 form a second primer pair. These are pan-Aspergillus primer pairs.

The invention also provides a forward and reverse primer hybridizing specifically to the ILV3 gene of Candida albicans.

By “hybridising specifically”, or equivalent language, is meant that the primers hybridise to ILV3 from this species but do not hybridise (or cross-react) with the ILV3 gene from non-Candida albicans species. Thus, an amplification product will only be generated if Candida albicans is present in the sample and will not be generated if one of the other 7 Candida species is present in the sample (or if a non-Candida species is present).

According to some embodiments, the forward primer of a primer pair hybridizing specifically to the ILV3 gene of Candida albicans hybridises to one of the following target sequences from nucleotide sequence accession number NC_032093.1:
i. positions c1170234-1170217
ii. positions c1171213-1171192

According to some embodiments, the reverse primer of a primer pair hybridizing specifically to the ILV3 gene of Candida albicans hybridises to one of the following target sequences from nucleotide sequence accession number NC_032093.1:
i. positions 1170119-1170140
ii. positions 1171120-1171138

In specific embodiments the forward and reverse primer hybridizing specifically to the ILV3 gene of Candida albicans comprises, consists essentially of or consists of the nucleotide sequence of SEQ ID NO: 4 and 5 or 6 and 7 respectively. Thus, SEQ ID NO: 4 and 5 form a first primer pair. SEQ ID NO: 6 and 7 form a second primer pair.

The invention also provides a forward and reverse primer hybridizing specifically to the ILV3 gene of Candida dubliniensis.

By “hybridising specifically”, or equivalent language, is meant that the primers hybridise to ILV3 from this species but do not hybridise (or cross-react) with the ILV3 gene from non-Candida dubliniensis species. Thus, an amplification product will only be generated if Candida dubliniensis is present in the sample and will not be generated if one of the other 7 Candida species is present in the sample (or if a non-Candida species is present).

According to some embodiments, the forward primer of a primer pair hybridizing specifically to the ILV3 gene of Candida dubliniensis hybridises to one of the following target sequences from nucleotide sequence accession number NC_012864.1:
i. positions c1219181-1219161
ii. positions c1218505-1218486
iii. positions c1218553-1218534

According to some embodiments, the reverse primer of a primer pair hybridizing specifically to the ILV3 gene of Candida dubliniensis hybridises to one of the following target sequences from nucleotide sequence accession number NC_012864.1:
i. positions 1219103-1219124
ii. positions 1218409-1218429
iii. positions 1218419-1218440

In specific embodiments the forward and reverse primer hybridizing specifically to the ILV3 gene of Candida dubliniensis comprises, consists essentially of or consists of the nucleotide sequence of SEQ ID NO: 8 and 9, SEQ ID NO: 10 and 11 or SEQ ID NO: 12 and 13 respectively. Thus, SEQ ID NO: 8 and 9 form a first primer pair. SEQ ID NO: 10 and 11 form a second primer pair and so on.

The invention also provides a forward and reverse primer hybridizing specifically to the ILV3 gene of Candida tropicalis.

By “hybridising specifically”, or equivalent language, is meant that the primers hybridise to ILV3 from this species but do not hybridise (or cross-react) with the ILV3 gene from non-Candida tropicalis species. Thus, an amplification product will only be generated if Candida tropicalis is present in the sample and will not be generated if one of the other 7 Candida species is present in the sample (or if a non-Candida species is present).

According to some embodiments, the forward primer of a primer pair hybridizing specifically to the ILV3 gene of Candida tropicalis hybridises to one of the following target sequences from nucleotide sequence accession number NW_003020040.1:
i. positions c406146-406127
ii. positions c406142-406120

According to some embodiments, the reverse primer of a primer pair hybridizing specifically to the ILV3 gene of Candida tropicalis hybridises to one of the following target sequences from nucleotide sequence accession number NW_003020040.1:
i. positions 406047-406068
ii. positions 406048-406069

In specific embodiments the forward and reverse primer hybridizing specifically to the ILV3 gene of Candida tropicalis comprises, consists essentially of or consists of the nucleotide sequence of SEQ ID NO: 14 and 15 or SEQ ID NO: 16 and 17 respectively. Thus, SEQ ID NO: 14 and 15 form a first primer pair. SEQ ID NO: 16 and 17 form a second primer pair.

The invention also provides a forward and reverse primer hybridizing specifically to the ILV3 gene of Candida parapsilosis.

By “hybridising specifically”, or equivalent language, is meant that the primers hybridise to ILV3 from this species but do not hybridise (or cross-react) with the ILV3 gene from non-Candida parapsilosis species. Thus, an amplification product will only be generated if Candida parapsilosis is present in the sample and will not be generated if one of the other 7 Candida species is present in the sample (or if a non-Candida species is present).

According to some embodiments, the forward primer of a primer pair hybridizing specifically to the ILV3 gene of Candida parapsilosis hybridises to one of the following target sequences from nucleotide sequence accession number HE605203.1:
i. positions c24951-24931
ii. positions c24881-24863

According to some embodiments, the reverse primer of a primer pair hybridizing specifically to the ILV3 gene of Candida parapsilosis hybridises to one of the following target sequences from nucleotide sequence accession number HE605203.1:
i. positions 24843-24865
ii. positions 24774-24795

In specific embodiments the forward and reverse primer hybridizing specifically to the ILV3 gene of Candida parapsilosis comprises, consists essentially of or consists of the nucleotide sequence of SEQ ID NO: 18 and 19 or SEQ ID NO: 20 and 21 respectively. Thus, SEQ ID NO: 18 and 19 form a first primer pair. SEQ ID NO: 20 and 21 form a second primer pair.

The invention also provides a forward and reverse primer hybridizing specifically to the ILV3 gene of Candida glabrata.

By “hybridising specifically”, or equivalent language, is meant that the primers hybridise to ILV3 from this species but do not hybridise (or cross-react) with the ILV3 gene from non-Candida glabrata species. Thus, an amplification product will only be generated if Candida glabrata is present in the sample and will not be generated if one of the other 7 Candida species is present in the sample (or if a non-Candida species is present).

According to some embodiments, the forward primer of a primer pair hybridizing specifically to the ILV3 gene of Candida glabrata hybridises to one of the following target sequences from nucleotide sequence accession number NC_005968.1:
i. positions c394191-394169
ii. positions c393535-393516
iii. positions c395076-395055
iv. positions c394884-394863

According to some embodiments, the reverse primer of a primer pair hybridizing specifically to the ILV3 gene of Candida glabrata hybridises to one of the following target sequences from nucleotide sequence accession number NC_005968.1:
i. positions 394080-394101
ii. positions 393445-393464
iii. positions 394987-395006
iv. positions 394783-394803

In specific embodiments the forward and reverse primer hybridizing specifically to the ILV3 gene of Candida glabrata comprises, consists essentially of or consists of the nucleotide sequence of SEQ ID NO: 22 and 23, SEQ ID NO: 24 and 25, SEQ ID NO: 26 and 27 or SEQ ID NO: 28 and 29 respectively. Thus, SEQ ID NO: 22 and 23 form a first primer pair. SEQ ID NO: 24 and 25 form a second primer pair and so on.

The invention also provides a forward and reverse primer hybridizing specifically to the ILV3 gene of Candida krusei.

By “hybridising specifically”, or equivalent language, is meant that the primers hybridise to ILV3 from this species but do not hybridise (or cross-react) with the ILV3 gene from non-Candida krusei species. Thus, an amplification product will only be generated if Candida krusei is present in the sample and will not be generated if one of the other 7 Candida species is present in the sample (or if a non-Candida species is present).

According to some embodiments, the forward primer of a primer pair hybridizing specifically to the ILV3 gene of Candida krusei hybridises to one of the following target sequences from nucleotide sequence accession number JQFK01000016.1:
i. positions 61723-61744
ii. positions 60940-60961
iii. positions 60420-60440
iv. positions 61778-61797
v. positions 61940-61961

According to some embodiments, the reverse primer of a primer pair hybridizing specifically to the ILV3 gene of Candida krusei hybridises to one of the following target sequences from nucleotide sequence accession number JQFK01000016.1:
i. positions c61817-61796
ii. positions c61030-61011
iii. positions c60535-60513
iv. positions c61923-61902
v. positions c62015-61996

In specific embodiments the forward and reverse primer hybridizing specifically to the ILV3 gene of Candida krusei comprises, consists essentially of or consists of the nucleotide sequence of SEQ ID NO: 30 and 31, SEQ ID NO: 32 and 33, SEQ ID NO: 34 and 35, SEQ ID NO: 36 and 37 or SEQ ID NO: 38 and 39 respectively. Thus, SEQ ID NO: 30 and 31 form a first primer pair. SEQ ID NO: 32 and 33 form a second primer pair and so on.

The invention also provides a forward and reverse primer hybridizing specifically to the ILV3 gene of Candida guilliermondii.

By “hybridising specifically”, or equivalent language, is meant that the primers hybridise to ILV3 from this species but do not hybridise (or cross-react) with the ILV3 gene from non-Candida guilliermondii species. Thus, an amplification product will only be generated if Candida guilliermondii is present in the sample and will not be generated if one of the other 7 Candida species is present in the sample (or if a non-Candida species is present).

According to some embodiments, the forward primer of a primer pair hybridizing specifically to the ILV3 gene of Candida guilliermondii hybridises to one of the following target sequences from nucleotide sequence accession number NW_001809800.1:
i. positions c909629-909608
ii. positions c911117-911098
iii. positions c911111-911090
iv. positions c910941-910920

According to some embodiments, the reverse primer of a primer pair hybridizing specifically to the ILV3 gene of Candida guilliermondii hybridises to one of the following target sequences from nucleotide sequence accession number NW_001809800.1:
i. positions 909529-909550
ii. positions 911002-911023
iii. positions 910994-911013
iv. positions 910799-910819

In specific embodiments the forward and reverse primer hybridizing specifically to the ILV3 gene of Candida guilliermondii comprises, consists essentially of or consists of the nucleotide sequence of SEQ ID NO: 40 and 41, SEQ ID NO: 42 and 43, SEQ ID NO: 44 and 45 or SEQ ID NO: 46 and 47 respectively. Thus, SEQ ID NO: 40 and 41 form a first primer pair. SEQ ID NO: 42 and 43 form a second primer pair and so on.

The invention also provides a forward and reverse primer hybridizing specifically to the ILV3 gene of Candida auris.

By “hybridising specifically”, or equivalent language, is meant that the primers hybridise to ILV3 from this species but do not hybridise (or cross-react) with the ILV3 gene from non-Candida auris species. Thus, an amplification product will only be generated if Candida auris is present in the sample and will not be generated if one of the other 7 Candida species is present in the sample (or if a non-Candida species is present).

According to some embodiments, the forward primer of a primer pair hybridizing specifically to the ILV3 gene of Candida auris hybridises to one of the following target sequences from nucleotide sequence accession number NW_017263971.1:
i. positions c32790-32768
ii. positions c32451-32430
iii. positions c32654-32633
iv. positions c33278-33259
v. positions c32603-32580
vi. positions c32399-32376
vii. positions c33648-33628
viii. positions c32240-32219
ix. positions c32930-32909
x. positions c33228-33206
xi. positions c32106-32085

According to some embodiments, the reverse primer of a primer pair hybridizing specifically to the ILV3 gene of Candida auris hybridises to one of the following target sequences from nucleotide sequence accession number NW_017263971.1:
i. positions 32682-32703
ii. positions 32368-32389
iii. positions 32567-32588
iv. positions 33202-33223
v. positions 32512-32533
vi. positions 32328-32349
vii. positions 33566-33587
viii. positions 32175-32195
ix. positions 32826-32848
x. positions 33130-33151
xi. positions 32011-32033

In specific embodiments the forward and reverse primer hybridizing specifically to the ILV3 gene of Candida auris comprises, consists essentially of or consists of the nucleotide sequence of SEQ ID NO: 48 and 49, SEQ ID NO: 50 and 51, SEQ ID NO: 52 and 53, SEQ ID NO: 54 and 55, SEQ ID NO: 56 and 57, SEQ ID NO: 58 and 59, SEQ ID NO: 60 and 61, SEQ ID NO: 62 and 63, SEQ ID NO: 64 and 65, SEQ ID NO: 66 and 67 or SEQ ID NO: 68 and 69 respectively. Thus, SEQ ID NO: 48 and 49 form a first primer pair. SEQ ID NO: 50 and 51 form a second primer pair and so on.

The invention also provides a forward and reverse primer hybridizing specifically to the ILV3 gene of Aspergillus fumigatus.

By “hybridising specifically”, or equivalent language, is meant that the primers hybridise to ILV3 from this species but do not hybridise (or cross-react) with the ILV3 gene from non-Aspergillus fumigatus species. Thus, an amplification product will only be generated if Aspergillus fumigatus is present in the sample and will not be generated if one of the other 2 Aspergillus species is present in the sample (or if a non-Aspergillus species is present).

According to some embodiments, the forward primer of a primer pair hybridizing specifically to the ILV3 gene of Aspergillus fumigatus hybridises to one of the following target sequences from nucleotide sequence accession number NC_007195.1:
i. positions 3721531-3721552
ii. positions 3721618-3721638
iii. positions 3721616-3721638
iv. positions 3721798-3721818

According to some embodiments, the reverse primer of a primer pair hybridizing specifically to the ILV3 gene of Aspergillus fumigatus hybridises to one of the following target sequences from nucleotide sequence accession number NC_007195.1:
i. positions c3721653-3721675
ii. positions c3721706-3721681
iii. positions c3721898-3721878

In specific embodiments the forward and reverse primer hybridizing specifically to the ILV3 gene of Aspergillus fumigatus comprises, consists essentially of or consists of the nucleotide sequence of SEQ ID NO: 76 and 77, SEQ ID NO: 79 and 80, SEQ ID NO: 82 and 80 or SEQ ID NO: 83 and 84 respectively. Thus, SEQ ID NO: 76 and 77 form a first primer pair. SEQ ID NO: 79 and 80 form a second primer pair and so on.

The invention also provides a forward and reverse primer hybridizing specifically to the ILV3 gene of Aspergillus niger.

By “hybridising specifically”, or equivalent language, is meant that the primers hybridise to ILV3 from this species but do not hybridise (or cross-react) with the ILV3 gene from non-Aspergillus niger species. Thus, an amplification product will only be generated if Aspergillus niger is present in the sample and will not be generated if one of the other 2 Aspergillus species is present in the sample (or if a non-Aspergillus species is present).

According to some embodiments, the forward primer of a primer pair hybridizing specifically to the ILV3 gene of Aspergillus niger hybridises to one of the following target sequences from nucleotide sequence accession number NT_166533.1:
i. positions c540219-540199

According to some embodiments, the reverse primer of a primer pair hybridizing specifically to the ILV3 gene of Aspergillus niger hybridises to one of the following target sequences from nucleotide sequence accession number NT_166533.1:
ii. positions 540084-540103

In specific embodiments the forward and reverse primer hybridizing specifically to the ILV3 gene of Aspergillus niger comprises, consists essentially of or consists of the nucleotide sequence of SEQ ID NO: 87 and 86.

The invention also provides a forward and reverse primer hybridizing specifically to the ILV3 gene of Aspergillus flavus.

By “hybridising specifically”, or equivalent language, is meant that the primers hybridise to ILV3 from this species but do not hybridise (or cross-react) with the ILV3 gene from non-Aspergillus flavus species. Thus, an amplification product will only be generated if Aspergillus flavus is present in the sample and will not be generated if one of the other Aspergillus species is present in the sample (or if a non-Aspergillus species is present)

According to some embodiments, the forward primer of a primer pair hybridizing specifically to the ILV3 gene of Aspergillus flavus hybridises to one of the following target sequences from nucleotide sequence accession number NW_002477240.1:
i. positions c382604-382586

According to some embodiments, the reverse primer of a primer pair hybridizing specifically to the ILV3 gene of Aspergillus flavus hybridises to one of the following target sequences from nucleotide sequence accession number NW_002477240.1:
i. positions 382519-382541
ii. positions 382520-382541

In specific embodiments the forward and reverse primer hybridizing specifically to the ILV3 gene of Aspergillus flavus comprises, consists essentially of or consists of the nucleotide sequence of SEQ ID NO: 90 and 89 or SEQ ID NO: 90 and 92 respectively. Thus, SEQ ID NO: 90 and 89 form a first primer pair. SEQ ID NO: 90 and 92 form a second primer pair.

The invention further provides a forward and reverse primer hybridizing specifically to the ILV3 gene of Cryptococcus neoformans.

By “hybridising specifically”, or equivalent language, is meant that the primers hybridise to ILV3 from this species but do not hybridise (or cross-react) with the ILV3 gene from non-Cryptococcus neoformans species. Thus, an amplification product will only be generated if Cryptococcus neoformans is present in the sample and will not be generated if a non-Cryptococcus neoformans species is present.

According to some embodiments, the forward primer of a primer pair hybridizing specifically to the ILV3 gene of Cryptococcus neoformans hybridises to one of the following target sequences from nucleotide sequence accession number NC_006693.1:
i. Positions 702696-702717
ii. Positions 702499-702520
iii. Positions 702736-702757
iv. Positions 702384-702403
v. Positions 701384-701406

According to some embodiments, the reverse primer of a primer pair hybridizing specifically to the ILV3 gene of Cryptococcus neoformans hybridises to one of the following target sequences from nucleotide sequence accession number NC_006693.1:
i. Positions c702796-702777
ii. Positions c702602-702582
iii. Positions c702850-702831
iv. Positions c702521-702500
v. Positions c701499-701479

In specific embodiments the forward and reverse primer hybridizing specifically to the ILV3 gene of Cryptococcus neoformans comprises, consists essentially of or consists of the nucleotide sequence of SEQ ID NO: 93 and 94, SEQ ID NO: 96 and 97, SEQ ID NO: 99 and 100, SEQ ID NO: 102 and 103 or SEQ ID NO: 105 and 106 respectively. Thus, SEQ ID NO: 93 and 94 form a first primer pair. SEQ ID NO: 95 and 97 form a second primer pair and so on.

The primer pairs, as explained herein, are preferably used in combination for example in multiplex reactions. Multiple primer pairs can be included in a single reaction mixture (a mastermix). Thus, in some embodiments, at least one primer in each primer pair is differentially labelled compared to the other primer pairs. This is one means by which amplification products can be distinguished. Examples of labelled primers that may be used in the present invention include AMPLIFLUOR primers and LUX primers. Thus primers may include modifications, labels and sequence extensions to incorporate the relevant detection technology. Such sequence modifications, labels and extensions are encompassed by the invention.

Many nucleic acid amplification protocols involve use of a probe. Variants of PCR permit detection in real time or at end-point using such probes. Examples include hydrolytic probes (e.g. TAQMAN probes) and hairpin probes (e.g. MOLECULAR BEACONS). Probes may also be attached to primers in some embodiments (e.g. SCORPION probes). Thus probes of the invention may include modifications, labels and sequence extensions to incorporate the relevant detection technology. Such sequence modifications, labels and extensions are encompassed by the invention. Preferably, the probes of the invention also target the ILV3 gene in genus or species specific fashion to complement the action of the primers. In relation to the probes of the invention “hybridising specifically” is defined in analogous fashion to the definitions provided for the corresponding primers. Again, Table B identifies preferred combinations of probes of the invention with primer pairs of the invention, including when defined by reference to their target sequence.

The invention therefore provides at least one probe for detecting a yeast/fungus infection in a sample comprising a probe that hybridizes specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris

According to some embodiments the at least one probe hybridises to at least 3, 4, 5, 6, 7 and preferably all of the following target sequences:
i. positions 1169779-1169804 of nucleotide sequence accession number NC_032093.1 (Candida albicans)
ii. positions 393752-393777 of nucleotide sequence accession number NC_005968.1 (Candida glabrata)
iii. positions 405950-405975 of nucleotide sequence accession number NW_003020040.1 (Candida tropicalis)
iv. positions 1217990-1218015 of nucleotide sequence accession number NC_012864.1 (Candida dubliniensis)
v. positions 909763-909788 of nucleotide sequence accession number NW_001809800.1 (Candida guilliermondii)
vi. positions 23960-23985 of nucleotide sequence accession number HE605203.1 (Candida parapsilosis)
vii. positions c61712-61687 of nucleotide sequence accession number JQFK01000016.1 (Candida krusei)
viii. positions 32278-32303 of nucleotide sequence accession number NW_017263971.1 (Candida auris).

In specific embodiments, the probe that hybridizes specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
   comprises, consists essentially of or consists of the nucleotide sequence of SEQ ID NO: 3.

The invention also provides at least one probe for detecting a yeast/fungus infection in a sample comprising a probe that hybridizes specifically to the ILV3 gene of the following Aspergillus species
i. Aspergillus fumigatus
ii. Aspergillus niger
iii. Aspergillus flavus

According to some embodiments at least one probe hybridises to at least 2, and preferably all 3, of the following target sequences:
i. positions 3721790-3721814 of nucleotide sequence accession number NC_007195.1 (Aspergillus fumigatus)
ii. positions c540811-540788 of nucleotide sequence accession number NT_166533.1 (Aspergillus niger)
iii. positions c382405-382381 of nucleotide sequence accession number NW_002477240.1 (Aspergillus flavus)
   or the at least one probe hybridises to at least 2, and preferably all 3, of the following target sequences:
i. positions 3721848-3721870 of nucleotide sequence accession number NC_007195.1 (Aspergillus fumigatus)
ii. positions c540753-540731 of nucleotide sequence accession number NT_166533.1 (Aspergillus niger)
iii. positions c382347-382325 of nucleotide sequence accession number NW_002477240.1 (Aspergillus flavus).

In specific embodiments the probe that hybridizes specifically to the ILV3 gene of the following Aspergillus species
i. Aspergillus fumigatus
ii. Aspergillus niger
iii. Aspergillus flavus
   comprises, consists essentially of or consists of the nucleotide sequence of SEQ ID NO: 72 or 75.

The invention also provides at least one probe for detecting a yeast/fungus infection in a sample comprising a probe that hybridizes specifically to the ILV3 gene of Candida albicans.

According to some embodiments, the probe hybridizing specifically to the ILV3 gene of Candida albicans hybridises to one of the following target sequences from nucleotide sequence accession number NC_032093.1:
i. positions c1170174-1170151
ii. positions 1171165-1171192

In specific embodiments the probe hybridizing specifically to the ILV3 gene of Candida albicans comprises, consists essentially of or consists of the nucleotide sequence of SEQ ID NO: 116 or 117.

The invention therefore provides at least one probe for detecting a yeast/fungus infection in a sample comprising a probe that hybridizes specifically to the ILV3 gene of Candida dubliniensis.

According to some embodiments, the probe hybridizing specifically to the ILV3 gene of Candida dubliniensis hybridises to one of the following target sequences from nucleotide sequence accession number NC_012864.1:
i. positions 1219148-1219171
ii. positions c1218439-1218416
iii. positions c1218505-1218480

In specific embodiments the probe hybridizing specifically to the ILV3 gene of Candida dubliniensis comprises, consists essentially of or consists of the nucleotide sequence of SEQ ID NO: 118, 119 or 120.

The invention therefore provides at least one probe for detecting a yeast/fungus infection in a sample comprising a probe that hybridizes specifically to the ILV3 gene of Candida tropicalis.

According to some embodiments, the probe hybridizing specifically to the ILV3 gene of Candida tropicalis hybridises to the following target sequence from nucleotide sequence accession number NW_003020040.1:
i. positions c406111-406082

In specific embodiments the probe hybridizing specifically to the ILV3 gene of Candida tropicalis comprises, consists essentially of or consists of the nucleotide sequence of SEQ ID NO: 121.

The invention therefore provides at least one probe for detecting a yeast/fungus infection in a sample comprising a probe that hybridizes specifically to the ILV3 gene of Candida parapsilosis.

According to some embodiments, the probe hybridizing specifically to the ILV3 gene of Candida parapsilosis hybridises to one of the following target sequences from nucleotide sequence accession number HE605203.1:
i. positions c24907-24883
ii. positions c24862-24839

In specific embodiments the probe hybridizing specifically to the ILV3 gene of Candida parapsilosis comprises, consists essentially of or consists of the nucleotide sequence of SEQ ID NO: 122 or 123.

The invention therefore provides at least one probe for detecting a yeast/fungus infection in a sample comprising a probe that hybridizes specifically to the ILV3 gene of Candida glabrata.

According to some embodiments, the probe hybridizing specifically to the ILV3 gene of Candida glabrata hybridises to one of the following target sequences from nucleotide sequence accession number NC_005968.1:
i. positions c394151-394128
ii. positions c393478-393453
iii. positions c395030-395007
iv. positions c394836-394807

In specific embodiments the probe hybridizing specifically to the ILV3 gene of Candida glabrata comprises, consists essentially of or consists of the nucleotide sequence of SEQ ID NO: 124, 125, 126 or 127.

The invention therefore provides at least one probe for detecting a yeast/fungus infection in a sample comprising a probe that hybridizes specifically to the ILV3 gene of Candida krusei.

According to some embodiments, the probe hybridizing specifically to the ILV3 gene of Candida krusei hybridises to one of the following target sequences from nucleotide sequence accession number JQFK01000016.1:
i. positions 61770-61793
ii. positions c60990-60967
iii. positions 60462-60485
iv. positions 61815-61838
v. positions c61970-61945

In specific embodiments the probe hybridizing specifically to the ILV3 gene of Candida krusei comprises, consists essentially of or consists of the nucleotide sequence of SEQ ID NO: 128, 129, 130, 131 or 132.

The invention therefore provides at least one probe for detecting a yeast/fungus infection in a sample comprising a probe that hybridizes specifically to the ILV3 gene of Candida guilliermondii.

According to some embodiments, the probe hybridizing specifically to the ILV3 gene of Candida guilliermondii hybridises to one of the following target sequences from nucleotide sequence accession number NW_001809800.1:
i. positions c909577-909552
ii. positions c911088-911065
iii. positions 910878-910904

In specific embodiments the probe hybridizing specifically to the ILV3 gene of Candida guilliermondii comprises, consists essentially of or consists of the nucleotide sequence of SEQ ID NO: 133, 134 or 135.

The invention therefore provides at least one probe for detecting a yeast/fungus infection in a sample comprising a probe that hybridizes specifically to the ILV3 gene of Candida auris.

According to some embodiments, the probe hybridizing specifically to the ILV3 gene of Candida auris hybridises to one of the following target sequences from nucleotide sequence accession number NW_017263971.1:
i. positions c32744-32721
ii. positions c32421-32398
iii. positions c32629-32605
iv. positions 33233-33257
v. positions 32542-32563
vi. positions 32350-32373
vii. positions c33627-33606
viii. positions c32218-32196
ix. positions c32890-32864
x. positions c33259-33236
xi. positions c32061-32038

In specific embodiments the probe hybridizing specifically to the ILV3 gene of Candida auris comprises, consists essentially of or consists of a nucleotide sequence selected from SEQ ID NO: 136-146.

The invention therefore provides at least one probe for detecting a yeast/fungus infection in a sample comprising a probe that hybridizes specifically to the ILV3 gene of Aspergillus fumigatus.

According to some embodiments the probe hybridizing specifically to the ILV3 gene of Aspergillus fumigatus hybridises to one of the following target sequences from nucleotide sequence accession number NC_007195.1:
i. positions c3721651-3721628
ii. positions 3721658-3721681
iii. positions 3721848-3721870

In specific embodiments the probe that hybridizes specifically to the ILV3 gene of Aspergillus fumigatus comprises, consists essentially of or consists of the nucleotide sequence of SEQ ID NO: 78, 81 or 85.

The invention also provides at least one probe for detecting a yeast/fungus infection in a sample comprising a probe that hybridizes specifically to the ILV3 gene of Aspergillus niger

According to some embodiments the probe hybridizing specifically to the ILV3 gene of Aspergillus niger hybridises to one of the following target sequences from nucleotide sequence accession number NT_166533.1:
i. positions c540161-540138.

In specific embodiments the probe that hybridizes specifically to the ILV3 gene of Aspergillus niger comprises, consists essentially of or consists of the nucleotide sequence of SEQ ID NO: 88.

The invention also provides at least one probe for detecting a yeast/fungus infection in a sample comprising a probe that hybridizes specifically to the ILV3 gene of Aspergillus flavus.

According to some embodiments the probe hybridizing specifically to the ILV3 gene of Aspergillus flavus hybridises to one of the following target sequences from nucleotide sequence accession number NW_002477240.1:
i. positions c382572-382549

In specific embodiments the probe that hybridizes specifically to the ILV3 gene of Aspergillus flavus comprises, consists essentially of or consists of the nucleotide sequence of SEQ ID NO: 91.

The invention also provides at least one probe for detecting a yeast/fungus infection in a sample comprising a probe that hybridizes specifically to the ILV3 gene of Cryptococcus neoformans.

According to some embodiments the probe hybridizing specifically to the ILV3 gene of Cryptococcus neoformans hybridises to one of the following target sequences from nucleotide sequence accession number NC_006693.1:
i. Positions 702724-702747
ii. Positions 702526-702549
iii. Positions 702805-702829
iv. Positions 702423-702446
v. Positions 701435-701458

In specific embodiments the probe that hybridizes specifically to the ILV3 gene of Cryptococcus neoformans comprises, consists essentially of or consists of the nucleotide sequence of SEQ ID NO: 95, 98, 101, 104 or 107.

The probes, as explained herein, are preferably used in combination for example in multiplex reactions. The invention provides sets of probes comprising at least two probes of the invention and which are intended to be used together (e.g. in a multiplex reaction and/or mastermix). Thus, in some embodiments, each probe is differentially labelled compared to the other probes (that are used in the amplification). As already discussed, many nucleic acid amplification protocols involve use of a probe. Examples include hydrolytic probes (e.g. TAQMAN probes) and hairpin probes (e.g. MOLECULAR BEACONS). Probes may also be attached to primers in some embodiments (e.g. SCORPION probes). Such probes may be differentially labelled as would be readily understood by one skilled in the art. This may involve inclusion of different fluorophores and/or different quenchers.

The primers and probes of the invention (which may be referred to as “detection components”) are advantageously combined together to facilitate nucleic acid amplification based fungal detection, and characterisation in some embodiments. Preferred combinations of primer pairs and probes are set forth in Table B. Thus, the invention also provides a kit for detecting a yeast/fungus infection in a sample comprising at least one primer pair of the invention and/or at least one probe of the invention.

Kits containing pan-Candida and pan-Aspergillus detection components may be combined. They may be further combined with detection components to detect Cryptococcus neoformans. Thus, the kit may comprise combinations of primer pairs permitting detection of Candida, Aspergillus and Cryptococcus neoformans. The primer pairs may be provided in the form of a mastermix combination (i.e. a single master mix containing the primer pairs at suitable concentrations). Such kits may further comprise the relevant probes permitting detection of Candida, Aspergillus and Cryptococcus neoformans. The probes may also be included in the mastermix combination (again at a suitable concentration). One specific kit useful according to the invention comprises primers that comprise, consist essentially of or consist of the nucleotide sequences of SEQ ID NOs: 1 and 2 (for pan-Candida detection), SEQ ID NOs: 70 and 71 (for pan-Aspergillus detection) and SEQ ID NOs: 93 and 94 (for Cryptococcus neoformans detection). Such a kit may further comprise the probes of SEQ ID NOs: 3, 72 and 95 respectively.

Other kits of the invention, which may be combined with the kits described above, are useful for identifying the species responsible for a Candida infection in a sample. They contain appropriate Candida species specific primers of the invention. The primer pairs may be provided in the form of a mastermix combination (i.e. a single master mix containing the primer pairs at suitable concentrations). One specific kit useful according to the invention comprises primers that comprise, consist essentially of or consist of the nucleotide sequences of SEQ ID Nos 48 and 49, 18 and 19, 24 and 25, 40 and 41, 6 and 7, 8 and 9, 16 and 17 and 38 and 39 respectively.

Other kits of the invention, which may be combined with the kits described above, are useful for identifying the species responsible for an Aspergillus infection in a sample. They contain appropriate Aspergillus species specific primers of the invention. The primer pairs may be provided in the form of a mastermix combination (i.e. a single master mix containing the primer pairs at suitable concentrations). One specific kit useful according to the invention comprises primers that comprise, consist essentially of or consist of the nucleotide sequences of SEQ ID NOs 80 and 82, 86 and 87, 90 and 92.

The kits of the invention may contain various additional components. For example, they may contain reagents needed for amplification. They may contain one or more of a polymerase, dNTPs, MgCl₂, buffer etc. In some embodiments the kits may include DNA extraction reagents. More specifically, the kits may include reagents for extracting DNA from a blood sample. The kits may incorporate a suitable carrier in which the amplification reactions take place. Advantageously, such a carrier may comprise a multi-well plate, such as a 48 or 96 well plate for example. Such a carrier allows the detection methods to be carried out in relatively small volumes—thus facilitating scale up and minimising the sample volume required.

The kits will typically incorporate suitable instructions. These instructions permit the methods of the invention to be carried out reliably using the kits of the invention.

While particular primers and probes have been extensively described and are usefully applied in the methods of the invention, other primers and probes may be designed and applied to target ILV3. Accordingly, the invention provides a general method of detecting a fungal/yeast infection in a sample, comprising:

a. performing a nucleic acid amplification reaction to amplify the ILV3 gene of fungi/yeast
b. detecting the amplification product to determine whether the sample contains a fungal/yeast infection.

According to all aspects of the invention ILV3 may be used to identify any fungus/yeast of interest. However, by “amplify the ILV3 gene” it is not intended that the entire ILV3 gene must be amplified. As the skilled person would be readily aware, only a portion of the ILV3 gene need be amplified to indicate the presence of the ILV3 gene. The minimum size of amplification product is typically governed by the primer length (and probe if included). Typical amplification products may be between 50 and 500 nucleotides in length, such as between 50 and 250 nucleotides. “Infection” simply refers to the presence of the fungus/yeast in a sample which ordinarily would not contain such fungus or yeast. Thus, the methods of the invention are also sensitive as well as specific to enable even low levels of fungal cells to be determined in the sample. A “sample” in the context of the present invention is thus defined to include any sample in which it is desirable to test for the presence of a fungus (e.g. a yeast) carrying the ILV3 gene. The sample may not, a priori, be known to contain a fungus. The sample may be obtained from a human subject. The sample may, therefore, contain human genetic material (in particular human DNA). Thus the sample may comprise, consist essentially of or consist of a clinical sample, such as a blood sample. By blood sample is meant any sample comprising blood or a derivative thereof. Thus, serum and plasma are included together with blood broth (i.e. blood added to a culture medium). The methods of the invention are particularly applicable to the rapid detection and identification of the source of a fungal infection. Thus, the sample may comprise a blood culture sample from a patient suspected of suffering from, or being screened for, a bloodstream infection. The sample may be any suitable volume such as 1 to 10 ml, preferably a 1 ml blood culture sample.

Alternatively the sample may be or comprise an in vitro assay system for example. Samples may comprise, consist essentially of or consist of beverage or food samples or preparations thereof, or pharmaceutical or cosmetic products such as personal care products including shampoos, conditioners, moisturisers etc., all of which are tested for microbial contamination as a matter of routine. The sample may comprise, consist essentially of or consist of tissue or cells and may comprise, consist essentially of or consist of a sputum or a blood sample or a platelet sample for example. In addition, the methods and kits of the invention may be used to monitor contamination of surfaces, such as for example in locations where food is being prepared. The contamination may be from any relevant fungal source. Furthermore, the invention is also useful in monitoring environmental conditions such as water supplies, wastewater, marine environments etc.

While the invention is applicable to potentially any fungus or yeast, there are particular fungi that are of importance to clinical diagnoses. Thus, the invention has been developed to target fungal genera and species that cause blood borne infections at relatively high frequency. Thus, the invention may focus on detection and optionally discrimination of Candida species. The invention may permit detection of at least 1, 2, 3, 4, 5, 6, 7 or all 8 of the following species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris

This may be via pan-Candida targeting or by species-specific targeting of the ILV3 gene as explained in further detail herein. The invention may additionally, or alternatively, focus on detection and optionally discrimination of Aspergillus species. The invention may permit detection of at least 1, 2 or all 3 of the following species:
i. Aspergillus fumigatus
ii. Aspergillus niger
iii. Aspergillus flavus

This may be via pan-Aspergillus targeting or by species-specific targeting of the ILV3 gene as explained in further detail herein. The invention may additionally, or alternatively, focus on detection of Cryptococcus neoformans.

The invention thus provides a method of detecting a fungal/yeast infection in a sample, comprising:
a. performing a nucleic acid amplification reaction comprising the following components:
   i. a forward and reverse primer hybridizing specifically to the ILV3 gene of Candida species, optionally together with a probe that hybridizes between the primer binding sites specifically to the ILV3 gene of Candida species; and
   ii. a forward and reverse primer hybridizing specifically to the ILV3 gene of Aspergillus species, optionally together with a probe that hybridizes between the primer binding sites specifically to the ILV3 gene of Aspergillus species; and/or
   iii. a forward and reverse primer hybridizing specifically to the ILV3 gene of Cryptococcus neoformans, optionally together with a probe that hybridizes between the primer binding sites specifically to the ILV3 gene of Cryptococcus neoformans
b. detecting the amplification products to determine whether the sample contains a fungal/yeast infection.

The methods of the invention may involve a nucleic acid amplification reaction that is capable of amplifying, in specific fashion, the ILV3 gene of at least 1, 2, 3, 4, 5, 6, 7, 8, 9, 10, 11 or all 12 of the following species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
ix. Aspergillus fumigatus
x. Aspergillus niger
xi. Aspergillus flavus
xii. Cryptococcus neoformans.

The amplification used in the methods of the invention may involve use of a forward and reverse primer hybridizing specifically to the ILV3 gene of Candida species. Additionally or alternatively, the methods may involve use of a probe that hybridizes specifically to the ILV3 gene of Candida species. In some embodiments, a common forward and reverse primer and/or common probe hybridises to the ILV3 gene of at least 2, 3, 4, 5, 6, 7 and preferably all, of the following Candida species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris

Thus, in some embodiments, pan-Candida amplification is utilised. Suitable primers and probes of the invention for pan-Candida amplification are described herein, including specific targeting regions within ILV3, and all such primers and probes may be utilised. In one embodiment, the methods of the invention comprise use of a forward primer comprising the sequence of SEQ ID NO: 1, a reverse primer comprising the sequence of SEQ ID NO: 2 and/or a probe comprising the sequence of SEQ ID NO: 3.

In other embodiments, including methods for discriminating the source of the infection, Candida species specific amplification is adopted. In such embodiments, a separate forward and reverse primer and/or probe hybridises to the ILV3 gene of each of at least 2, 3, 4, 5, 6, 7 and preferably all, of the following Candida species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris

Thus, there is a separate primer pair and/or probe for each Candida species to be detected. Suitable primers and probes of the invention for species specific Candida amplification are described herein, including specific targeting regions within ILV3, and all such primers and probes may be utilised.

The amplification used in the methods of the invention may additionally or alternatively involve use of a forward and reverse primer hybridizing specifically to the ILV3 gene of Aspergillus species. Additionally or alternatively, the methods may involve use of a probe that hybridizes specifically to the ILV3 gene of Aspergillus species. In some embodiments, a common forward and reverse primer and/or common probe hybridises to the ILV3 gene of at least 2, and preferably all 3, of the following Aspergillus species:
i. Aspergillus fumigatus
ii. Aspergillus niger
iii. Aspergillus flavus

Thus, in some embodiments, pan-Aspergillus amplification is utilised. Suitable primers and probes of the invention for pan-Aspergillus amplification are described herein, including specific targeting regions within ILV3, and all such primers and probes may be utilised. In one embodiment, the methods of the invention comprise use of a forward primer comprising the sequence of SEQ ID NO: 70 or 73, a reverse primer comprising the sequence of SEQ ID NO: 71 or 74 and/or a probe comprising the sequence of SEQ ID NO: 72 or 75.

In other embodiments, including methods for discriminating the source of the infection, Aspergillus species specific amplification is adopted. In such embodiments, a separate forward and reverse primer and/or probe hybridises to the ILV3 gene of each of at least 2, and preferably all 3, of the following Aspergillus species:
i. Aspergillus fumigatus
ii. Aspergillus niger
iii. Aspergillus flavus.

Thus, there is a separate primer pair and/or probe for each Aspergillus species to be detected. Suitable primers and probes of the invention for species specific Aspergillus amplification are described herein, including specific targeting regions within ILV3, and all such primers and probes may be utilised.

The amplification used in the methods of the invention may additionally or alternatively involve use of a forward and reverse primer hybridizing specifically to the ILV3 gene of Cryptococcus neoformans. Additionally or alternatively, the methods may involve use of a probe that hybridizes specifically to the ILV3 gene of Cryptococcus neoformans. Suitable primers and probes of the invention for Cryptococcus neoformans amplification are described herein, including specific targeting regions within ILV3, and all such primers and probes may be utilised.

According to the methods of the invention, amplification products can simply be detected to indicate a fungal species is present in the sample. Amplification products can be detected via any known means as would be readily appreciated by one skilled in the art. In some embodiments, however, discrimination of amplification products is used in order to identify the genus and/or species of the fungus present in the sample. Thus, methods of the invention may involve detecting and identifying a fungal/yeast infection in a sample. They may comprise performing the nucleic acid amplification and detecting and distinguishing the amplification products to identify the fungal/yeast infection.

The invention therefore provides a method of detecting and identifying a fungal/yeast infection in a sample, comprising:
a. performing a nucleic acid amplification reaction comprising the following components:
   i. a forward and reverse primer hybridizing specifically to the ILV3 gene of Candida species, optionally together with a probe that hybridizes between the primer binding sites specifically to the ILV3 gene of Candida species; and
   ii. a forward and reverse primer hybridizing specifically to the ILV3 gene of Aspergillus species, optionally together with a probe that hybridizes between the primer binding sites specifically to the ILV3 gene of Aspergillus species; and/or
   iii. a forward and reverse primer hybridizing specifically to the ILV3 gene of Cryptococcus neoformans, optionally together with a probe that hybridizes between the primer binding sites specifically to the ILV3 gene of Cryptococcus neoformans
b. detecting and distinguishing the amplification products to identify the fungal/yeast infection.

The methods of the invention that permit the identity of the fungus in the sample to be identified are particularly useful to direct treatment in a manner that is specific for the infection at hand. They are thus useful separately from general fungal detection methods.

Thus, the invention also relates to methods of identifying the species responsible for a Candida infection in a sample, comprising:
a. performing nucleic acid amplification reactions using at least three, 4, 5, 6, 7 or all of the following sets of components:
   i. a forward and reverse primer hybridizing specifically to the ILV3 gene of Candida albicans
   ii. a forward and reverse primer hybridizing specifically to the ILV3 gene of Candida dubliniensis
   iii. a forward and reverse primer hybridizing specifically to the ILV3 gene of Candida tropicalis
   iv. a forward and reverse primer hybridizing specifically to the ILV3 gene of Candida parapsilosis
   v. a forward and reverse primer hybridizing specifically to the ILV3 gene of Candida glabrata
   vi. a forward and reverse primer hybridizing specifically to the ILV3 gene of Candida krusei
   vii. a forward and reverse primer hybridizing specifically to the ILV3 gene of Candida guilliermondii
   viii. a forward and reverse primer hybridizing specifically to the ILV3 gene of Candida auris
b. detecting and distinguishing the amplification products to identify the species responsible for the Candida infection.

These methods may employ any of the suitable primers (and probes) of the invention that are described herein in detail.

Similarly, the invention also provides a method of identifying the species responsible for an Aspergillus infection in a sample, comprising:
a. performing nucleic acid amplification reactions using at least two or all three of the following sets of components:
   i. a forward and reverse primer hybridizing specifically to the ILV3 gene of Aspergillus fumigatus
   ii. a forward and reverse primer hybridizing specifically to the ILV3 gene of Aspergillus niger
   iii. a forward and reverse primer hybridizing specifically to the ILV3 gene of Aspergillus flavus
b. detecting and distinguishing the amplification products to identify the species responsible for the Aspergillus infection.

These methods may employ any of the suitable primers (and probes) of the invention that are described herein in detail. These methods may be employed in parallel with Candida identification methods in some embodiments.

The invention provides methods that can be considered a general screen to determine whether a fungal infection is present. The invention also provides more specific methods that permit the species responsible for the infection to be identified. Such methods may advantageously be combined. Accordingly, the invention further provides a method of detecting and identifying a yeast/fungal infection in a sample comprising:
a. Performing a method of the invention in order to determine whether Candida, Aspergillus and/or Cryptococcus neoformans is present in the sample
b. In the event that a species of Candida or Aspergillus is present in the sample performing a method of the invention in order to determine which species is present
to thereby detect and identify the yeast/fungal infection in the sample.

In step a, the detection of Cryptococcus neoformans may remove the requirement to perform step b. Alternatively, the outcome of step a may simply be that there is a fungus present in the sample. In such methods, step b may additionally comprise performing a method of the invention to determine whether Cryptococcus neoformans specifically is present in the sample.

As discussed herein, the methods of the invention generally involve nucleic acid amplification of the ILV3 gene. Any form of nucleic acid amplification can be used, although polymerase chain reaction (PCR) is preferred. Such methods may employ any suitable form of detection technology. Real-time monitoring of amplification may be used in some embodiments. In other embodiments, an end-point detection method may be employed. In some embodiments, the nucleic acid amplification is performed as a multiplex nucleic acid amplification reaction. In other embodiments, each nucleic acid amplification targeting ILV3 from a different genus or species is included in a separate reaction area. A reaction area is a defined location at which amplification takes place. It may be a well in a multi-well plate or a test tube for example. Sequencing, in particular next generation sequencing (NGS), may be utilised for detection and optionally also discrimination. Examples of NGS platforms include Illumina sequencing (such as Hi-Seq and Mi-Seq), SMRT sequencing (Pacific Biosciences), Nanopore sequencing, SoLID sequencing, pyrosequencing (e.g. Roche 454), single molecule sequencing (SeqLL/Helicos) and Ion-Torrent (Thermo Fisher) which are well-known to the skilled person and commercially available. As described herein, the ILV3 gene from different fungi has a different nucleotide sequence which can be probed using sequencing to identify the source of a fungal infection. Sequencing may provide rapid and quantitative results.

For simple detection, the mere presence of an amplification product indicates that there is a fungus present in the sample. As discussed herein, this is typically a fungus selected from Candida, Aspergillus and Cryptococcus neoformans. However, where more detailed information on the nature of the fungus is required the amplification products may be distinguished. In some embodiments, distinguishing involves a melt curve analysis. Various primer pairs described herein have been designed to have non-overlapping melt curves. Thus, when included in a multiplex amplification, the melt curve generated permits the species of Candida or Aspergillus in the sample to be identified. This may be a separate multiplex for Candida to the multiplex used for Aspergillus discrimination. It is shown herein that the methods of the invention based on a melt curve analysis permit discrimination of 8 different species of Candida and 3 different species of Aspergillus respectively. Melt curve analysis according to the invention may or may not rely upon use of sequence specific probes. In preferred embodiments, the methods do not require use of ILV3 specific probes. Instead a sequence independent reagent such as an intercalating agent, one example of which is SYBR GREEN, may be used to monitor amplification.

In other embodiments, amplification products may be distinguished by using differentially labelled primers and/or probes. In some embodiments, at least one primer and/or probe is differentially labelled according to genus to permit identification of the genus of fungus/yeast in the sample. In some embodiments, at least one primer and/or probe is differentially labelled according to species of Candida and/or Aspergillus to permit identification of the species of Candida and/or Aspergillus in the sample.

In still further embodiments, amplification products may be distinguished by determining the size of the amplification products. Primer pairs can be designed to amplify differently sized amplification products within the ILV3 gene of different genera and species if required.

In other embodiments, amplification products may be distinguished according to sequence.

The invention can advantageously be implemented in order to also detect bacteria in a sample. More specifically, the methods may further permit the determination of whether a bacteria or a fungus is present in the sample. In some embodiments, the methods may permit distinguishing whether the bacteria is Gram positive or Gram negative. Suitable reagents for such methods of detecting bacteria are disclosed in Klaschik et al (J. Clin. Microbiol. 2002,40(11):4304) and Wu et al (JOURNAL OF CLINICAL MICROBIOLOGY, August 2008, p. 2613-2619) each of which is hereby incorporated by reference. Such methods may rely upon use of a probe to distinguish Gram-negative from Gram-positive bacteria. In some embodiments, the primers amplify specific parts of the 16S region of bacterial DNA. The primers PLK1 (5-TACGGGAGGCAGCAGT-3—SEQ ID NO: 108) and PLK2 (5-TATTACCGC GGCTGCT-3—SEQ ID NO: 109) are highly conserved in different groups of eubacteria. A 187-bp fragment is synthesized by these primers. PLK2 may be labelled with fluorescein internally. The fluorescence dye-labelled hybridization probes ISN2 (5-CCGCAGAATAAG CACCGGCTAACTCCGT-3—SEQ ID NO: 110) and ISP2 (5-CCT AAC CAG AAA GCC ACG GCT AAC TAC GTG-3—SEQ ID NO: 111) emit light at different wavelengths (640 and 705 nm) and can be used for detection and Gram stain differentiation of bacterial DNA by a fluorescence signal. Other suitable primers may comprise the nucleotide sequence CAACGCGAAGAACCTTACC (SEQ ID NO: 112) and ACGTCATCCCCACCTTCC (SEQ ID NO: 113). A suitable Gram-positive probe comprises the nucleotide sequence 5′-FAM-ACGACAACCATGCACCACCTG-TAMRA-3′ (SEQ ID NO: 114). A suitable Gram-negative probe comprises the nucleotide sequence 5′-HEX-ACGACAGCCATGCAGCACCT-TAMRA′30 (SEQ ID NO: 115). Although these probes are differently labelled to permit differential detection, it will be appreciated by the skilled person that alternative approaches as described herein may be adopted to facilitate detection.

Thus, the invention further provides a method of detecting and identifying a microbial infection in a sample, comprising:
a. performing a nucleic acid amplification reaction comprising the following components:
   i. a forward and reverse primer hybridizing specifically to the 16S rRNA gene of Gram positive bacteria; optionally together with a probe that hybridizes between the primer binding sites specifically to the 16S rRNA gene of gram positive bacteria
   ii. a forward and reverse primer hybridizing specifically to the 16S rRNA gene of Gram negative bacteria; optionally together with a probe that hybridizes between the primer binding sites specifically to the 16S rRNA gene of Gram negative bacteria
   iii. a forward and reverse primer hybridizing specifically to the ILV3 gene of at least one fungal/yeast species; optionally together with a probe that hybridizes between the primer binding sites specifically to the ILV3 gene of at least one fungal/yeast species
b. detecting and distinguishing the amplification products to determine whether the sample contains a Gram negative bacterial infection, a Gram positive bacterial infection and/or a fungal/yeast infection.

Preferably, the amplification is performed as a multiplex although this is not essential as explained herein. ILV3 amplification may be performed according to any method of the invention or using any of the relevant primers and/or probes of the invention.

Corresponding kits are also provided. Thus, the invention provides a kit for discriminating a microbial infection in a sample, comprising components for performing a multiplex nucleic acid amplification reaction comprising:
a. a forward and reverse primer hybridizing specifically to the 16S rRNA gene of Gram positive bacteria; optionally together with a probe that hybridizes between the primer binding sites specifically to the 16S rRNA gene of Gram positive bacteria
b. a forward and reverse primer hybridizing specifically to the 16S rRNA gene of Gram negative bacteria; optionally together with a probe that hybridizes between the primer binding sites specifically to the 16S rRNA gene of Gram negative bacteria
c. a forward and reverse primer hybridizing specifically to the ILV3 gene of at least one fungal/yeast species; optionally together with a probe that hybridizes between the primer binding sites specifically to the ILV3 gene of at least one fungal/yeast species;
wherein components a, b and c each produce distinguishable amplification products thus enabling a determination of whether the sample contains a Gram negative bacterial infection, a Gram positive bacterial infection and/or a fungal/yeast infection.

Any suitable primer and probe according to the invention may be incorporated into such kits together with primers and probes for 16S rRNA amplification primers and probes. All embodiments of the invention discussed herein apply mutatis mutandis to these aspects of the invention. These methods may be followed by fungal species identification where needed. The kits may contain suitable components for this purpose as described herein.

The invention effectively provides for patient selection for therapy and, critically, avoids unnecessary treatment with antifungal agents such as fungicides (or antibiotics if bacteria are also detected). Incorrect use of antifungal agents and antibiotics fuels resistance.

Accordingly, the invention also relates to a method of selecting a subject for treatment with an antifungal agent such as a fungicide (or an antibiotic if bacteria are detected) comprising performing a method described herein and selecting the subject for treatment where an infection is detected, optionally also identified.

In a related aspect, the present invention provides a method of predicting responsiveness of a subject to treatment with an antifungal agent such as a fungicide (or an antibiotic if bacteria are detected) comprising performing a method described herein and predicting responsiveness of the subject to treatment where an infection is detected, optionally also identified.

In a further aspect the invention provides a method of treating an infection comprising administering an antifungal agent such as a fungicide (or an antibiotic if bacteria are detected) to the subject suffering from the infection, wherein the subject has been selected for treatment by performing a method described herein.

The invention also relates to a method of treating an infection comprising administering an antifungal agent such as a fungicide (or an antibiotic if bacteria are detected) to the subject suffering from the infection, wherein the subject displays, in a sample, a detectable ILV3 gene.

In yet a further aspect, the present invention provides an antifungal agent such as a fungicide (or an antibiotic if bacteria are detected) for use in a method of treating an infection, wherein the subject has been selected for treatment by performing the method described herein.

According to a further aspect of the invention there is provided an antifungal agent such as a fungicide for use in a method of treating an infection, wherein the subject displays, in a sample, a detectable ILV3 gene.

The infection may be a fungal or yeast infection, in particular a Candida, Aspergillus or Cryptococcus neoformans infection as explained herein in greater detail. This may direct the specifics of the treatment provided. For example, C. auris has been shown to be resistant to three main classes of antifungal drugs, including azoles (e.g. fluconazole). Similarly, species such as C. glabrata and C. krusei, may have a decreased susceptibility to anti-fungal agents such as fluconazole relative to other Candida species (Trick et al., 2002).

In certain embodiments the antifungal agent such as a fungicide (or an antibiotic if bacteria are detected) is a broad spectrum agent. This is particularly useful if an infection is detected but where the species responsible for the infection has not yet been characterised. Once the infection has been detected, the nature of the infection may be characterised so as to allow more targeted therapy (e.g. the species of Candida causing the infection). Thus, combinations of broad spectrum antifungal agents such as a fungicide (or an antibiotic if bacteria are detected) and more focused therapies may be employed as part of the methods described herein.

The invention can also be described by one or more of the following numbered clauses:
1. A method of detecting a fungal/yeast infection in a sample, comprising:
   a. performing a nucleic acid amplification reaction to amplify the ILV3 gene of fungi/yeast
   b. detecting the amplification product to determine whether the sample contains a fungal/yeast infection.
2. A method of detecting a fungal/yeast infection in a sample, comprising:
   a. performing a nucleic acid amplification reaction comprising the following components:
      i. a forward and reverse primer hybridizing specifically to the ILV3 gene of Candida species, optionally together with a probe that hybridizes between the primer binding sites specifically to the ILV3 gene of Candida species; and
      ii. a forward and reverse primer hybridizing specifically to the ILV3 gene of Aspergillus species, optionally together with a probe that hybridizes between the primer binding sites specifically to the ILV3 gene of Aspergillus species; and/or
      iii. a forward and reverse primer hybridizing specifically to the ILV3 gene of Cryptococcus neoformans, optionally together with a probe that hybridizes between the primer binding sites specifically to the ILV3 gene of Cryptococcus neoformans
   b. detecting the amplification products to determine whether the sample contains a fungal/yeast infection.
3. The method of clause 1 or 2 wherein the nucleic acid amplification reaction amplifies the ILV3 gene of at least 3, 4, 5, 6, 7, 8, 9, 10, 11 or all 12 of the following species:
   i. Candida albicans
   ii. Candida dubliniensis
   iii. Candida tropicalis
   iv. Candida parapsilosis
   v. Candida glabrata
   vi. Candida krusei
   vii. Candida guilliermondii
   viii. Candida auris
   ix. Aspergillus fumigatus
   x. Aspergillus niger
   xi. Aspergillus flavus
   xii. Cryptococcus neoformans.
4. The method of any one of clauses 1 to 3 wherein step a comprises:
   i. use of a forward and reverse primer hybridizing specifically to the ILV3 gene of Candida species; and/or
   ii. use of a probe that hybridizes specifically to the ILV3 gene of Candida species.
5. The method of any one of clauses 1 to 4 wherein a common forward and reverse primer and/or common probe hybridises to the ILV3 gene of at least 3, 4, 5, 6, 7 and preferably all, of the following Candida species:
   i. Candida albicans
   ii. Candida dubliniensis
   iii. Candida tropicalis
   iv. Candida parapsilosis
   v. Candida glabrata
   vi. Candida krusei
   vii. Candida guilliermondii
   viii. Candida auris
6. The method of clause 5 wherein the common forward primer hybridises to at least 3, 4, 5, 6, 7 and preferably all of the following target sequences:
   i. positions c1169825-1169806 of nucleotide sequence accession number NC_032093.1 (Candida albicans)
   ii. positions c393798-393779 of nucleotide sequence accession number NC_005968.1 (Candida glabrata)
   iii. positions c405996-405978 of nucleotide sequence accession number NW_003020040.1 (Candida tropicalis)
   iv. positions c1218036-1218017 of nucleotide sequence accession number NC_012864.1 (Candida dubliniensis)
   v. positions c909809-909790 of nucleotide sequence accession number NW_001809800.1 (Candida guilliermondii)
   vi. positions c24006-23987 of nucleotide sequence accession number HE605203.1 (Candida parapsilosis)
   vii. positions 61666-61685 of nucleotide sequence accession number JQFK01000016.1 (Candida krusei)
   viii. positions c32324-32305 of nucleotide sequence accession number NW_017263971.1 (Candida auris).
7. The method of clause 5 or 6 wherein the common reverse primer hybridises to at least 3, 4, 5, 6, 7 and preferably all of the following target sequences:
   i. positions 1169707-1169729 of nucleotide sequence accession number NC_032093.1 (Candida albicans)
   ii. positions 393680-393702 of nucleotide sequence accession number NC_005968.1 (Candida glabrata)
   iii. positions 405878-405900 of nucleotide sequence accession number NW_003020040.1 (Candida tropicalis)
   iv. positions 1217918-1217940 of nucleotide sequence accession number NC_012864.1 (Candida dubliniensis)
   v. positions 909691-909713 of nucleotide sequence accession number NW_001809800.1 (Candida guilliermondii)
   vi. positions 23888-23910 of nucleotide sequence accession number HE605203.1 (Candida parapsilosis)
   vii. positions c61784-61762 of nucleotide sequence accession number JQFK01000016.1 (Candida krusei)
   viii. positions 32206-32228 of nucleotide sequence accession number NW_017263971.1 (Candida auris).
8. The method of any one of clauses 5 to 7 wherein the common probe hybridises to at least 3, 4, 5, 6, 7 and preferably all of the following target sequences:
   i. positions 1169779-1169804 of nucleotide sequence accession number NC_032093.1 (Candida albicans)
   ii. positions 393752-393777 of nucleotide sequence accession number NC_005968.1 (Candida glabrata)
   iii. positions 405950-405975 of nucleotide sequence accession number NW_003020040.1 (Candida tropicalis)
   iv. positions 1217990-1218015 of nucleotide sequence accession number NC_012864.1 (Candida dubliniensis)
   v. positions 909763-909788 of nucleotide sequence accession number NW_001809800.1 (Candida guilliermondii)
   vi. positions 23960-23985 of nucleotide sequence accession number HE605203.1 (Candida parapsilosis)
   vii. positions c61712-61687 of nucleotide sequence accession number JQFK01000016.1 (Candida krusei)
   viii. positions 32278-32303 of nucleotide sequence accession number NW_017263971.1 (Candida auris).
9. The method of any one of clauses 1 to 8 which uses a forward primer comprising the sequence of SEQ ID NO: 1, a reverse primer comprising the sequence of SEQ ID NO: 2 and/or a probe comprising the sequence of SEQ ID NO: 3.
10. The method of any one of clauses 1 to 3 wherein a separate forward and reverse primer and/or probe hybridises to the ILV3 gene of each of at least 3, 4, 5, 6, 7 and preferably all, of the following Candida species:
   i. Candida albicans
   ii. Candida dubliniensis
   iii. Candida tropicalis
   iv. Candida parapsilosis
   v. Candida glabrata
   vi. Candida krusei
   vii. Candida guilliermondii
   viii. Candida auris
11. The method of clause 10 wherein the forward primer hybridizing specifically to the ILV3 gene of Candida albicans hybridises to one of the following target sequences from nucleotide sequence accession number NC_032093.1:
   i. positions c1170234-1170217
   ii. positions c1171213-1171192
12. The method of clause 10 or 11 wherein the reverse primer hybridizing specifically to the ILV3 gene of Candida albicans hybridises to one of the following target sequences from nucleotide sequence accession number NC_032093.1:
   i. positions 1170119-1170140
   ii. positions 1171120-1171138
13. The method of any one of clauses 10 to 12 wherein the forward primer hybridizing specifically to the ILV3 gene of Candida dubliniensis hybridises to one of the following target sequences from nucleotide sequence accession number NC_012864.1:
   i. positions c1219181-1219161
   ii. positions c1218505-1218486
   iii. positions c1218553-1218534
14. The method of any one of clauses 10 to 13 wherein the reverse primer hybridizing specifically to the ILV3 gene of Candida dubliniensis hybridises to one of the following target sequences from nucleotide sequence accession number NC_012864.1:
   i. positions 1219103-1219124
   ii. positions 1218409-1218429
   iii. positions 1218419-1218440
15. The method of any one of clauses 10 to 14 wherein the forward primer hybridizing specifically to the ILV3 gene of Candida tropicalis hybridises to one of the following target sequences from nucleotide sequence accession number NW_003020040.1:
   i. positions c406146-406127
   ii. positions c406142-406120
16. The method of any one of clauses 10 to 15 wherein the reverse primer hybridizing specifically to the ILV3 gene of Candida tropicalis hybridises to one of the following target sequences from nucleotide sequence accession number NW_003020040.1:
   i. positions 406047-406068
   ii. positions 406048-406069
17. The method of any one of clauses 10 to 16 wherein the forward primer hybridizing specifically to the ILV3 gene of Candida parapsilosis hybridises to one of the following target sequences from nucleotide sequence accession number HE605203.1:
   i. positions c24951-24931
   ii. positions c24881-24863
18. The method of any one of clauses 10 to 17 wherein the reverse primer hybridizing specifically to the ILV3 gene of Candida parapsilosis hybridises to one of the following target sequences from nucleotide sequence accession number HE605203.1:
   i. positions 24843-24865
   ii. positions 24774-24795
19. The method of any one of clauses 10 to 18 wherein the forward primer hybridizing specifically to the ILV3 gene of Candida glabrata hybridises to one of the following target sequences from nucleotide sequence accession number NC_005968.1:
   i. positions c394191-394169
   ii. positions c393535-393516
   iii. positions c395076-395055
   iv. positions c394884-394863
20. The method of any one of clauses 10 to 19 wherein the reverse primer hybridizing specifically to the ILV3 gene of Candida glabrata hybridises to one of the following target sequences from nucleotide sequence accession number NC_005968.1:
   i. positions 394080-394101
   ii. positions 393445-393464
   iii. positions 394987-395006
   iv. positions 394783-394803
21. The method of any one of clauses 10 to 20 wherein the forward primer hybridizing specifically to the ILV3 gene of Candida krusei hybridises to one of the following target sequences from nucleotide sequence accession number JQFK01000016.1:
   i. positions 61723-61744
   ii. positions 60940-60961
   iii. positions 60420-60440
   iv. positions 61778-61797
   v. positions 61940-61961
22. The method of any one of clauses 10 to 21 wherein the reverse primer hybridizing specifically to the ILV3 gene of Candida krusei hybridises to one of the following target sequences from nucleotide sequence accession number JQFK01000016.1:
   i. positions c61817-61796
   ii. positions c61030-61011
   iii. positions c60535-60513
   iv. positions c61923-61902
   v. positions c62015-61996
23. The method of any one of clauses 10 to 22 wherein the forward primer hybridizing specifically to the ILV3 gene of Candida guilliermondii hybridises to one of the following target sequences from nucleotide sequence accession number NW_001809800.1:
   i. positions c909629-909608
   ii. positions c911117-911098
   iii. positions c911111-911090
   iv. positions c910941-910920
24. The method of any one of clauses 10 to 23 wherein the reverse primer hybridizing specifically to the ILV3 gene of Candida guilliermondii hybridises to one of the following target sequences from nucleotide sequence accession number NW_001809800.1:
   i. positions 909529-909550
   ii. positions 911002-911023
   iii. positions 910994-911013
   iv. positions 910799-910819
25. The method of any one of clauses 10 to 24 wherein the forward primer hybridizing specifically to the ILV3 gene of Candida auris hybridises to one of the following target sequences from nucleotide sequence accession number NW_017263971.1:
   i. positions c32790-32768
   ii. positions c32451-32430
   iii. positions c32654-32633
   iv. positions c33278-33259
   v. positions c32603-32580
   vi. positions c32399-32376
   vii. positions c33648-33628
   viii. positions c32240-32219
   ix. positions c32930-32909
   x. positions c33228-33206
   xi. positions c32106-32085
26. The method of any one of clauses 10 to 25 wherein the reverse primer hybridizing specifically to the ILV3 gene of Candida auris hybridises to one of the following target sequences from nucleotide sequence accession number NW_017263971.1:
   i. positions 32682-32703
   ii. positions 32368-32389
   iii. positions 32567-32588
   iv. positions 33202-33223
   v. positions 32512-32533
   vi. positions 32328-32349
   vii. positions 33566-33587
   viii. positions 32175-32195
   ix. positions 32826-32848
   x. positions 33130-33151
   xi. positions 32011-32033
27. The method of any one of clauses 10 to 26 wherein:
   a. the probe hybridizing specifically to the ILV3 gene of Candida albicans hybridises to one of the following target sequences from nucleotide sequence accession number NC_032093.1:
      i. positions c1170174-1170151
      ii. positions 1171165-1171192
   b. the probe hybridizing specifically to the ILV3 gene of Candida dubliniensis hybridises to one of the following target sequences from nucleotide sequence accession number NC_012864.1:
      i. positions 1219148-1219171
      ii. positions c1218439-1218416
      iii. positions c1218505-1218480;
   c. the probe hybridizing specifically to the ILV3 gene of Candida tropicalis hybridises to one of the following target sequences from nucleotide sequence accession number NW_003020040.1:
      i. positions c406111-406082;
   d. the probe hybridizing specifically to the ILV3 gene of Candida parapsilosis hybridises to one of the following target sequences from nucleotide sequence accession number HE605203.1:
      i. positions c24907-24883
      ii. positions c24862-24839
   e. the probe hybridizing specifically to the ILV3 gene of Candida glabrata hybridises to one of the following target sequences from nucleotide sequence accession number NC_005968.1:
      i. positions c394151-394128
      ii. positions c393478-393453
      iii. positions c395030-395007
      iv. positions c394836-394807
   f. the probe hybridizing specifically to the ILV3 gene of Candida krusei hybridises to one of the following target sequences from nucleotide sequence accession number JQFK01000016.1:
      i. positions 61770-61793
      ii. positions c60990-60967
      iii. positions 60462-60485
      iv. positions 61815-61838
      v. positions c61970-61945
   g. the probe hybridizing specifically to the ILV3 gene of Candida guilliermondii hybridises to one of the following target sequences from nucleotide sequence accession number NW_001809800.1:
      i. positions c909577-909552
      ii. positions c911088-911065
      iii. positions 910878-910904; and/or
   h. the probe hybridizing specifically to the ILV3 gene of Candida auris hybridises to one of the following target sequences from nucleotide sequence accession number NW_017263971.1:
      i. positions c32744-32721
      ii. positions c32421-32398
      iii. positions c32629-32605
      iv. positions 33233-33257
      v. positions 32542-32563
      vi. positions 32350-32373
      vii. positions c33627-33606
      viii. positions c32218-32196
      ix. positions c32890-32864
      x. positions c33259-33236
      xi. positions c32061-32038
28. The method of any one of clauses 10 to 27 wherein:
   i. the forward primer comprises the sequence of: SEQ ID NO: 4 or 6, the reverse primer comprises the sequence of SEQ ID NO: 5 or 7 and/or the probe comprises the sequence of: SEQ ID NO: 116 or 117 to specifically detect Candida albicans
   ii. the forward primer comprises the sequence of: SEQ ID NO: 8, 10 or 12, the reverse primer comprises the sequence of SEQ ID NO: 9, 11 or 13 and/or the probe comprises the sequence of: SEQ ID NO: 118, 119 or 120 to specifically detect Candida dubliniensis
   iii. the forward primer comprises the sequence of: SEQ ID NO: 14 or 16, the reverse primer comprises the sequence of SEQ ID NO: 15 or 17 and/or the probe comprises the sequence of: SEQ ID NO: 121 to specifically detect Candida tropicalis
   iv. the forward primer comprises the sequence of: SEQ ID NO: 18 or 20, the reverse primer comprises the sequence of SEQ ID NO: 19 or 21 and/or the probe comprises the sequence of: SEQ ID NO: 122 or 123 to specifically detect Candida parapsilosis
   v. the forward primer comprises the sequence of: SEQ ID NO: 22, 24, 26 or 28, the reverse primer comprises the sequence of SEQ ID NO: 23, 25, 27 or 29 and/or the probe comprises the sequence of: SEQ ID NO: 124, 125, 126 or 127 to specifically detect Candida glabrata
   vi. the forward primer comprises the sequence of: SEQ ID NO: 30, 32, 34, 36 or 38, the reverse primer comprises the sequence of SEQ ID NO: 31, 33, 35, 37 or 39 and/or the probe comprises the sequence of: SEQ ID NO: 128, 129, 130, 131 or 132 to specifically detect Candida krusei
   vii. the forward primer comprises the sequence of: SEQ ID NO: 40, 42, 44 or 46, the reverse primer comprises the sequence of SEQ ID NO: 41, 43, 45 or 47 and/or the probe comprises the sequence of: SEQ ID NO: 133, 134 or 135 to specifically detect Candida guilliermondii
   viii. the forward primer comprises the sequence of: SEQ ID NO: 48, 50, 52, 54, 56, 58, 60, 62, 64, 66 or 68, the reverse primer comprises the sequence of SEQ ID NO: 49, 51, 53, 55, 57, 59, 61, 63, 65, 67 or 69 and/or the probe comprises the sequence of: SEQ ID NO: 136, 137, 138, 139, 140, 141, 142, 143, 144, 145 or 146 to specifically detect Candida auris.
29. The method of any one of clauses 1 to 28 wherein step a comprises:
   i. use of a forward and reverse primer hybridizing specifically to the ILV3 gene of Aspergillus species; and/or
   ii. use of a probe that hybridizes specifically to the ILV3 gene of Aspergillus species.
30. The method of clause 29 wherein a common forward and reverse primer and/or probe hybridises to the ILV3 gene of at least 2, and preferably all 3, of the following Aspergillus species:
   i. Aspergillus fumigatus
   ii. Aspergillus niger
   iii. Aspergillus flavus
31. The method of clause 30 wherein the common forward primer hybridises to at least 2, and preferably all 3, of the following target sequences:
   i. positions 3721583-3721597 of nucleotide sequence accession number NC_007195.1 (Aspergillus fumigatus)
   ii. positions c541018-541004 of nucleotide sequence accession number NT_166533.1 (Aspergillus niger)
   iii. positions c382612-382598 of nucleotide sequence accession number NW_002477240.1 (Aspergillus flavus)
      or wherein the common forward primer hybridises to at least 2, and preferably all 3, of the following target sequences:
   i. positions 3721797-3721816 of nucleotide sequence accession number NC_007195.1 (Aspergillus fumigatus)
   ii. positions c540804-540785 of nucleotide sequence accession number NT_166533.1 (Aspergillus niger)
   iii. positions c382398-382379 of nucleotide sequence accession number NW_002477240.1 (Aspergillus flavus)
32. The method of clause 30 or 31 wherein the common reverse primer hybridises to at least 2, and preferably all 3, of the following target sequences:
   i. positions c3721887-3721872 of nucleotide sequence accession number NC_007195.1 (Aspergillus fumigatus)
   ii. positions 540714-540729 of nucleotide sequence accession number NT_166533.1 (Aspergillus niger)
   iii. positions 382308-382323 of nucleotide sequence accession number NW_002477240.1 (Aspergillus flavus)
      or wherein the common reverse primer hybridises to at least 2, and preferably all 3, of the following target sequences:
   i. positions c3721875-3721894 of nucleotide sequence accession number NC_007195.1 (Aspergillus fumigatus)
   ii. positions 540707-540726 of nucleotide sequence accession number NT_166533.1 (Aspergillus niger)
   iii. positions 382301-382320 of nucleotide sequence accession number NW_002477240.1 (Aspergillus flavus)
33. The method of any one of clauses 30 to 32 wherein the common probe hybridises to at least 2, and preferably all 3, of the following target sequences:
   i. positions 3721790-3721814 of nucleotide sequence accession number NC_007195.1 (Aspergillus fumigatus)
   ii. positions c540811-540788 of nucleotide sequence accession number NT_166533.1 (Aspergillus niger)
   iii. positions c382405-382381 of nucleotide sequence accession number NW_002477240.1 (Aspergillus flavus)
      or wherein the common probe hybridises to at least 2, and preferably all 3, of the following target sequences:
   iv. positions 3721848-3721870 of nucleotide sequence accession number NC_007195.1 (Aspergillus fumigatus)
   v. positions c540753-540731 of nucleotide sequence accession number NT_166533.1 (Aspergillus niger)
   vi. positions c382347-382325 of nucleotide sequence accession number NW_002477240.1 (Aspergillus flavus).
34. The method of any one of clauses 1 to 33 which uses a forward primer comprising the sequence of SEQ ID NO: 70 or 73, the reverse primer comprises the sequence of SEQ ID NO: 71 or 74 and/or the probe comprises the sequence of SEQ ID NO: 72 or 75.
35. The method of any one of clauses 1 to 34 wherein a separate forward and reverse primer and/or probe hybridises to the ILV3 gene of each of at least 2, and preferably all 3, of the following Aspergillus species:
   i. Aspergillus fumigatus
   ii. Aspergillus niger
   iii. Aspergillus flavus.
36. The method of clause 35 wherein the forward primer hybridizing specifically to the ILV3 gene of Aspergillus fumigatus hybridises to one of the following target sequences from nucleotide sequence accession number NC_007195.1:
   i. positions 3721531-3721552
   ii. positions 3721618-3721638
   iii. positions 3721616-3721638
   iv. positions 3721798-3721818
37. The method of clause 35 or 36 wherein the reverse primer hybridizing specifically to the ILV3 gene of Aspergillus fumigatus hybridises to one of the following target sequences from nucleotide sequence accession number NC_007195.1:
   i. positions c3721653-3721675
   ii. positions c3721706-3721681
   iii. positions c3721898-3721878
38. The method of any one of clauses 35 to 37 wherein the forward primer hybridizing specifically to the ILV3 gene of Aspergillus niger hybridises to one of the following target sequences from nucleotide sequence accession number NT_166533.1:
   i. positions c540219-540199
39. The method of any one of clauses 35 to 38 wherein the reverse primer hybridizing specifically to the ILV3 gene of Aspergillus niger hybridises to one of the following target sequences from nucleotide sequence accession number NT_166533.1:
   i. positions 540084-540103
40. The method of any one of clauses 35 to 39 wherein the forward primer hybridizing specifically to the ILV3 gene of Aspergillus flavus hybridises to one of the following target sequences from nucleotide sequence accession number NW_002477240.1:
   i. positions c382604-382586
41. The method of any one of clauses 35 to 40 wherein the reverse primer hybridizing specifically to the ILV3 gene of Aspergillus flavus hybridises to one of the following target sequences from nucleotide sequence accession number NW_002477240.1:
   i. positions 382519-382541
   ii. positions 382520-382541
42. The method of any one of clauses 35 to 41 wherein:
   a. the probe hybridizing specifically to the ILV3 gene of Aspergillus fumigatus hybridises to one of the following target sequences from nucleotide sequence accession number NC_007195.1:
   i. positions c3721651-3721628
   ii. positions 3721658-3721681
   iii. positions 3721848-3721870
   b. the probe hybridizing specifically to the ILV3 gene of Aspergillus niger hybridises to one of the following target sequences from nucleotide sequence accession number NT_166533.1:
   i. positions c540161-540138; and/or
   c. the probe hybridizing specifically to the ILV3 gene of Aspergillus flavus hybridises to one of the following target sequences from nucleotide sequence accession number NW_002477240.1:
   i. positions c382572-382549
43. The method of any one of clauses 35 to 42 wherein:
   i. the forward primer comprises the sequence of: SEQ ID NO: 76, 79, 82 or 83, the reverse primer comprises the sequence of SEQ ID NO: 77, 80 or 84 and/or the probe comprises the sequence of SEQ ID NO: 78, 81 or 85 to specifically detect Aspergillus fumigatus
   ii. the forward primer comprises the sequence of: SEQ ID NO: 87, the reverse primer comprises the sequence of SEQ ID NO: 86 and/or the probe comprises the sequence of SEQ ID NO: 88 to specifically detect Aspergillus niger
   iii. the forward primer comprises the sequence of: SEQ ID NO: 90, the reverse primer comprises the sequence of SEQ ID NO: 89 or 92 and/or the probe comprises the sequence of SEQ ID NO: 91 to specifically detect Aspergillus flavus.
44. The method of any one of clauses 1 to 43 wherein step a comprises:
   i. use of a forward and reverse primer hybridizing specifically to the ILV3 gene of Cryptococcus neoformans; and/or
   ii. use of a probe that hybridizes specifically to the ILV3 gene of Cryptococcus neoformans.
45. The method of clause 44 wherein the forward primer hybridizing specifically to the ILV3 gene of Cryptococcus neoformans hybridises to one of the following target sequences from nucleotide sequence accession number NC_006693.1:
   i. Positions 702696-702717
   ii. Positions 702499-702520
   iii. Positions 702736-702757
   iv. Positions 702384-702403
   v. Positions 701384-701406
46. The method of clause 44 or 45 wherein the reverse primer hybridizing specifically to the ILV3 gene of Cryptococcus neoformans hybridises to one of the following target sequences from nucleotide sequence accession number NC_006693.1:
   i. Positions c702796-702777
   ii. Positions c702602-702582
   iii. Positions c702850-702831
   iv. Positions c702521-702500
   v. Positions c701499-701479
47. The method of any one of clauses 44 to 46 wherein the probe that hybridizes between the primer binding sites specifically to the ILV3 gene of Cryptococcus neoformans hybridises to one of the following target sequences from nucleotide sequence accession number NC_006693.1:
   i. Positions 702724-702747
   ii. Positions 702526-702549
   iii. Positions 702805-702829
   iv. Positions 702423-702446
   v. Positions 701435-701458
48. The method of any one of clauses 44 to 47 wherein the forward primer comprises the sequence of SEQ ID NO: 93, 96, 99, 102 or 105, the reverse primer comprises the sequence of SEQ ID NO: 94, 97, 100, 103 or 106 and/or the probe comprises the sequence of SEQ ID NO: 95, 98, 101, 104 or 107.
49. The method of any preceding clause wherein step b comprises distinguishing amplification products in order to identify the genus and/or species responsible for the infection.
50. A method of detecting and identifying a fungal/yeast infection in a sample, comprising performing the method of any preceding clause and, in step b, detecting and distinguishing the amplification products to identify the fungal/yeast infection.
51. A method of detecting and identifying a fungal/yeast infection in a sample, comprising:
   a. performing a nucleic acid amplification reaction comprising the following components:
      i. a forward and reverse primer hybridizing specifically to the ILV3 gene of Candida species, optionally together with a probe that hybridizes between the primer binding sites specifically to the ILV3 gene of Candida species; and
      ii. a forward and reverse primer hybridizing specifically to the ILV3 gene of Aspergillus species, optionally together with a probe that hybridizes between the primer binding sites specifically to the ILV3 gene of Aspergillus species; and/or
      iii. a forward and reverse primer hybridizing specifically to the ILV3 gene of Cryptococcus neoformans, optionally together with a probe that hybridizes between the primer binding sites specifically to the ILV3 gene of Cryptococcus neoformans
   b. detecting and distinguishing the amplification products to identify the fungal/yeast infection.
52. The method of any preceding clause which is performed as a multiplex nucleic acid amplification reaction.
53. The method of any one of clauses 49 to 52 wherein distinguishing comprises:
   i. a melting curve analysis
   ii. use of differently labelled primers and/or probes; or
   iii. determining the size of the amplification products.
54. The method of clause 53 wherein at least one primer and/or probe is differentially labelled according to genus to permit identification of the genus of fungus/yeast in the sample.
55. The method of clause 54 wherein at least one primer and/or probe is differentially labelled according to species of Candida and/or Aspergillus to permit identification of the species of Candida and/or Aspergillus in the sample.
56. The method of any preceding clause wherein the sample comprises a test sample from a human, optionally a blood sample.
57. At least one primer pair for detecting a yeast/fungus infection in a sample comprising:
   a. a forward and reverse primer hybridizing specifically to the ILV3 gene of the following Candida species
      i. Candida albicans
      ii. Candida dubliniensis
      iii. Candida tropicalis
      iv. Candida parapsilosis
      v. Candida glabrata
      vi. Candida krusei
      vii. Candida guilliermondii
      viii. Candida auris
   b. a forward and reverse primer hybridizing specifically to the ILV3 gene of the following Aspergillus species
      i. Aspergillus fumigatus
      ii. Aspergillus niger
      iii. Aspergillus flavus
   c. a forward and reverse primer hybridizing specifically to the ILV3 gene of Candida albicans
   d. a forward and reverse primer hybridizing specifically to the ILV3 gene of Candida dubliniensis
   e. a forward and reverse primer hybridizing specifically to the ILV3 gene of Candida tropicalis
   f. a forward and reverse primer hybridizing specifically to the ILV3 gene of Candida parapsilosis
   g. a forward and reverse primer hybridizing specifically to the ILV3 gene of Candida glabrata
   h. a forward and reverse primer hybridizing specifically to the ILV3 gene of Candida krusei
   i. a forward and reverse primer hybridizing specifically to the ILV3 gene of Candida guilliermondii
   j. a forward and reverse primer hybridizing specifically to the ILV3 gene of Candida auris
   k. a forward and reverse primer hybridizing specifically to the ILV3 gene of Aspergillus fumigatus;
   l. a forward and reverse primer hybridizing specifically to the ILV3 gene of Aspergillus niger;
   m. a forward and reverse primer hybridizing specifically to the ILV3 gene of Aspergillus flavus; and/or
   n. a forward and reverse primer hybridizing specifically to the ILV3 gene of Cryptococcus neoformans.
58. The at least one primer pair of clause 57 wherein the forward primer of a primer pair hybridises to at least 3, 4, 5, 6, 7 and preferably all of the following target sequences:
   i. positions c1169825-1169806 of nucleotide sequence accession number NC_032093.1 (Candida albicans)
   ii. positions c393798-393779 of nucleotide sequence accession number NC_005968.1 (Candida glabrata)
   iii. positions c405996-405978 of nucleotide sequence accession number NW_003020040.1 (Candida tropicalis)
   iv. positions c1218036-1218017 of nucleotide sequence accession number NC_012864.1 (Candida dubliniensis)
   v. positions c909809-909790 of nucleotide sequence accession number NW_001809800.1 (Candida guilliermondii)
   vi. positions c24006-23987 of nucleotide sequence accession number HE605203.1 (Candida parapsilosis)
   vii. positions 61666-61685 of nucleotide sequence accession number JQFK01000016.1 (Candida krusei)
   viii. positions c32324-32305 of nucleotide sequence accession number NW_017263971.1 (Candida auris).
59. The at least one primer pair of clause 57 or 58 wherein the reverse primer of a primer pair hybridises to at least 3, 4, 5, 6, 7 and preferably all of the following target sequences:
   i. positions 1169707-1169729 of nucleotide sequence accession number NC_032093.1 (Candida albicans)
   ii. positions 393680-393702 of nucleotide sequence accession number NC_005968.1 (Candida glabrata)
   iii. positions 405878-405900 of nucleotide sequence accession number NW_003020040.1 (Candida tropicalis)
   iv. positions 1217918-1217940 of nucleotide sequence accession number NC_012864.1 (Candida dubliniensis)
   v. positions 909691-909713 of nucleotide sequence accession number NW_001809800.1 (Candida guilliermondii)
   vi. positions 23888-23910 of nucleotide sequence accession number HE605203.1 (Candida parapsilosis)
   vii. positions c61784-61762 of nucleotide sequence accession number JQFK01000016.1 (Candida krusei)
   viii. positions 32206-32228 of nucleotide sequence accession number NW_017263971.1 (Candida auris).
60. The at least one primer pair of any one of clauses 57 to 59 wherein the forward primer of a primer pair hybridises to at least 2, and preferably all 3, of the following target sequences:
   i. positions 3721583-3721597 of nucleotide sequence accession number NC_007195.1 (Aspergillus fumigatus)
   ii. positions c541018-541004 of nucleotide sequence accession number NT_166533.1 (Aspergillus niger)
   iii. positions c382612-382598 of nucleotide sequence accession number NW_002477240.1 (Aspergillus flavus)
      or wherein the forward primer hybridises to at least 2, and preferably all 3, of the following target sequences:
   iv. positions 3721797-3721816 of nucleotide sequence accession number NC_007195.1 (Aspergillus fumigatus)
   v. positions c540804-540785 of nucleotide sequence accession number NT_166533.1 (Aspergillus niger)
   vi. positions c382398-382379 of nucleotide sequence accession number NW_002477240.1 (Aspergillus flavus)
61. The at least one primer pair of any one of clauses 57 to 60 wherein the reverse primer of a primer pair hybridises to at least 2, and preferably all 3, of the following target sequences:
   i. positions c3721887-3721872 of nucleotide sequence accession number NC_007195.1 (Aspergillus fumigatus)
   ii. positions 540714-540729 of nucleotide sequence accession number NT_166533.1 (Aspergillus niger)
   iii. positions 382308-382323 of nucleotide sequence accession number NW_002477240.1 (Aspergillus flavus)
      or wherein the reverse primer hybridises to at least 2, and preferably all 3, of the following target sequences:
   iv. positions c3721875-3721894 of nucleotide sequence accession number NC_007195.1 (Aspergillus fumigatus)
   v. positions 540707-540726 of nucleotide sequence accession number NT_166533.1 (Aspergillus niger)
   vi. positions 382301-382320 of nucleotide sequence accession number NW_002477240.1 (Aspergillus flavus)
62. The at least one primer pair of any one of clauses 57 to 61 wherein the forward primer of a primer pair hybridizing specifically to the ILV3 gene of Candida albicans hybridises to one of the following target sequences from nucleotide sequence accession number NC_032093.1:
   i. positions c1170234-1170217
   ii. positions c1171213-1171192
63. The at least one primer pair of any one of clauses 57 to 62 wherein the reverse primer of a primer pair hybridizing specifically to the ILV3 gene of Candida albicans hybridises to one of the following target sequences from nucleotide sequence accession number NC_032093.1:
   i. positions 1170119-1170140
   ii. positions 1171120-1171138
64. The at least one primer pair of any one of clauses 57 to 63 wherein the forward primer of a primer pair hybridizing specifically to the ILV3 gene of Candida dubliniensis hybridises to one of the following target sequences from nucleotide sequence accession number NC_012864.1:
   i. positions c1219181-1219161
   ii. positions c1218505-1218486
   iii. positions c1218553-1218534
65. The at least one primer pair of any one of clauses 57 to 64 wherein the reverse primer of a primer pair hybridizing specifically to the ILV3 gene of Candida dubliniensis hybridises to one of the following target sequences from nucleotide sequence accession number NC_012864.1:
   i. positions 1219103-1219124
   ii. positions 1218409-1218429
   iii. positions 1218419-1218440
66. The at least one primer pair of any one of clauses 57 to 65 wherein the forward primer of a primer pair hybridizing specifically to the ILV3 gene of Candida tropicalis hybridises to one of the following target sequences from nucleotide sequence accession number NW_003020040.1:
   i. positions c406146-406127
   ii. positions c406142-406120
67. The at least one primer pair of any one of clauses 57 to 66 wherein the reverse primer of a primer pair hybridizing specifically to the ILV3 gene of Candida tropicalis hybridises to one of the following target sequences from nucleotide sequence accession number NW_003020040.1:
   i. positions 406047-406068
   ii. positions 406048-406069
68. The at least one primer pair of any one of clauses 57 to 67 wherein the forward primer of a primer pair hybridizing specifically to the ILV3 gene of Candida parapsilosis hybridises to one of the following target sequences from nucleotide sequence accession number HE605203.1:
   i. positions c24951-24931
   ii. positions c24881-24863
69. The at least one primer pair of any one of clauses 57 to 68 wherein the reverse primer of a primer pair hybridizing specifically to the ILV3 gene of Candida parapsilosis hybridises to one of the following target sequences from nucleotide sequence accession number HE605203.1:
   i. positions 24843-24865
   ii. positions 24774-24795
70. The at least one primer pair of any one of clauses 57 to 69 wherein the forward primer of a primer pair hybridizing specifically to the ILV3 gene of Candida glabrata hybridises to one of the following target sequences from nucleotide sequence accession number NC_005968.1:
   i. positions c394191-394169
   ii. positions c393535-393516
   iii. positions c395076-395055
   iv. positions c394884-394863
71. The at least one primer pair of any one of clauses 57 to 70 wherein the reverse primer of a primer pair hybridizing specifically to the ILV3 gene of Candida glabrata hybridises to one of the following target sequences from nucleotide sequence accession number NC_005968.1:
   i. positions 394080-394101
   ii. positions 393445-393464
   iii. positions 394987-395006
   iv. positions 394783-394803
72. The at least one primer pair of any one of clauses 57 to 71 wherein the forward primer of a primer pair hybridizing specifically to the ILV3 gene of Candida krusei hybridises to one of the following target sequences from nucleotide sequence accession number JQFK01000016.1:
   i. positions 61723-61744
   ii. positions 60940-60961
   iii. positions 60420-60440
   iv. positions 61778-61797
   v. positions 61940-61961
73. The at least one primer pair of any one of clauses 57 to 72 wherein the reverse primer of a primer pair hybridizing specifically to the ILV3 gene of Candida krusei hybridises to one of the following target sequences from nucleotide sequence accession number JQFK01000016.1:
   i. positions c61817-61796
   ii. positions c61030-61011
   iii. positions c60535-60513
   iv. positions c61923-61902
   v. positions c62015-61996
74. The at least one primer pair of any one of clauses 57 to 73 wherein the forward primer of a primer pair hybridizing specifically to the ILV3 gene of Candida guilliermondii hybridises to one of the following target sequences from nucleotide sequence accession number NW_001809800.1:
   i. positions c909629-909608
   ii. positions c911117-911098
   iii. positions c911111-911090
   iv. positions c910941-910920
75. The at least one primer pair of any one of clauses 57 to 74 wherein the reverse primer of a primer pair hybridizing specifically to the ILV3 gene of Candida guilliermondii hybridises to one of the following target sequences from nucleotide sequence accession number NW_001809800.1:
   i. positions 909529-909550
   ii. positions 911002-911023
   iii. positions 910994-911013
   iv. positions 910799-910819
76. The at least one primer pair of any one of clauses 57 to 75 wherein the forward primer of a primer pair hybridizing specifically to the ILV3 gene of Candida auris hybridises to one of the following target sequences from nucleotide sequence accession number NW_017263971.1:
   i. positions c32790-32768
   ii. positions c32451-32430
   iii. positions c32654-32633
   iv. positions c33278-33259
   v. positions c32603-32580
   vi. positions c32399-32376
   vii. positions c33648-33628
   viii. positions c32240-32219
   ix. positions c32930-32909
   x. positions c33228-33206
   xi. positions c32106-32085
77. The at least one primer pair of any one of clauses 57 to 76 wherein the reverse primer of a primer pair hybridizing specifically to the ILV3 gene of Candida auris hybridises to one of the following target sequences from nucleotide sequence accession number NW_017263971.1:
   i. positions 32682-32703
   ii. positions 32368-32389
   iii. positions 32567-32588
   iv. positions 33202-33223
   v. positions 32512-32533
   vi. positions 32328-32349
   vii. positions 33566-33587
   viii. positions 32175-32195
   ix. positions 32826-32848
   x. positions 33130-33151
   xi. positions 32011-32033
78. The at least one primer pair of any one of clauses 57 to 77 wherein the forward primer of a primer pair hybridizing specifically to the ILV3 gene of Aspergillus fumigatus hybridises to one of the following target sequences from nucleotide sequence accession number NC_007195.1:
   i. positions 3721531-3721552
   ii. positions 3721618-3721638
   iii. positions 3721616-3721638
   iv. positions 3721798-3721818
79. The at least one primer pair of any one of clauses 57 to 78 wherein the reverse primer of a primer pair hybridizing specifically to the ILV3 gene of Aspergillus fumigatus hybridises to one of the following target sequences from nucleotide sequence accession number NC_007195.1:
   i. positions c3721653-3721675
   ii. positions c3721706-3721681
   iii. positions c3721898-3721878
80. The at least one primer pair of any one of clauses 57 to 79 wherein the forward primer of a primer pair hybridizing specifically to the ILV3 gene of Aspergillus niger hybridises to one of the following target sequences from nucleotide sequence accession number NT_166533.1:
   i. positions c540219-540199
81. The at least one primer pair of any one of clauses 57 to 80 wherein the reverse primer of a primer pair hybridizing specifically to the ILV3 gene of Aspergillus niger hybridises to one of the following target sequences from nucleotide sequence accession number NT_166533.1:
   i. positions 540084-540103
82. The at least one primer pair of any one of clauses 57 to 81 wherein the forward primer of a primer pair hybridizing specifically to the ILV3 gene of Aspergillus flavus hybridises to one of the following target sequences from nucleotide sequence accession number NW_002477240.1:
   i. positions c382604-382586
83. The at least one primer pair of any one of clauses 57 to 82 wherein the reverse primer of a primer pair hybridizing specifically to the ILV3 gene of Aspergillus flavus hybridises to one of the following target sequences from nucleotide sequence accession number NW_002477240.1:
   i. positions 382519-382541
   ii. positions 382520-382541
84. The at least one primer pair of any one of clauses 57 to 83 wherein the forward primer of a primer pair hybridizing specifically to the ILV3 gene of Cryptococcus neoformans hybridises to one of the following target sequences from nucleotide sequence accession number NC_006693.1:
   i. Positions 702696-702717
   ii. Positions 702499-702520
   iii. Positions 702736-702757
   iv. Positions 702384-702403
   v. Positions 701384-701406
85. The at least one primer pair of any one of clauses 57 to 84 wherein the reverse primer of a primer pair hybridizing specifically to the ILV3 gene of Cryptococcus neoformans hybridises to one of the following target sequences from nucleotide sequence accession number NC_006693.1:
   i. Positions c702796-702777
   ii. Positions c702602-702582
   iii. Positions c702850-702831
   iv. Positions c702521-702500
   v. Positions c701499-701479
86. The at least one primer pair of any one of clauses 57 to 85 wherein:
   a. the forward and reverse primer hybridizing specifically to the ILV3 gene of the following Candida species
      i. Candida albicans
      ii. Candida dubliniensis
      iii. Candida tropicalis
      iv. Candida parapsilosis
      v. Candida glabrata
      vi. Candida krusei
      vii. Candida guilliermondii
      viii. Candida auris
      comprises, consists essentially of or consists of the nucleotide sequence of SEQ ID NO: 1 and SEQ ID NO: 2 respectively;
   b. the forward and reverse primer hybridizing specifically to the ILV3 gene of the following Aspergillus species
      i. Aspergillus fumigatus
      ii. Aspergillus niger
      iii. Aspergillus flavus
      comprises, consists essentially of or consists of the nucleotide sequence of SEQ ID NO: 70 and 71 or SEQ ID NO: 73 and 74 respectively;
   c. the forward and reverse primer hybridizing specifically to the ILV3 gene of Candida albicans comprises, consists essentially of or consists of the nucleotide sequence of SEQ ID NO: 4 and 5 or SEQ ID NO: 6 and 7 respectively;
   d. the forward and reverse primer hybridizing specifically to the ILV3 gene of Candida dubliniensis comprises, consists essentially of or consists of the nucleotide sequence of SEQ ID NO: 8 and 9, SEQ ID NO: 10 and 11 or SEQ ID NO: 12 and 13 respectively;
   e. the forward and reverse primer hybridizing specifically to the ILV3 gene of Candida tropicalis comprises, consists essentially of or consists of the nucleotide sequence of SEQ ID NO: 14 and 15 or SEQ ID NO: 16 and 17 respectively;
   f. the forward and reverse primer hybridizing specifically to the ILV3 gene of Candida parapsilosis comprises, consists essentially of or consists of the nucleotide sequence of SEQ ID NO: 18 and 19 or SEQ ID NO: 20 and 21 respectively;
   g. the forward and reverse primer hybridizing specifically to the ILV3 gene of Candida glabrata comprises, consists essentially of or consists of the nucleotide sequence of SEQ ID NO: 22 and 23, SEQ ID NO: 24 and 25, SEQ ID NO: 26 and 27 or SEQ ID NO: 28 and 29 respectively;
   h. the forward and reverse primer hybridizing specifically to the ILV3 gene of Candida krusei comprises, consists essentially of or consists of the nucleotide sequence of SEQ ID NO: 30 and 31, SEQ ID NO: 32 and 33, SEQ ID NO: 34 and 35, SEQ ID NO: 36 and 37 or SEQ ID NO: 38 and 39 respectively;
   i. the forward and reverse primer hybridizing specifically to the ILV3 gene of Candida guilliermondii comprises, consists essentially of or consists of the nucleotide sequence of SEQ ID NO: 40 and 41, SEQ ID NO: 42 and 43, SEQ ID NO: 44 and 45 or SEQ ID NO: 46 and 47 respectively;
   j. the forward and reverse primer hybridizing specifically to the ILV3 gene of Candida auris comprises, consists essentially of or consists of the nucleotide sequence of SEQ ID NO: 48 and 49, SEQ ID NO: 50 and 51, SEQ ID NO: 52 and 53, SEQ ID NO: 54 and 55, SEQ ID NO: 56 and 57, SEQ ID NO: 58 and 59, SEQ ID NO: 60 and 61, SEQ ID NO: 62 and 63, SEQ ID NO: 64 and 65, SEQ ID NO: 66 and 67 or SEQ ID NO: 68 and 69 respectively;
   k. the forward and reverse primer hybridizing specifically to the ILV3 gene of Aspergillus fumigatus comprises, consists essentially of or consists of the nucleotide sequence of SEQ ID NO: 76 and 77, SEQ ID NO: 79 and 80, SEQ ID NO: 82 and 80 or SEQ ID NO: 83 and 84 respectively;
   l. the forward and reverse primer hybridizing specifically to the ILV3 gene of Aspergillus niger comprises, consists essentially of or consists of the nucleotide sequence of SEQ ID NO: 87 and 86;
   m. the forward and reverse primer hybridizing specifically to the ILV3 gene of Aspergillus flavus comprises, consists essentially of or consists of the nucleotide sequence of SEQ ID NO: 90 and 89 or SEQ ID NO: 90 and 92 respectively; and/or
   n. the forward and reverse primer hybridizing specifically to the ILV3 gene of Cryptococcus neoformans comprises, consists essentially of or consists of the nucleotide sequence of SEQ ID NO: 93 and 94, SEQ ID NO: 96 and 97, SEQ ID NO: 99 and 100, SEQ ID NO: 102 and 103 or SEQ ID NO: 105 and 106 respectively.
87. The at least one primer pair according to any one of clauses 57 to 86 comprising at least two primers pairs wherein at least one primer in each primer pair is differentially labelled compared to the other primer pairs.
88. At least one probe for detecting a yeast/fungus infection in a sample comprising:
   a. a probe that hybridizes specifically to the ILV3 gene of the following Candida species
      i. Candida albicans
      ii. Candida dubliniensis
      iii. Candida tropicalis
      iv. Candida parapsilosis
      v. Candida glabrata
      vi. Candida krusei
      vii. Candida guilliermondii
      viii. Candida auris
   b. a probe that hybridizes specifically to the ILV3 gene of the following Aspergillus species
      i. Aspergillus fumigatus
      ii. Aspergillus niger
      iii. Aspergillus flavus
   c. a probe that hybridizes specifically to the ILV3 gene of Candida albicans
   d. a probe that hybridizes specifically to the ILV3 gene of Candida dubliniensis
   e. a probe that hybridizes specifically to the ILV3 gene of Candida tropicalis
   f. a probe that hybridizes specifically to the ILV3 gene of Candida parapsilosis
   g. a probe that hybridizes specifically to the ILV3 gene of Candida glabrata
   h. a probe that hybridizes specifically to the ILV3 gene of Candida krusei
   i. a probe that hybridizes specifically to the ILV3 gene of Candida guilliermondii
   j. a probe that hybridizes specifically to the ILV3 gene of Candida auris
   k. a probe that hybridizes specifically to the ILV3 gene of Aspergillus fumigatus
   l. a probe that hybridizes specifically to the ILV3 gene of Aspergillus niger
   m. a probe that hybridizes specifically to the ILV3 gene of Aspergillus flavus; and/or
   n. a probe that hybridizes specifically to the ILV3 gene of Cryptococcus neoformans.
89. The at least one probe of clause 88 that hybridises to at least 3, 4, 5, 6, 7 and preferably all of the following target sequences:
   i. positions 1169779-1169804 of nucleotide sequence accession number NC_032093.1 (Candida albicans)
   ii. positions 393752-393777 of nucleotide sequence accession number NC_005968.1 (Candida glabrata)
   iii. positions 405950-405975 of nucleotide sequence accession number NW_003020040.1 (Candida tropicalis)
   iv. positions 1217990-1218015 of nucleotide sequence accession number NC_012864.1 (Candida dubliniensis)
   v. positions 909763-909788 of nucleotide sequence accession number NW_001809800.1 (Candida guilliermondii)
   vi. positions 23960-23985 of nucleotide sequence accession number HE605203.1 (Candida parapsilosis)
   vii. positions c61712-61687 of nucleotide sequence accession number JQFK01000016.1 (Candida krusei)
   viii. positions 32278-32303 of nucleotide sequence accession number NW_017263971.1 (Candida auris).
90. The at least one probe of any one of clauses 88 or 89 wherein:
   a. the probe hybridizing specifically to the ILV3 gene of Candida albicans hybridises to one of the following target sequences from nucleotide sequence accession number NC_032093.1:
   i. positions c1170174-1170151
   ii. positions 1171165-1171192
   b. the probe hybridizing specifically to the ILV3 gene of Candida dubliniensis hybridises to one of the following target sequences from nucleotide sequence accession number NC_012864.1:
   i. positions 1219148-1219171
   ii. positions c1218439-1218416
   iii. positions c1218505-1218480;
   c. the probe hybridizing specifically to the ILV3 gene of Candida tropicalis hybridises to one of the following target sequences from nucleotide sequence accession number NW_003020040.1:
   i. positions c406111-406082;
   d. the probe hybridizing specifically to the ILV3 gene of Candida parapsilosis hybridises to one of the following target sequences from nucleotide sequence accession number HE605203.1:
      i. positions c24907-24883
      ii. positions c24862-24839
   e. the probe hybridizing specifically to the ILV3 gene of Candida glabrata hybridises to one of the following target sequences from nucleotide sequence accession number NC_005968.1:
      i. positions c394151-394128
      ii. positions c393478-393453
      iii. positions c395030-395007
      iv. positions c394836-394807
   f. the probe hybridizing specifically to the ILV3 gene of Candida krusei hybridises to one of the following target sequences from nucleotide sequence accession number JQFK01000016.1:
      i. positions 61770-61793
      ii. positions c60990-60967
      iii. positions 60462-60485
      iv. positions 61815-61838
      v. positions c61970-61945
   g. the probe hybridizing specifically to the ILV3 gene of Candida guilliermondii hybridises to one of the following target sequences from nucleotide sequence accession number NW_001809800.1:
      i. positions c909577-909552
      ii. positions c911088-911065
      iii. positions 910878-910904; and/or
   h. the probe hybridizing specifically to the ILV3 gene of Candida auris hybridises to one of the following target sequences from nucleotide sequence accession number NW_017263971.1:
      i. positions c32744-32721
      ii. positions c32421-32398
      iii. positions c32629-32605
      iv. positions 33233-33257
      v. positions 32542-32563
      vi. positions 32350-32373
      vii. positions c33627-33606
      viii. positions c32218-32196
      ix. positions c32890-32864
      x. positions c33259-33236
      xi. positions c32061-32038
91. The at least one probe of any one of clauses 88 to 90 wherein at least 1 probe hybridises to at least 2, and preferably all 3, of the following target sequences:
   i. positions 3721790-3721814 of nucleotide sequence accession number NC_007195.1 (Aspergillus fumigatus)
   ii. positions c540811-540788 of nucleotide sequence accession number NT_166533.1 (Aspergillus niger)
   iii. positions c382405-382381 of nucleotide sequence accession number NW_002477240.1 (Aspergillus flavus)
   or wherein the common probe hybridises to at least 2, and preferably all 3, of the following target sequences:
   iv. positions 3721848-3721870 of nucleotide sequence accession number NC_007195.1 (Aspergillus fumigatus)
   v. positions c540753-540731 of nucleotide sequence accession number NT_166533.1 (Aspergillus niger)
   vi. positions c382347-382325 of nucleotide sequence accession number NW_002477240.1 (Aspergillus flavus).
92. The at least one probe of any one of clauses 88 to 91 wherein:
   a. the probe hybridizing specifically to the ILV3 gene of Aspergillus fumigatus hybridises to one of the following target sequences from nucleotide sequence accession number NC_007195.1:
      i. positions c3721651-3721628
      ii. positions 3721658-3721681
      iii. positions 3721848-3721870
   i. the probe hybridizing specifically to the ILV3 gene of Aspergillus niger hybridises to one of the following target sequences from nucleotide sequence accession number NT_166533.1:
      i. positions c540161-540138; and/or
   j. the probe hybridizing specifically to the ILV3 gene of Aspergillus flavus hybridises to one of the following target sequences from nucleotide sequence accession number NW_002477240.1:
      i. positions c382572-382549
93. The at least one probe of any one of clauses 88 to 92 wherein the probe hybridizing specifically to the ILV3 gene of Cryptococcus neoformans hybridises to one of the following target sequences from nucleotide sequence accession number NC_006693.1:
   i. Positions 702724-702747
   ii. Positions 702526-702549
   iii. Positions 702805-702829
   iv. Positions 702423-702446
   v. Positions 701435-701458
94. The at least one probe of any one of clauses 88 to 93 wherein:
   a. the probe that hybridizes specifically to the ILV3 gene of the following Candida species
      i. Candida albicans
      ii. Candida dubliniensis
      iii. Candida tropicalis
      iv. Candida parapsilosis
      v. Candida glabrata
      vi. Candida krusei
      vii. Candida guilliermondii
      viii. Candida auris
      comprises, consists essentially of or consists of the nucleotide sequence of SEQ ID NO: 3
   b. the probe that hybridizes specifically to the ILV3 gene of Candida albicans comprises, consists essentially of or consists of the nucleotide sequence of SEQ ID NO: 116 or 117
   c. the probe that hybridizes specifically to the ILV3 gene of Candida dubliniensis comprises, consists essentially of or consists of the nucleotide sequence of SEQ ID NO: 118, 119 or 120
   d. the probe that hybridizes specifically to the ILV3 gene of Candida tropicalis comprises, consists essentially of or consists of the nucleotide sequence of SEQ ID NO: 121
   e. the probe that hybridizes specifically to the ILV3 gene of Candida parapsilosis comprises, consists essentially of or consists of the nucleotide sequence of SEQ ID NO: 122 or 123
   f. the probe that hybridizes specifically to the ILV3 gene of Candida glabrata comprises, consists essentially of or consists of the nucleotide sequence of SEQ ID NO: 124, 125, 126 or 127
   g. the probe that hybridizes specifically to the ILV3 gene of Candida krusei comprises, consists essentially of or consists of the nucleotide sequence of SEQ ID NO: 128, 129, 130, 131 or 132
   h. the probe that hybridizes specifically to the ILV3 gene of Candida guilliermondii comprises, consists essentially of or consists of the nucleotide sequence of SEQ ID NO: 133, 134 or 135
   i. the probe that hybridizes specifically to the ILV3 gene of Candida auris comprises, consists essentially of or consists of the nucleotide sequence of SEQ ID NO: 136, 137, 138, 139, 140, 141, 142, 143, 144, 145 or 146
   j. The probe that hybridizes specifically to the ILV3 gene of the following Aspergillus species
      i. Aspergillus fumigatus
      ii. Aspergillus niger
      iii. Aspergillus flavus
      comprises, consists essentially of or consists of the nucleotide sequence of SEQ ID NO: 72 or 75
   k. the probe that hybridizes specifically to the ILV3 gene of Aspergillus fumigatus comprises, consists essentially of or consists of the nucleotide sequence of SEQ ID NO: 78, 81 or 85
   l. the probe that hybridizes specifically to the ILV3 gene of Aspergillus niger comprises, consists essentially of or consists of the nucleotide sequence of SEQ ID NO: 88
   m. the probe that hybridizes specifically to the ILV3 gene of Aspergillus flavus comprises, consists essentially of or consists of the nucleotide sequence of SEQ ID NO: 91; and/or
   n. the probe that hybridizes specifically to the ILV3 gene of Cryptococcus neoformans comprises, consists essentially of or consists of the nucleotide sequence of SEQ ID NO: 95, 98, 101, 104 or 107.
95. The at least one probe according to any one of clauses 88 to 94 comprising at least two probes wherein each probe is differentially labelled.
96. A kit for detecting a yeast/fungus infection in a sample comprising at least one primer pair according to any one of clauses 57 to 87 and/or at least one probe according to any one of clauses 88 to 95.
97. The kit of clause 96 comprising primer pairs in combination permitting detection of Candida, Aspergillus and Cryptococcus neoformans.
98. The kit of clause 97 further comprising probes permitting detection of Candida, Aspergillus and Cryptococcus neoformans.
99. A kit comprising the primers of SEQ ID NOs: 1 and 2, SEQ ID NOs: 70 and 71 and SEQ ID NOs: 93 and 94.
100. The kit of clause 99 further comprising the probes of SEQ ID NOs: 3, 72 and 95.
101. A kit for identifying the species responsible for a Candida infection in a sample, comprising the primer pairs of any one of clauses 57c to 57j or clauses 62 to 77.
102. The kit of clause 101 wherein the primers comprise, consist essentially of or consist of the nucleotide sequences of SEQ ID NO: 48 and 49, SEQ ID NO: 18 and 19, SEQ ID NO: 24 and 25, SEQ ID NO: 40 and 41, SEQ ID NO: 6 and 7, SEQ ID NO: 8 and 9, SEQ ID NO: 16 and 17 and SEQ ID NO: 38 and 39 respectively.
103. A kit for identifying the species responsible for an Aspergillus infection in a sample, comprising the primer pairs of any one of clauses 57k to 57m or clauses 78 to 83.
104. The kit of clause 103 wherein the primers comprise, consist essentially of or consist of the nucleotide sequences of SEQ ID NO: 80 and 82, SEQ ID NO: 86 and 87, SEQ ID NO: 90 and 92.
105. The kit according to any one of clauses 96 to 104 further comprising reagents for extracting DNA from a blood sample.
106. A method of detecting and identifying a microbial infection in a sample, comprising:
   a. performing a nucleic acid amplification reaction comprising the following components:
   i. a forward and reverse primer hybridizing specifically to the 16S rRNA gene of Gram positive bacteria; optionally together with a probe that hybridizes between the primer binding sites specifically to the 16S rRNA gene of Gram positive bacteria
   ii. a forward and reverse primer hybridizing specifically to the 16S rRNA gene of Gram negative bacteria; optionally together with a probe that hybridizes between the primer binding sites specifically to the 16S rRNA gene of Gram negative bacteria
   iii. a forward and reverse primer hybridizing specifically to the ILV3 gene of at least one fungal/yeast species; optionally together with a probe that hybridizes between the primer binding sites specifically to the ILV3 gene of at least one fungal/yeast species
   b. detecting and distinguishing the amplification products to determine whether the sample contains a Gram negative bacterial infection, a Gram positive bacterial infection and/or a fungal/yeast infection.
107. The method of clause 106 in which the amplification of the ILV3 gene is performed according to a method as defined in any one of clauses 1 to 56 and/or using at least one primer pair as defined in any one of clauses 57 to 87 and/or at least one probe according to any one of clauses 88 to 95 and/or using a kit as defined in any one of clauses 96 or 105.
108. A kit for discriminating a microbial infection in a sample, comprising components for performing a multiplex nucleic acid amplification reaction comprising:
   a. a forward and reverse primer hybridizing specifically to the 16S rRNA gene of Gram positive bacteria; optionally together with a probe that hybridizes between the primer binding sites specifically to the 16S rRNA gene of Gram positive bacteria
   b. a forward and reverse primer hybridizing specifically to the 16S rRNA gene of Gram negative bacteria; optionally together with a probe that hybridizes between the primer binding sites specifically to the 16S rRNA gene of Gram negative bacteria
   c. a forward and reverse primer hybridizing specifically to the ILV3 gene of at least one fungal/yeast species; optionally together with a probe that hybridizes between the primer binding sites specifically to the ILV3 gene of at least one fungal/yeast species;
   wherein components a, b and c each produce distinguishable amplification products thus enabling a determination of whether the sample contains a Gram negative bacterial infection, a Gram positive bacterial infection and/or a fungal/yeast infection.
109. The kit of clause 108 which comprises at least one primer pair as defined in any one of clauses 57 to 87 and/or at least one probe according to any one of clauses 88 to 95 and/or a kit as defined in any one of clauses 96 to 105.
110. A method of identifying the species responsible for a Candida infection in a sample, comprising:
   a. performing nucleic acid amplification reactions using at least three, 4, 5, 6, 7 or all of the following sets of components:
      i. a forward and reverse primer hybridizing specifically to the ILV3 gene of Candida albicans
      ii. a forward and reverse primer hybridizing specifically to the ILV3 gene of Candida dubliniensis
      iii. a forward and reverse primer hybridizing specifically to the ILV3 gene of Candida tropicalis
      iv. a forward and reverse primer hybridizing specifically to the ILV3 gene of Candida parapsilosis
      v. a forward and reverse primer hybridizing specifically to the ILV3 gene of Candida glabrata
      vi. a forward and reverse primer hybridizing specifically to the ILV3 gene of Candida krusei
      vii. a forward and reverse primer hybridizing specifically to the ILV3 gene of Candida guilliermondii
      viii. a forward and reverse primer hybridizing specifically to the ILV3 gene of Candida auris
   b. detecting and distinguishing the amplification products to identify the species responsible for the Candida infection.
111. The method of clause 110 wherein the primer pairs are used in a multiplex reaction.
112. The method of clause 110 or 111 wherein detecting and distinguishing the amplification products is according to a melt curve analysis.
113. The method of any one of clauses 110 to 112 wherein each primer pair is used in a separate reaction vessel.
114. The method of any one of clauses 110 to 113 wherein the forward primer hybridizing specifically to the ILV3 gene of Candida albicans hybridises to one of the following target sequences from nucleotide sequence accession number NC_032093.1:
   i. positions c1170234-1170217
   ii. positions c1171213-1171192
115. The method of any one of clauses 110 to 114 wherein the reverse primer hybridizing specifically to the ILV3 gene of Candida albicans hybridises to one of the following target sequences from nucleotide sequence accession number NC_032093.1:
   i. positions 1170119-1170140
   ii. positions 1171120-1171138
116. The method of any one of clauses 110 to 115 wherein the forward primer hybridizing specifically to the ILV3 gene of Candida dubliniensis hybridises to one of the following target sequences from nucleotide sequence accession number NC_012864.1:
   i. positions c1219181-1219161
   ii. positions c1218505-1218486
   iii. positions c1218553-1218534
117. The method of any one of clauses 110 to 116 wherein the reverse primer hybridizing specifically to the ILV3 gene of Candida dubliniensis hybridises to one of the following target sequences from nucleotide sequence accession number NC_012864.1:
   i. positions 1219103-1219124
   ii. positions 1218409-1218429
   iii. positions 1218419-1218440
118. The method of any one of clauses 110 to 117 wherein the forward primer hybridizing specifically to the ILV3 gene of Candida tropicalis hybridises to one of the following target sequences from nucleotide sequence accession number NW_003020040.1:
   i. positions c406146-406127
   ii. positions c406142-406120
119. The method of any one of clauses 110 to 118 wherein the reverse primer hybridizing specifically to the ILV3 gene of Candida tropicalis hybridises to one of the following target sequences from nucleotide sequence accession number NW_003020040.1:
   i. positions 406047-406068
   ii. positions 406048-406069
120. The method of any one of clauses 110 to 119 wherein the forward primer hybridizing specifically to the ILV3 gene of Candida parapsilosis hybridises to one of the following target sequences from nucleotide sequence accession number HE605203.1:
   i. positions c24951-24931
   ii. positions c24881-24863
121. The method of any one of clauses 110 to 120 wherein the reverse primer hybridizing specifically to the ILV3 gene of Candida parapsilosis hybridises to one of the following target sequences from nucleotide sequence accession number HE605203.1:
   i. positions 24843-24865
   ii. positions 24774-24795
122. The method of any one of clauses 110 to 121 wherein the forward primer hybridizing specifically to the ILV3 gene of Candida glabrata hybridises to one of the following target sequences from nucleotide sequence accession number NC_005968.1:
   i. positions c394191-394169
   ii. positions c393535-393516
   iii. positions c395076-395055
   iv. positions c394884-394863
123. The method of any one of clauses 110 to 122 wherein the reverse primer hybridizing specifically to the ILV3 gene of Candida glabrata hybridises to one of the following target sequences from nucleotide sequence accession number NC_005968.1:
   i. positions 394080-394101
   ii. positions 393445-393464
   iii. positions 394987-395006
   iv. positions 394783-394803
124. The method of any one of clauses 110 to 123 wherein the forward primer hybridizing specifically to the ILV3 gene of Candida krusei hybridises to one of the following target sequences from nucleotide sequence accession number JQFK01000016.1:
   i. positions 61723-61744
   ii. positions 60940-60961
   iii. positions 60420-60440
   iv. positions 61778-61797
   v. positions 61940-61961
125. The method of any one of clauses 110 to 124 wherein the reverse primer hybridizing specifically to the ILV3 gene of Candida krusei hybridises to one of the following target sequences from nucleotide sequence accession number JQFK01000016.1:
   i. positions c61817-61796
   ii. positions c61030-61011
   iii. positions c60535-60513
   iv. positions c61923-61902
   v. positions c62015-61996
126. The method of any one of clauses 110 to 125 wherein the forward primer hybridizing specifically to the ILV3 gene of Candida guilliermondii hybridises to one of the following target sequences from nucleotide sequence accession number NW_001809800.1:
   i. positions c909629-909608
   ii. positions c911117-911098
   iii. positions c911111-911090
   iv. positions c910941-910920
127. The method of any one of clauses 110 to 126 wherein the reverse primer hybridizing specifically to the ILV3 gene of Candida guilliermondii hybridises to one of the following target sequences from nucleotide sequence accession number NW_001809800.1:
   i. positions 909529-909550
   ii. positions 911002-911023
   iii. positions 910994-911013
   iv. positions 910799-910819
128. The method of any one of clauses 110 to 127 wherein the forward primer hybridizing specifically to the ILV3 gene of Candida auris hybridises to one of the following target sequences from nucleotide sequence accession number NW_017263971.1:
   i. positions c32790-32768
   ii. positions c32451-32430
   iii. positions c32654-32633
   iv. positions c33278-33259
   v. positions c32603-32580
   vi. positions c32399-32376
   vii. positions c33648-33628
   viii. positions c32240-32219
   ix. positions c32930-32909
   x. positions c33228-33206
   xi. positions c32106-32085
129. The method of any one of clauses 110 to 128 wherein the reverse primer hybridizing specifically to the ILV3 gene of Candida auris hybridises to one of the following target sequences from nucleotide sequence accession number NW_017263971.1:
   i. positions 32682-32703
   ii. positions 32368-32389
   iii. positions 32567-32588
   iv. positions 33202-33223
   v. positions 32512-32533
   vi. positions 32328-32349
   vii. positions 33566-33587
   viii. positions 32175-32195
   ix. positions 32826-32848
   x. positions 33130-33151
   xi. positions 32011-32033
130. The method of any one of clauses 110 to 129 wherein
   a. the forward and reverse primer hybridizing specifically to the ILV3 gene of Candida albicans comprises, consists essentially of or consists of the nucleotide sequence of SEQ ID NO: 4 and 5 or SEQ ID NO: 6 and 7 respectively;
   b. the forward and reverse primer hybridizing specifically to the ILV3 gene of Candida dubliniensis comprises, consists essentially of or consists of the nucleotide sequence of SEQ ID NO: 8 and 9, SEQ ID NO: 10 and 11 or SEQ ID NO: 12 and 13 respectively;
   c. the forward and reverse primer hybridizing specifically to the ILV3 gene of Candida tropicalis comprises, consists essentially of or consists of the nucleotide sequence of SEQ ID NO: 14 and 15 or SEQ ID NO: 16 and 17 respectively;
   d. the forward and reverse primer hybridizing specifically to the ILV3 gene of Candida parapsilosis comprises, consists essentially of or consists of the nucleotide sequence of SEQ ID NO: 18 and 19 or SEQ ID NO: 20 and 21 respectively;
   e. the forward and reverse primer hybridizing specifically to the ILV3 gene of Candida glabrata comprises, consists essentially of or consists of the nucleotide sequence of SEQ ID NO: 22 and 23, SEQ ID NO: 24 and 25, SEQ ID NO: 26 and 27 or SEQ ID NO: 28 and 29 respectively;
   f. the forward and reverse primer hybridizing specifically to the ILV3 gene of Candida krusei comprises, consists essentially of or consists of the nucleotide sequence of SEQ ID NO: 30 and 31, SEQ ID NO: 32 and 33, SEQ ID NO: 34 and 35, SEQ ID NO: 36 and 37 or SEQ ID NO: 38 and 39 respectively;
   g. the forward and reverse primer hybridizing specifically to the ILV3 gene of Candida guilliermondii comprises, consists essentially of or consists of the nucleotide sequence of SEQ ID NO: 40 and 41, SEQ ID NO: 42 and 43, SEQ ID NO: 44 and 45 or SEQ ID NO: 46 and 47 respectively;
   h. the forward and reverse primer hybridizing specifically to the ILV3 gene of Candida auris comprises, consists essentially of or consists of the nucleotide sequence of SEQ ID NO: 48 and 49, SEQ ID NO: 50 and 51, SEQ ID NO: 52 and 53, SEQ ID NO: 54 and 55, SEQ ID NO: 56 and 57, SEQ ID NO: 58 and 59, SEQ ID NO: 60 and 61, SEQ ID NO: 62 and 63, SEQ ID NO: 64 and 65, SEQ ID NO: 66 and 67 or SEQ ID NO: 68 and 69 respectively;
131. A method of identifying the species responsible for an Aspergillus infection in a sample, comprising:
   a. performing nucleic acid amplification reactions using at least two or all three of the following sets of components:
      i. a forward and reverse primer hybridizing specifically to the ILV3 gene of Aspergillus fumigatus
      ii. a forward and reverse primer hybridizing specifically to the ILV3 gene of Aspergillus niger
      iii. a forward and reverse primer hybridizing specifically to the ILV3 gene of Aspergillus flavus
   b. detecting and distinguishing the amplification products to identify the species responsible for the Aspergillus infection.
132. The method of clause 131 wherein detecting and distinguishing the amplification products is according to a melt curve analysis.
133. The method of clause 131 or 132 wherein the primer pairs are used in a multiplex reaction.
134. The method of any one of clauses 131 to 133 wherein each primer pair is used in a separate reaction vessel.
135. The method of any one of clauses 131 to 134 wherein the forward primer hybridizing specifically to the ILV3 gene of Aspergillus fumigatus hybridises to one of the following target sequences from nucleotide sequence accession number NC_007195.1:
   i. positions 3721531-3721552
   ii. positions 3721618-3721638
   iii. positions 3721616-3721638
   iv. positions 3721798-3721818
136. The method of any one of clauses 131 to 135 wherein the reverse primer hybridizing specifically to the ILV3 gene of Aspergillus fumigatus hybridises to one of the following target sequences from nucleotide sequence accession number NC_007195.1:
   i. positions c3721653-3721675
   ii. positions c3721706-3721681
   iii. positions c3721898-3721878
137. The method of any one of clauses 131 to 136 wherein the forward primer hybridizing specifically to the ILV3 gene of Aspergillus niger hybridises to one of the following target sequences from nucleotide sequence accession number NT_166533.1:
   i. positions c540219-540199
138. The method of any one of clauses 131 to 137 wherein the reverse primer hybridizing specifically to the ILV3 gene of Aspergillus niger hybridises to one of the following target sequences from nucleotide sequence accession number NT_166533.1:
   i. positions 540084-540103
139. The method of any one of clauses 131 to 138 wherein the forward primer hybridizing specifically to the ILV3 gene of Aspergillus flavus hybridises to one of the following target sequences from nucleotide sequence accession number NW_002477240.1:
   i. positions c382604-382586
140. The method of any one of clauses 131 to 139 wherein the reverse primer hybridizing specifically to the ILV3 gene of Aspergillus flavus hybridises to one of the following target sequences from nucleotide sequence accession number NW_002477240.1:
   i. positions 382519-382541
   ii. positions 382520-382541
141. The method of any one of clauses 131 to 140 wherein:
   a. the forward and reverse primer hybridizing specifically to the ILV3 gene of Aspergillus fumigatus comprises, consists essentially of or consists of the nucleotide sequence of SEQ ID NO: 76 and 77, SEQ ID NO: 79 and 80, SEQ ID NO: 82 and 80 or SEQ ID NO: 83 and 84 respectively;
   b. the forward and reverse primer hybridizing specifically to the ILV3 gene of Aspergillus niger comprises, consists essentially of or consists of the nucleotide sequence of SEQ ID NO: 87 and 86;
   c. the forward and reverse primer hybridizing specifically to the ILV3 gene of Aspergillus flavus comprises, consists essentially of or consists of the nucleotide sequence of SEQ ID NO: 90 and 89 or SEQ ID NO: 90 and 92 respectively.
142. The method of any one of clauses 110 to 141 which comprises use of a species specific probe for detecting and distinguishing the amplification products to thus identify the species responsible for the infection.
143. The method of clause 142 wherein each probe is differentially labelled.
144. The method of clause 142 or 143 wherein:
   a. the probe hybridizing specifically to the ILV3 gene of Candida albicans hybridises to one of the following target sequences from nucleotide sequence accession number NC_032093.1:
      i. positions c1170174-1170151
      ii. positions 1171165-1171192
   b. the probe hybridizing specifically to the ILV3 gene of Candida dubliniensis hybridises to one of the following target sequences from nucleotide sequence accession number NC_012864.1:
      i. positions 1219148-1219171
      ii. positions c1218439-1218416
      iii. positions c1218505-1218480;
   c. the probe hybridizing specifically to the ILV3 gene of Candida tropicalis hybridises to one of the following target sequences from nucleotide sequence accession number NW_003020040.1:
      i. positions c406111-406082;
   d. the probe hybridizing specifically to the ILV3 gene of Candida parapsilosis hybridises to one of the following target sequences from nucleotide sequence accession number HE605203.1:
      i. positions c24907-24883
      ii. positions c24862-24839
   e. the probe hybridizing specifically to the ILV3 gene of Candida glabrata hybridises to one of the following target sequences from nucleotide sequence accession number NC_005968.1:
      i. positions c394151-394128
      ii. positions c393478-393453
      iii. positions c395030-395007
      iv. positions c394836-394807
   f. the probe hybridizing specifically to the ILV3 gene of Candida krusei hybridises to one of the following target sequences from nucleotide sequence accession number JQFK01000016.1:
      i. positions 61770-61793
      ii. positions c60990-60967
      iii. positions 60462-60485
      iv. positions 61815-61838
      v. positions c61970-61945
   g. the probe hybridizing specifically to the ILV3 gene of Candida guilliermondii hybridises to one of the following target sequences from nucleotide sequence accession number NW_001809800.1:
      i. positions c909577-909552
      ii. positions c911088-911065
      iii. positions 910878-910904; and/or
   h. the probe hybridizing specifically to the ILV3 gene of Candida auris hybridises to one of the following target sequences from nucleotide sequence accession number NW_017263971.1:
      i. positions c32744-32721
      ii. positions c32421-32398
      iii. positions c32629-32605
      iv. positions 33233-33257
      v. positions 32542-32563
      vi. positions 32350-32373
      vii. positions c33627-33606
      viii. positions c32218-32196
      ix. positions c32890-32864
      x. positions c33259-33236
      xi. positions c32061-32038
145. The method of any one of clauses 142 to 144 wherein:
   a. the probe that hybridizes specifically to the ILV3 gene of Candida albicans comprises, consists essentially of or consists of the nucleotide sequence of SEQ ID NO: 116 or 117
   b. the probe that hybridizes specifically to the ILV3 gene of Candida dubliniensis comprises, consists essentially of or consists of the nucleotide sequence of SEQ ID NO: 118, 119 or 120
   c. the probe that hybridizes specifically to the ILV3 gene of Candida tropicalis comprises, consists essentially of or consists of the nucleotide sequence of SEQ ID NO: 121
   d. the probe that hybridizes specifically to the ILV3 gene of Candida parapsilosis comprises, consists essentially of or consists of the nucleotide sequence of SEQ ID NO: 122 or 123
   e. the probe that hybridizes specifically to the ILV3 gene of Candida glabrata comprises, consists essentially of or consists of the nucleotide sequence of SEQ ID NO: 124, 125, 126 or 127
   f. the probe that hybridizes specifically to the ILV3 gene of Candida krusei comprises, consists essentially of or consists of the nucleotide sequence of SEQ ID NO: 128, 129, 130, 131 or 132
   g. the probe that hybridizes specifically to the ILV3 gene of Candida guilliermondii comprises, consists essentially of or consists of the nucleotide sequence of SEQ ID NO: 133, 134 or 135; and/or
   h. the probe that hybridizes specifically to the ILV3 gene of Candida auris comprises, consists essentially of or consists of the nucleotide sequence of SEQ ID NO: 136, 137, 138, 139, 140, 141, 142, 143, 144, 145 or 146
146. The method of any one of clauses 142 to 145 wherein:
   a. the probe hybridizing specifically to the ILV3 gene of Aspergillus fumigatus hybridises to one of the following target sequences from nucleotide sequence accession number NC_007195.1:
      i. positions c3721651-3721628
      ii. positions 3721658-3721681
      iii. positions 3721848-3721870
   d. the probe hybridizing specifically to the ILV3 gene of Aspergillus niger hybridises to one of the following target sequences from nucleotide sequence accession number NT_166533.1:
      i. positions c540161-540138; and/or
   e. the probe hybridizing specifically to the ILV3 gene of Aspergillus flavus hybridises to one of the following target sequences from nucleotide sequence accession number NW_002477240.1:
      i. positions c382572-382549
147. The method of any one of clauses 142 to 146 wherein:
   a. the probe that hybridizes specifically to the ILV3 gene of Aspergillus fumigatus comprises, consists essentially of or consists of the nucleotide sequence of SEQ ID NO: 78, 81 or 85;
   b. the probe that hybridizes specifically to the ILV3 gene of Aspergillus niger comprises, consists essentially of or consists of the nucleotide sequence of SEQ ID NO: 88;
   c. the probe that hybridizes specifically to the ILV3 gene of Aspergillus flavus comprises, consists essentially of or consists of the nucleotide sequence of SEQ ID NO: 91.
148. A method of detecting and identifying a yeast/fungal infection in a sample comprising:
   a. Performing a method according to any one of clauses 1 to 56 in order to determine whether Candida, Aspergillus and/or Cryptococcus neoformans is present in the sample
   b. In the event that a species of Candida or Aspergillus is present in the sample performing a method according to any one of clauses 110 to 147 in order to determine which species is present
   to thereby detect and identify the yeast/fungal infection in the sample.
149. A method of selecting a subject for treatment with an antifungal agent such as a fungicide (or an antibiotic if bacteria are detected) comprising performing a method according to any one of clauses 1 to 56 and/or 106 to 148 and selecting the subject for treatment where an infection is detected, optionally also identified.
150. A method of predicting responsiveness of a subject to treatment with an antifungal agent such as a fungicide (or an antibiotic if bacteria are detected) comprising performing a method according to any one of clauses 1 to 56 and/or 106 to 149 and predicting responsiveness of the subject to treatment an infection is detected, optionally also identified.
151. A method of treating an infection comprising administering an antifungal agent such as a fungicide (or an antibiotic if bacteria are detected) to the subject suffering from the infection, wherein the subject has been selected for treatment by performing a method according to any one of clauses 1 to 56 and/or 106 to 150.
152. A method of treating an infection comprising administering an antifungal agent such as a fungicide (or an antibiotic if bacteria are detected) to the subject suffering from the infection, wherein the subject displays, in a sample, a detectable ILV3 gene.
153. An antifungal agent such as a fungicide (or an antibiotic if bacteria are detected) for use in a method of treating an infection, wherein the subject has been selected for treatment by performing a method according to any one of clauses 1 to 56 and/or 106 to 151.
154. An antifungal agent such as a fungicide for use in a method of treating an infection, wherein the subject displays, in a sample, a detectable ILV3 gene.

### DESCRIPTION OF THE FIGURES

FIG. 1 shows amplification curves in which the identification of 8 different Candida species was achieved by using a single primer-probe set containing degenerate bases (pan-Candida set).

FIG. 2 is an overlay of the melt curves from multiple Candida species using the best single primer-probe set for each species.

FIGS. 3A (set 1) and 3B (set 2) show amplification curves in which two pan-Aspergillus primer-probe sets yielded amplification for the three Aspergillus species tested.

FIGS. 4A and 4B show amplification curves from experiments to determine which primer-probe sets gave maximal performance, in terms of (a low) Ct detection value and (a high) level of fluorescence at the end of the amplification protocol.

### DETAILED DESCRIPTION (EXPERIMENTAL EXAMPLES)

The ILV3 gene represents a novel gene for the detection of Candida and other fungal organisms (including Aspergillus spp. and Cryptococcus neoformans). The ILV3 gene, which encodes for di-hydroxyacid dehydratase, an enzyme that catalyses the third step in the common pathway leading to biosynthesis of branched-chain amino acids, is a yeast/fungal-specific gene with 0% (zero) homology to any human DNA (Liu et al., 2006). Current fungal detection methods focus on ribosomal DNA (either 18S, internal transcribed spacers (5.8S) or 28S rRNA), which have large regions of homology to equivalent genes found in humans (ref: Khot et al., 2009; and Kan, 1992). This high level of homology makes the generation of specific primers and detection probes both challenging and time consuming as primer-probe sets need to be checked for their cross-reactivity to human DNA. Also, considering the products and methods of the invention will often use as the test sample lysates derived from human blood samples, any residual human DNA will be randomly sheared which may increase the likelihood of a human DNA sequence having complementarity, and thus cross-reactivity, to a ribosomal rRNA-based fungal primer-set. In contrast, with the use of an ILV3-based primer-probe set this screening process is not needed, and the risk of cross-reactivity to human DNA is eliminated. In parallel to targeting ILV3, it was also decided to employ hydrolysis (“TAQMAN”) probes for sample detection. TAQMAN probes allow for excellent sensitivity, specificity and qPCR performance to be achieved. However, in other embodiments, alternative probe types may be used. For example MOLECULAR BEACONS or SCORPIONS may be used to target ILV3 for the detection of yeast and fungi.

In addition to targeting ILV3 for the detection of Candida, this gene can also be targeted for, but not limited to, the detection of other fungal pathogens including several species of Aspergillus, as well as Cryptococcus neoformans.

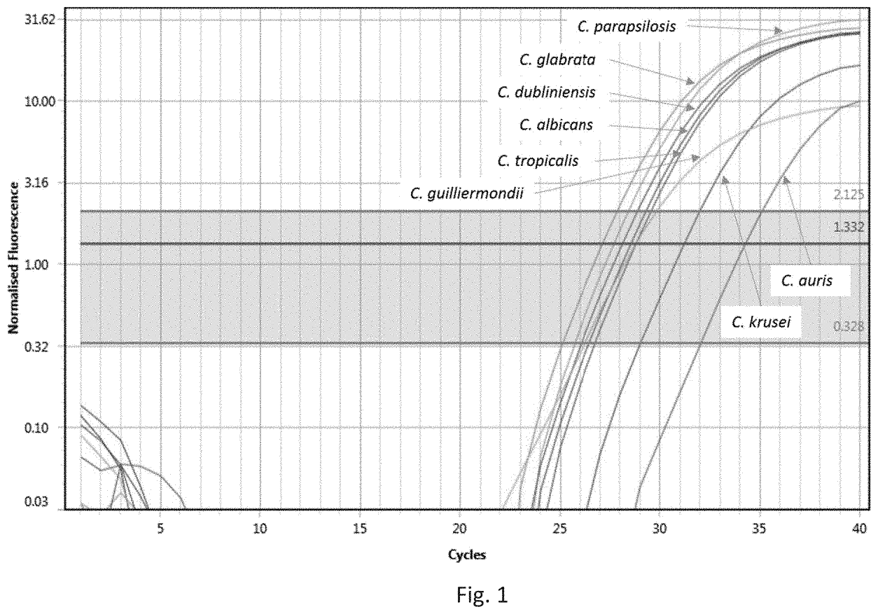

For the identification of Candida, bioinformatic analysis of the ILV3 gene (through sequence alignment of this gene against multiple Candida species) identified two regions that could be used for the detection of Candida species. Experiments demonstrated that the identification of multiple (8) species could be achieved, by using a single primer-probe set, which contained degenerate bases (a ‘pan-Candida’ primer-probe set; Primer-probe Set2—See Table 1 and FIG. 1.

TABLE 1

| Primer-probe | Ct (dR) | Organism | Primer-probe | Ct (dR) | Organism | Primer-probe | Ct (dR) |
|---|---|---|---|---|---|---|---|
| Set 1 - a/sen | No Ct | C. albicans (#7) | Set 1 - a/sen | No Ct | C. parapsilosis | Set 1 - a/sen | No Ct |
| Set 1 - a/sen | No Ct | C. albicans (#7) | Set 1 - a/sen | No Ct | C. parapsilosis | Set 1 - a/sen | No Ct |
| Set 1 - a/sen | No Ct | C. albicans (#7) | Set 1 - a/sen | No Ct | C. parapsilosis | Set 1 - a/sen | No Ct |
| Set 1 - sense | No Ct | C. albicans (#7) | Set 1 - sense | No Ct | C. parapsilosis | Set 1 - sense | No Ct |
| Set 1 - sense | No Ct | C. albicans (#7) | Set 1 - sense | No Ct | C. parapsilosis | Set 1 - sense | No Ct |
| Set 1 - sense | No Ct | C. albicans (#7) | Set 1 - sense | No Ct | C. parapsilosis | Set 1 - sense | No Ct |
| Set 2 - a/sen | 39.66 | C. albicans (#7) | Set 2 - a/sen | 36.4 | C. parapsilosis | Set 2 - a/sen | 38.06 |
| Set 2 - a/sen | 40.04 | C. albicans (#7) | Set 2 - a/sen | 37.49 | C. parapsilosis | Set 2 - a/sen | 39.05 |
| Set 2 - a/sen | 39.9 | C. albicans (#7) | Set 2 - a/sen | 37.49 | C. parapsilosis | Set 2 - a/sen | 38.74 |
| Set 2 - sense | 43.79 | C. albicans (#7) | Set 2 - sense | 41 | C. parapsilosis | Set 2 - sense | 41.99 |
| Set 2 - sense | 43.07 | C. albicans (#7) | Set 2 - sense | 38.79 | C. parapsilosis | Set 2 - sense | 41.26 |
| Set 2 - sense | 43.84 | C. albicans (#7) | Set 2 - sense | 38.68 | C. parapsilosis | Set 2 - sense | 41.49 |
| Set 1 - a/sen | No Ct | C. glabrata | Set 1 - a/sen | No Ct | NTC | Set 1 - sense | No Ct |
| Set 1 - a/sen | No Ct | C. glabrata | Set 1 - a/sen | No Ct | NTC | Set 1 - a/sen | No Ct |
| Set 1 - a/sen | No Ct | C. glabrata | Set 1 - a/sen | No Ct | NTC | Set 2 - sense | No Ct |
| Set 1 - sense | No Ct | C. glabrata | Set 1 - sense | No Ct | NTC | Set 2 - a/sen | No Ct |
| Set 1 - sense | No Ct | C. glabrata | Set 1 - sense | No Ct | | | |
| Set 1 - sense | No Ct | C. glabrata | Set 1 - sense | No Ct | | | |
| Set 2 - a/sen | 42.59 | C. glabrata | Set 2 - a/sen | 33.95 | | | |
| Set 2 - a/sen | 40.97 | C. glabrata | Set 2 - a/sen | 34.71 | | | |
| Set 2 - a/sen | 41.8 | C. glabrata | Set 2 - a/sen | 35.84 | | | |
| Set 2 - sense | 45 | C. glabrata | Set 2 - sense | 36.86 | | | |
| Set 2 - sense | No Ct | C. glabrata | Set 2 - sense | 37.8 | | | |
| Set 2 - sense | 44.25 | C. glabrata | Set 2 - sense | 40 | | | |

In order to further increase the performance of Candida detection (to enhance sensitivity and specificity) numerous variants of the original primer-probe set were designed and tested by substituting bases within the sequence of the primers and probe. The modifications to the reverse primer sequence and the probe sequence did not have any beneficial effect (data not shown). However, one of the re-designed forward primers enhanced the sensitivity of detection of a previously detectable Candida species (i.e. lower Ct (Cp) value relative to the original forward primer—an increase in the Delta Ct), as well as enhancing the performance specificity of detecting Candida guilliermondii, which previously had poor detection with the original primer-probe design—see Table 2. This new, updated set (SEQ ID NO: 1-3) has now been adopted (sequences shown in Table B).

TABLE 2
Rori + F2 + Pori

| Name | Cp | RFU | Delta Ct |
|---|---|---|---|
| CA | 28.25 | 5.387 | 0.5 |
| CT | 28.82 | 4.264 | 0.53 |
| CGI | 27.21 | 5.667 | 0.79 |
| CK | 30.59 | 3.323 | −0.07 |
| CP | 28.28 | 5.313 | −0.43 |
| CL | 35 | 1.001 | −0.89 |
| CGu | 27.31 | 1.465 | 4.31 |
| CDu | 28.8 | 5.321 | −0.58 |
| Rod + Fori + Pori | | | |
| Name | Cp | RFU | |
| CA | 28.75 | 4.212 | |
| CT | 29.35 | 3.495 | |
| CGI | 28 | 5.222 | |
| CK | 30.52 | 3.008 | |
| CP | 27.85 | 5.163 | |
| CL | 34.11 | 1.553 | |
| CGu | 31.62 | 0.713 | |
| CDu | 28.22 | 5.424 | |

A recent Public Health England report lists the individual species of Candida associated with candidaemia (“Surveillance of candidaemia in England, Wales and Northern Ireland, 2014”). From this report, it was decided to target the following Candida species whose genomes had been sequenced and published: C. albicans, C. glabrata, C. parapsilosis, C. tropicalis, C. krusei, C. dubliniensis, and C. guilliermondii. In addition to these species, Candida auris, the eighth species, was also added to the above list of organisms to be targeted for species-specific identification. The reason for this is because this species has been identified by Public Health England (“Candida auris identified in England”) as a significant emerging fungal pathogen with sporadic cases of C. auris having been identified throughout England and other countries Worldwide. Furthermore, C. auris has been shown to have a propensity for transmission between hospital patients, as well as showing resistance to three main classes of antifungal drugs, including azoles (e.g. fluconazole). Additional longitudinal epidemiological data has shown a recent shift in the incidence of bloodstream infections caused by non-albicans species of Candida (Wisplinghoff et al., 2014). These species, for example C. glabrata and C. krusei, have a decreased susceptibility to anti-fungal agents such as fluconazole relative to other Candida species (Trick et al., 2002). Therefore, there is a clinical need to be able to discriminate and differentiate between these eight most prevalent Candida species, especially C auris. To achieve this level of differentiation, the ILV3 gene was again interrogated bioinformatically. Multiple primer sets were designed for each species individually, with bioinformatic analysis of each primer set being performed to determine the specificity of these primer sets to the species of interest and ensuring no homology (cross-reactivity) was seen to other Candida species. The resulting primer sets were tested by melt curve analysis, using SYBR Green chemistry. Each primer set would generate an amplified region of DNA (‘amplicon’) of a precise melting temperature (Tm). Experiments using all the primer sets were conducted in order to determine the Tm profile of each primer set, for each Candida species—see Table 3.

TABLE 3

| Candida species | SEQ IDs | Actual Tm (° C.) | St. dev (° C.) |
|---|---|---|---|
| C. albicans | 4, 5 | 80.9 | 0.04 |
| | 6, 7 | 78.51 | 0.01 |
| C. dubliniensis | 8, 9 | 77.56 | 0 |
| | 10, 11 | 79.6 | 0.04 |
| | 12, 13 | 80.55 | 0.01 |
| C. tropicalis | 14, 15 | 76.93 | 0 |
| | 16, 17 | 76.33 | 0.01 |
| C. parapsilosis | 18, 19 | 81.03 | 0.1 |
| | 20, 21 | 77.38 | 0 |
| C. glabrata | 22, 23 | 79.27 | 0.12 |
| | 24, 25 | 79.79 | 0.04 |
| | 26, 27 | 80.39 | 0.08 |
| | 28, 29 | 76.6 | 0 |
| C. krusei | 30, 31 | 78.76 | 0.06 |
| | 32, 33 | 79.27 | 0.06 |
| | 34, 35 | 80.35 | 0 |
| | 36, 37 | 79.81 | 0 |
| | 38, 39 | 75.41 | 0.01 |
| C. guilliermondii | 40, 41 | 78.96 | 0.01 |
| | 42, 43 | 80.55 | 0 |
| | 44, 45 | 80.52 | 0.01 |
| | 46, 47 | 78.35 | 0 |
| C. auris | 48, 49 | 82.13 | 0.01 |
| | 50, 51 | 79.81 | 0.04 |
| | 52, 53 | 79.91 | 0.01 |
| | 54, 55 | 80.39 | 0.04 |
| | 56, 57 | 83.09 | 0.02 |
| | 58, 59 | 78.92 | 0.02 |
| | 60, 61 | 82.75 | 0 |
| | 62, 63 | 78.06 | 0.01 |
| | 64, 65 | 78.87 | 0.01 |
| | 66, 67 | 81.74 | 0 |
| | 68, 69 | 81.86 | 0.05 |

Once completed, the best single primer-probe set for each species was selected to optimise spread and separation between the Tm values of each species—see Table 4.

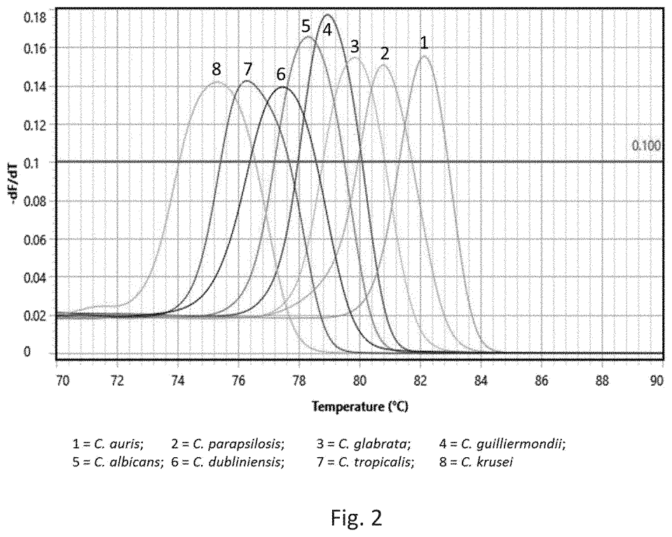

When these primer sets were again tested experimentally, and the melt curve trace of each species was overlaid, there was very good resolution between the eight traces, showing that this approach is a valid way of discriminating between individual Candida species—see FIG. 2. These primer sets provide delineation of more Candida species than the current test from T2 Biosystems.

TABLE 4

| Temperature | Candida | SEQ | Actual | Tm difference between |
|---|---|---|---|---|
| range (° C.) | Spp. | IDs | Tm (° C.) | next set (° C.) |
| 82.00 | CAu | 48, 49 | 82.13 | 1.34 |
| 81.75 | | | | |
| 81.50 | | | | |
| 81.25 | | | | |
| 81.00 | | | | |
| 80.75 | CP | 18, 19 | 80.79 | 0.88 |
| 80.50 | | | | |
| 80.25 | | | | |
| 80.00 | | | | |
| 79.75 | CGI | 24, 25 | 79.91 | 0.93 |
| 79.50 | | | | |
| 79.25 | | | | |
| 79.00 | CGu | 40, 41 | 78.98 | 0.65 |
| 78.75 | | | | |
| 78.50 | | | | |
| 78.25 | CA | 6, 7 | 78.33 | 0.865 |
| 78.00 | | | | |
| 77.75 | | | | |
| 77.50 | CDu | 8, 9 | 77.465 | 1.14 |
| 77.25 | | | | |
| 77.00 | | | | |
| 76.75 | | | | |
| 75.60 | | | | |
| 76.25 | CT | 16, 17 | 76.325 | 1.025 |
| 76.00 | | | | |
| 75.75 | | | | |
| 75.50 | | | | |
| 75.25 | CK | 38, 39 | 75.3 | |
| 75.00 | | | | |

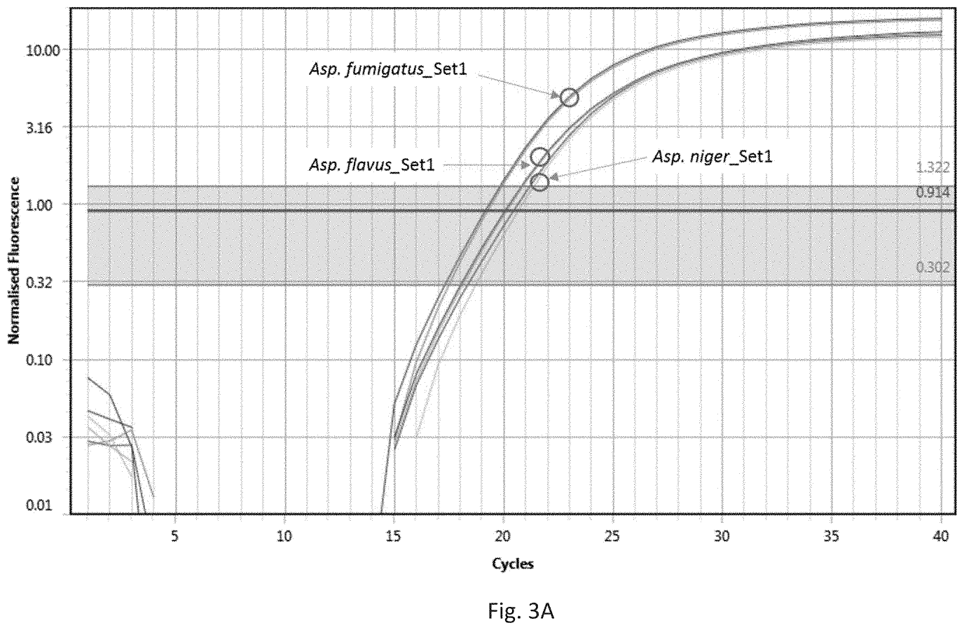

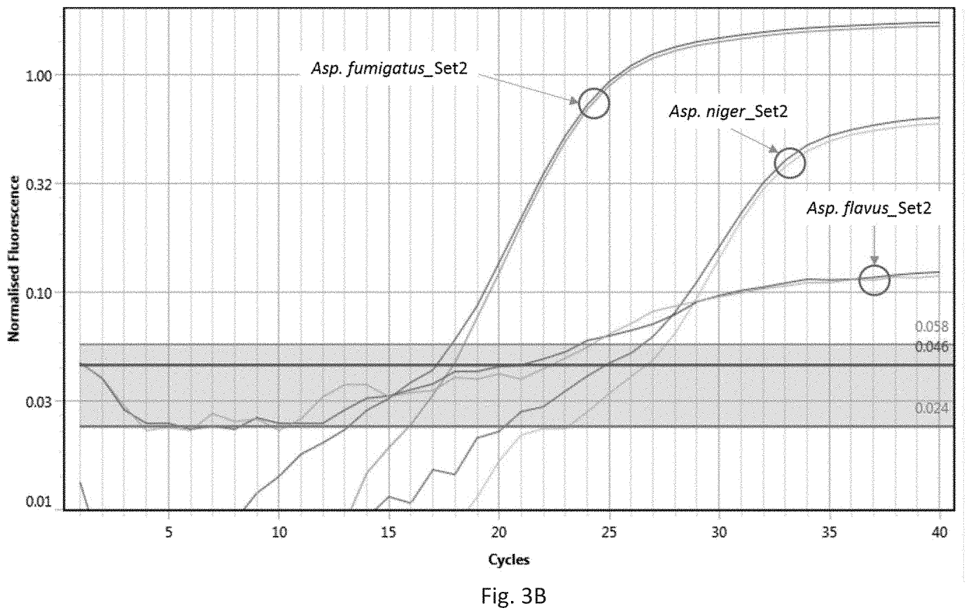

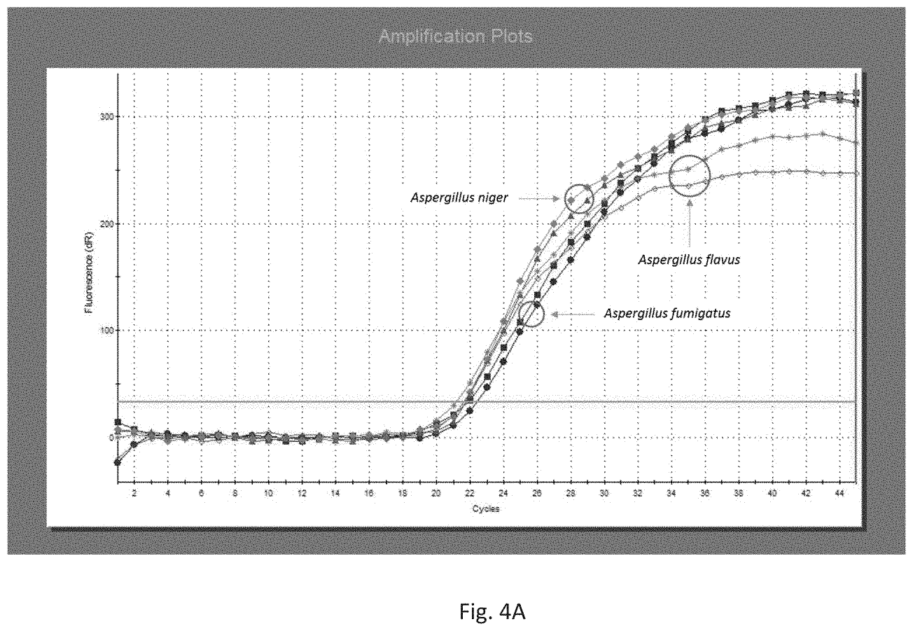

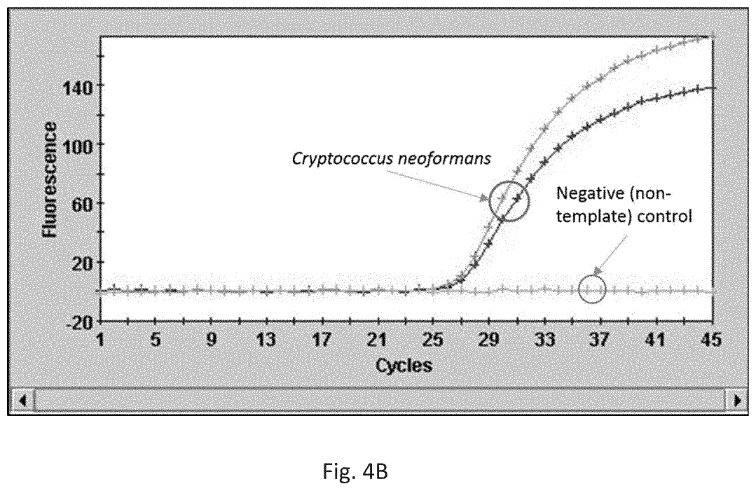

In addition to the detection of Candida species, ILV3 can also be used as a target for the detection of other fungal organisms such as Aspergillus species (particularly focused on Aspergillus fumigatus, Asp. niger, and Asp. flavus) and Cryptococcus neoformans. In an identical approach, as outlined previously for Candida, bioinformatic analysis and sequence alignment, where applicable, was used to identify suitable regions within the ILV3 gene for the design of a ‘pan-Aspergillus’ primer-probe set as well as several primer-probe sets for species-specific identification of the three Aspergillus species, as well as C. neoformans. Experiments demonstrated that two pan-Aspergillus primer-probe sets yielded amplification for the three Aspergillus species tested, but that the V1 design (SEQ ID Nos 70-72; FIG. 3A) was preferred due to better fluorescence across these species—see FIGS. 3A and 3B. For the generation of species-specific primer-probe sets, various design iterations for each species were tested bioinformatically to demonstrate species specificity (especially important for the detection of different Aspergillus species) as well as ensuring no homology to other fungal organisms or human genes. Primer-probe sets that passed this validation approach were then tested in vitro to determine which set gave maximal performance, in terms of (a low) Ct detection value and (a high) level of fluorescence at the end of the amplification protocol—see Tables 5-6. FIG. 4A shows amplification traces of the chosen species-specific primer-probe set for the three Aspergillus species (Asp. fumigatus: SEQ ID NO: 79-81; Asp. niger: SEQ ID NO: 86-88; and Asp. flavus: SEQ ID NO: 89-91. See Table B for sequences). FIG. 4B shows the amplification trace of the chosen primer-probe set for the detection of Cryptococcus neoformans (SEQ ID NO: 93-95).

TABLE 5

| SEQ IDs | Ct | Av. Ct | EndPt | Av. EndPt |
|---|---|---|---|---|
| 76, 77, 78 | 22.05 | 22.24 | 123.107 | 152.723 |
| | 22.43 | | 182.338 | |
| 79, 80, 81 | 22.96 | 22.65 | 345.802 | 331.714 |
| | 22.34 | | 317.626 | |
| 83, 84, 85 | 22.28 | 22.66 | 266.964 | 228.686 |
| | 23.03 | | 190.408 | |

TABLE 6

| SEQ IDs | Sample ID | Ct | EndPt |
|---|---|---|---|
| 93, 94, 95 | CN -1 | 27.2 | 138 |
| | CN - 1 | 26.9 | 174 |
| | NTC - 1 | 0 | 0 |
| 96, 97, 98 | CN -2 | 29.2 | 142 |
| | CN - 2 | 29.2 | 133 |
| | NTC - 2 | 0 | 0 |
| 99, 100, 101 | CN -3 | 27.5 | 81 |
| | CN - 3 | 27.1 | 92 |
| | NTC - 3 | 0 | 0 |
| 102, 103, 104 | CN -4 | 27.6 | 69 |
| | CN - 4 | 27.5 | 60 |
| | NTC 4 | 0 | 1 |
| 105, 106, 107 | CN -5 | 26.9 | 72 |
| | CN - 5 | 26.7 | 77 |
| | NTC -5 | 0 | −1 |

Alongside this, primer sets for melt curve analysis were tested to differentiate between the three Aspergillus species mentioned above. Again, the same bioinformatics approach used for the identification of Candida melt primers was used in order to find suitable Aspergillus melt primers. When these Aspergillus melt primers were tested in vitro a good resolution between the Tm value of each of the three Aspergillus species was achieved—see Tables 7-8.

TABLE 7

| Aspergillus species | SEQ IDs | Actual Tm (° C.) | St. dev (° C.) |
|---|---|---|---|
| Asp. fumigatus | 80, 82 | 82.95 | 0.01 |
| Asp. niger | 86, 87 | 79.54 | 0.01 |
| Asp. flavus | 90, 92 | 81.82 | 0.01 |

TABLE 8

| Temperature | Aspergillus | SEQ | Actual | Tm difference between |
|---|---|---|---|---|
| range (° C.) | species | IDs | Tm (° C.) | next set (° C.) |
| 83.00 | AFu | 80, 82 | 82.95 | 1.13 |
| 82.75 | | | | |
| 82.50 | | | | |
| 82.00 | | | | |
| 81.75 | AFI | 90, 92 | 81.82 | 2.28 |
| 81.50 | | | | |
| 81.25 | | | | |
| 81.00 | | | | |
| 80.75 | | | | |
| 80.50 | | | | |
| 80.25 | | | | |
| 80.00 | | | | |
| 79.75 | | | | |
| 79.50 | AN | 86, 87 | 79.54 | |
| 79.25 | | | | |
| 79.00 | | | | |

Each individual primer-probe set described herein, and shown in the accompanying Table B has been experimentally tested and validated, without any cross-reactivity of the various

ILV3 primer-probe sets to a non-target organism.

ILV3 represents a novel gene for the identification of yeast and fungal organisms, within the context of, but not limited to, confirming their presence within blood samples of patients suspected of having bloodstream infections.

### REFERENCES

Kan, V. L. (1993). Polymerase Chain Reaction for the Diagnosis of Candidemia. J. Infect. Dis. 168(3), 779-783.
Khot, P. D., Ko, D. L. & Fredricks, D. N. (2009). Sequencing and Analysis of Fungal rRNA Operons for Development of Broad-Range Fungal PCR Assays. Appl. Environ. Microbiol. 75(6), 1559-1565.
Liu, M., Healy, M. D., Dougherty, B. A., Esposito, K. M., Maurice, T. C., Mazzucco, C. E., Bruccoleri, R. E., Davison, D. B., Frosco, M., Barrett, J. F. & Wang, Y.-K. (2006). Conserved Fungal Genes as Potential Targets for Broad-Spectrum Antifungal Drug Discovery. Eukaryot. Cell. 5(4), 638-649.
Public Health England (2015). Surveillance of candidaemia in England, Wales and Northern Ireland, 2014. Vol. 9, No. 33; 18 Sep. 2015.
Public Health England (2016). www.gov.uk/government/publications/candida-auris-emergence-in-england. Published 1 Jul. 2016.
Trick, W. E., Fridkin, S. K., Edwards, J. R., Hajjeh, R. A., Gaynes, R. P., National Nosocomial Infections Surveillance System Hospitals (2002). Secular trend of hospital-acquired candidemia among intensive care unit patients in the United States during 1989-1999. Clin. Infect. Dis. 35(5), 627-630.
Wisplinghoff, H., Ebbers, J., Geurtz, L., Stefanik, D., Major, Y., Edmond, M. B., Wenzel, R. P., Seifert, H. (2014). Nosocomial bloodstream infections due to Candida spp. in the USA: species distribution, clinical features and antifungal susceptibilities. Int. J. Antimicrob. Agents. 43(1), 78-81.
Van Burik J A, Myerson D, Schreckhise R W, Bowden R A (1998). Panfungal PCR assay for detection of fungal infection in human blood specimens. J Clin Microbiol. 1998 May; 36(5):1169-75. 36(5):1169-75.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. A method comprising:
a. performing a nucleic acid amplification reaction and amplifying the ILV3 gene of fungi/yeast in a clinical sample obtained from a human subject, and
b. detecting the amplification product and determining whether the sample contains a fungal/yeast infection.

2. The method of claim 1 comprising:
a. performing a nucleic acid amplification reaction and amplifying the ILV3 gene of fungi/yeast in a clinical sample obtained from a human subject, the reaction comprising the following components:
i. a forward and reverse primer hybridizing specifically to the ILV3 gene of Candida species, optionally together with a probe that hybridizes between the primer binding sites specifically to the ILV3 gene of Candida species; and
ii. a forward and reverse primer hybridizing specifically to the ILV3 gene of Aspergillus species, optionally together with a probe that hybridizes between the primer binding sites specifically to the ILV3 gene of Aspergillus species; and/or
iii. a forward and reverse primer hybridizing specifically to the ILV3 gene of Cryptococcus neoformans, optionally together with a probe that hybridizes between the primer binding sites specifically to the ILV3 gene of Cryptococcus neoformans, and
b. detecting the amplification products and determining whether the sample contains a fungal/yeast infection.

3. The method of claim 1 wherein the nucleic acid amplification reaction amplifies the ILV3 gene of at least 3 of the following species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
ix. Aspergillus fumigatus
x. Aspergillus niger
xi. Aspergillus flavus
xii. Cryptococcus neoformans.

4. The method of claim 2 wherein step a comprises:
i. use of a forward and reverse primer hybridizing specifically to the ILV3 gene of Candida species, Aspergillus species or Cryptococcus neoformans; and/or
ii. use of a probe that hybridizes specifically to the ILV3 gene of Candida species, Aspergillus species or Cryptococcus neoformans.

5. The method of claim 2 wherein a common forward and reverse primer and/or common probe hybridises to the ILV3 gene of at least 3 of the following Candida species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris;
and/or wherein a common forward and reverse primer and/or probe hybridises to the ILV3 gene of at least 2 of the following Aspergillus species:
i. Aspergillus fumigatus
ii. Aspergillus niger
iii. Aspergillus flavus

6. The method of claim 2 wherein a separate forward and reverse primer and/or probe hybridises to the ILV3 gene of each of at least 3 of the following Candida species:
i. Candida albicans
ii. Candida dubliniensis
iii. Candida tropicalis
iv. Candida parapsilosis
v. Candida glabrata
vi. Candida krusei
vii. Candida guilliermondii
viii. Candida auris;
and/or wherein a separate forward and reverse primer and/or probe hybridises to the ILV3 gene of each of at least 2 of the following Aspergillus species:
i. Aspergillus fumigatus
ii. Aspergillus niger
iii. Aspergillus flavus.

7. The method of claim 1 wherein step b comprises distinguishing amplification products in order to identify the genus and/or species responsible for the infection.

8. The method of claim 1, wherein step b comprises detecting and distinguishing the amplification products to identify the fungal/yeast infection.

9. The method of claim 1, comprising:
a. performing a nucleic acid amplification reaction and amplifying the ILV3 gene of fungi/yeast in a clinical sample obtained from a human subject, the reaction comprising the following components:
i. a forward and reverse primer hybridizing specifically to the ILV3 gene of Candida species, optionally together with a probe that hybridizes between the primer binding sites specifically to the ILV3 gene of Candida species; and
ii. a forward and reverse primer hybridizing specifically to the ILV3 gene of Aspergillus species, optionally together with a probe that hybridizes between the primer binding sites specifically to the ILV3 gene of Aspergillus species; and/or
iii. a forward and reverse primer hybridizing specifically to the ILV3 gene of Cryptococcus neoformans, optionally together with a probe that hybridizes between the primer binding sites specifically to the ILV3 gene of Cryptococcus neoformans, and
b. detecting and distinguishing the amplification products to identify the fungal/yeast infection.

10. The method of claim 9 wherein distinguishing comprises:
i. a melting curve analysis
ii. use of differently labelled primers and/or probes; or
iii. determining the size of the amplification products.

11. The method of claim 10 wherein at least one primer and/or probe is differentially labelled according to genus to permit identification of the genus of fungus/yeast in the sample; optionally wherein at least one primer and/or probe is differentially labelled according to species of Candida and/or Aspergillus to permit identification of the species of Candida and/or Aspergillus in the sample.

12. The method of claim 1 wherein the sample comprises a blood sample.

13. A collection of primer pairs comprising:
A) a. a forward and reverse primer hybridizing specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
b. a forward and reverse primer hybridizing specifically to the ILV3 gene of the following Aspergillus species
i. Aspergillus fumigatus
ii. Aspergillus niger
iii. Aspergillus flavus; and
optionally a forward and reverse primer hybridizing specifically to the ILV3 gene of Cryptococcus neoformans; or
B) a. a forward and reverse primer hybridizing specifically to the ILV3 gene of Candida albicans
b. a forward and reverse primer hybridizing specifically to the ILV3 gene of Candida dubliniensis
c. a forward and reverse primer hybridizing specifically to the ILV3 gene of Candida tropicalis
d. a forward and reverse primer hybridizing specifically to the ILV3 gene of Candida parapsilosis
e. a forward and reverse primer hybridizing specifically to the ILV3 gene of Candida glabrata
f. a forward and reverse primer hybridizing specifically to the ILV3 gene of Candida krusei
g. a forward and reverse primer hybridizing specifically to the ILV3 gene of Candida guilliermondii
h. a forward and reverse primer hybridizing specifically to the ILV3 gene of Candida auris
i. a forward and reverse primer hybridizing specifically to the ILV3 gene of Aspergillus fumigatus;
j. a forward and reverse primer hybridizing specifically to the ILV3 gene of Aspergillus niger;
k. a forward and reverse primer hybridizing specifically to the ILV3 gene of Aspergillus flavus; and optionally
l. a forward and reverse primer hybridizing specifically to the ILV3 gene of Cryptococcus neoformans;
optionally wherein at least one primer in each primer pair is differentially labelled compared to the other primer pairs.

14.-43. (canceled)

44. A collection of probes comprising:
A) a. a probe that hybridizes specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
b. a probe that hybridizes specifically to the ILV3 gene of the following Aspergillus species
i. Aspergillus fumigatus
ii. Aspergillus niger
iii. Aspergillus flavus; and
optionally a probe that hybridizes specifically to the ILV3 gene of Cryptococcus neoformans; or
B) a. a probe that hybridizes specifically to the ILV3 gene of Candida albicans
b. a probe that hybridizes specifically to the ILV3 gene of Candida dubliniensis
c. a probe that hybridizes specifically to the ILV3 gene of Candida tropicalis
d. a probe that hybridizes specifically to the ILV3 gene of Candida parapsilosis
e. a probe that hybridizes specifically to the ILV3 gene of Candida glabrata
f. a probe that hybridizes specifically to the ILV3 gene of Candida krusei
g. a probe that hybridizes specifically to the ILV3 gene of Candida guilliermondii
h. a probe that hybridizes specifically to the ILV3 gene of Candida auris
i. a probe that hybridizes specifically to the ILV3 gene of Aspergillus fumigatus
j. a probe that hybridizes specifically to the ILV3 gene of Aspergillus niger
k. a probe that hybridizes specifically to the ILV3 gene of Aspergillus flavus; and optionally
l. a probe that hybridizes specifically to the ILV3 gene of Cryptococcus neoformans;
optionally wherein the collection of probes comprises at least two probes wherein each probe is differentially labelled.

45.-52. (canceled)

53. A kit comprising,
(1) A collection of primer pairs:
A) a. a forward and reverse primer hybridizing specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
c. a forward and reverse primer hybridizing specifically to the ILV3 gene of the following Aspergillus species
i. Aspergillus fumigatus
ii. Aspergillus niger
iii. Aspergillus flavus; and
optionally a forward and reverse primer hybridizing specifically to the ILV3 gene of Cryptococcus neoformans; or
B) a. a forward and reverse primer hybridizing specifically to the ILV3 gene of Candida albicans
b. a forward and reverse primer hybridizing specifically to the ILV3 gene of Candida dubliniensis
c. a forward and reverse primer hybridizing specifically to the ILV3 gene of Candida tropicalis
d. a forward and reverse primer hybridizing specifically to the ILV3 gene of Candida parapsilosis
e. a forward and reverse primer hybridizing specifically to the ILV3 gene of Candida glabrata
f. a forward and reverse primer hybridizing specifically to the ILV3 gene of Candida krusei
g. a forward and reverse primer hybridizing specifically to the ILV3 gene of Candida guilliermondii
h. a forward and reverse primer hybridizing specifically to the ILV3 gene of Candida auris
i. a forward and reverse primer hybridizing specifically to the ILV3 gene of Aspergillus fumigatus;
j. a forward and reverse primer hybridizing specifically to the ILV3 gene of Aspergillus niger;
k. a forward and reverse primer hybridizing specifically to the ILV3 gene of Aspergillus flavus; and optionally
l. a forward and reverse primer hybridizing specifically to the ILV3 gene of Cryptococcus neoformans;
optionally wherein at least one primer in each primer pair is differentially labelled compared to the other primer pairs; and
(2) A collection of probes comprising:
A) a. a probe that hybridizes specifically to the ILV3 gene of the following Candida species
i. Candida albicans
ii. Candida dubliniensis
iii. Candida tropicalis
iv. Candida parapsilosis
v. Candida glabrata
vi. Candida krusei
vii. Candida guilliermondii
viii. Candida auris
b. a probe that hybridizes specifically to the ILV3 gene of the following Aspergillus species
i. Aspergillus fumigatus
ii. Aspergillus niger
iii. Aspergillus flavus; and
optionally a probe that hybridizes specifically to the ILV3 gene of Cryptococcus neoformans; or
B) a. a probe that hybridizes specifically to the ILV3 gene of Candida albicans
b. a probe that hybridizes specifically to the ILV3 gene of Candida dubliniensis
c. a probe that hybridizes specifically to the ILV3 gene of Candida tropicalis
d. a probe that hybridizes specifically to the ILV3 gene of Candida parapsilosis
e. a probe that hybridizes specifically to the ILV3 gene of Candida glabrata
f. a probe that hybridizes specifically to the ILV3 gene of Candida krusei
g. a probe that hybridizes specifically to the ILV3 gene of Candida guilliermondii
h. a probe that hybridizes specifically to the ILV3 gene of Candida auris
i. a probe that hybridizes specifically to the ILV3 gene of Aspergillus fumigatus
j. a probe that hybridizes specifically to the ILV3 gene of Aspergillus niger
k. a probe that hybridizes specifically to the ILV3 gene of Aspergillus flavus; and optionally
l. a probe that hybridizes specifically to the ILV3 gene of Cryptococcus neoformans;
optionally wherein the collection of probes comprises at least two probes wherein each probe is differentially labelled.

54. A method comprising:
a. performing a nucleic acid amplification reaction and amplifying the 16S rRNA gene of Gram positive bacteria in a clinical sample obtained from a human subject, the reaction comprising the following components:
i. a forward and reverse primer hybridizing specifically to the 16S rRNA gene of Gram positive bacteria; optionally together with a probe that hybridizes between the primer binding sites specifically to the 16S rRNA gene of Gram positive bacteria
ii. a forward and reverse primer hybridizing specifically to the 16S rRNA gene of Gram negative bacteria; optionally together with a probe that hybridizes between the primer binding sites specifically to the 16S rRNA gene of Gram negative bacteria
iii. a forward and reverse primer hybridizing specifically to the ILV3 gene of at least one fungal/yeast species; optionally together with a probe that hybridizes between the primer binding sites specifically to the ILV3 gene of at least one fungal/yeast species
b. detecting and distinguishing the amplification products to determine and determining whether the sample contains a Gram negative bacterial infection, a Gram positive bacterial infection and/or a fungal/yeast infection.

55. A kit comprising:
a. a forward and reverse primer hybridizing specifically to the 16S rRNA gene of Gram positive bacteria; optionally together with a probe that hybridizes between the primer binding sites specifically to the 16S rRNA gene of Gram positive bacteria
b. a forward and reverse primer hybridizing specifically to the 16S rRNA gene of Gram negative bacteria; optionally together with a probe that hybridizes between the primer binding sites specifically to the 16S rRNA gene of Gram negative bacteria
c. a forward and reverse primer hybridizing specifically to the ILV3 gene of at least one fungal/yeast species; optionally together with a probe that hybridizes between the primer binding sites specifically to the ILV3 gene of at least one fungal/yeast species;
wherein components a, b and c each produce distinguishable amplification products thus enabling a determination of whether the sample contains a Gram negative bacterial infection, a Gram positive bacterial infection and/or a fungal/yeast infection.

56. (canceled)

57. The method of claim 1, comprising:
a. performing nucleic acid amplification reactions on the clinical sample obtained from the human subject using at least three, 4, 5, 6, 7 or all of the following sets of components:
i. a forward and reverse primer hybridizing specifically to the ILV3 gene of Candida albicans
ii. a forward and reverse primer hybridizing specifically to the ILV3 gene of Candida dubliniensis
iii. a forward and reverse primer hybridizing specifically to the ILV3 gene of Candida tropicalis
iv. a forward and reverse primer hybridizing specifically to the ILV3 gene of Candida parapsilosis
v. a forward and reverse primer hybridizing specifically to the ILV3 gene of Candida glabrata
vi. a forward and reverse primer hybridizing specifically to the ILV3 gene of Candida krusei
vii. a forward and reverse primer hybridizing specifically to the ILV3 gene of Candida guilliermondii
viii. a forward and reverse primer hybridizing specifically to the ILV3 gene of Candida auris
b. detecting and distinguishing the amplification products and identifying the species responsible for a Candida infection; optionally wherein detecting and distinguishing the amplification products is according to a melt curve analysis; optionally wherein each primer pair is used in a separate reaction vessel.

58. The method of claim 1, comprising:
a. performing nucleic acid amplification reactions on the clinical sample obtained from the human subject using at least two or all three of the following sets of components:
i. a forward and reverse primer hybridizing specifically to the ILV3 gene of Aspergillus fumigatus
ii. a forward and reverse primer hybridizing specifically to the ILV3 gene of Aspergillus niger
iii. a forward and reverse primer hybridizing specifically to the ILV3 gene of Aspergillus flavus
b. detecting and distinguishing the amplification products and identifying the species responsible for an Aspergillus infection; optionally wherein detecting and distinguishing the amplification products is according to a melt curve analysis; optionally wherein each primer pair is used in a separate reaction vessel.

59.-64. (canceled)
